(19) United States
(12) Patent Application Publication
(10) Pub. No.: US 2016/0158321 A1
(43) Pub. Date: Jun. 9, 2016

(54) TREATMENT OF PEDIATRIC GROWTH HORMONE DEFICIENCY WITH HUMAN GROWTH HORMONE ANALOGUES

(71) Applicant: AMUNIX OPERTING INC., Mountain View, CA (US)

(72) Inventors: Jeffrey L. CLELAND, San Carlos, CA (US); George M. BRIGHT, San Mateo, CA (US); Eric HUMPHRISS, Menlo Park, CA (US)

(21) Appl. No.: 14/771,445

(22) PCT Filed: Mar. 10, 2014
(86) PCT No.: PCT/US14/22850

§ 371 Date: Aug. 28, 2015

Related U.S. Application Data
(60) Provisional application No. 61/776,618, filed on Mar. 11, 2013.
(60) Provisional application No. 61/810,786, filed on Apr. 11, 2013.
(60) Provisional application No. 61/835,002, filed on Jun. 14, 2013.
(60) Provisional application No. 61/880,701, filed on Sep. 20, 2013.
(60) Provisional application No. 61/911,731, filed on Dec. 4, 2013.
(60) Provisional application No. 61/931,987, filed on Jan. 27, 2014.
(60) Provisional application No. 61/948,457, filed on Mar. 5, 2014.

(51) Int. Cl. A61K 38/27; A61K 38/17

(52) U.S. Cl. CPC A61K 38/27; A61K 38/17

(57) ABSTRACT

The present invention concerns a pediatric growth hormone deficiency (PGHD) therapy for pediatric subjects. The therapy comprises administering to the pediatric patient with PGHD a human growth hormone-XTEN (hGH-XTEN) fusion protein in therapeutically effective doses every week, every two weeks, semimonthly, every three weeks, or monthly. This therapy is not inferior compared to the height velocity achieved with daily injections of hGH not linked to XTEN over the same period.

### BACKGROUND OF THE INVENTION

Human growth hormone (hGH) is naturally secreted from the human anterior pituitary as intermittent pulses lasting from minutes to hours typically occurring during sleep. The rate and extent of hGH secretion decreases with aging and is maximal in puberty in normal healthy well nourished children. hGH binds to the hGH receptor initiating signaling processes involving the STAT (signal transducer and activator of transcription), the MAPK (mitogen-activated protein kinase) and the PI3K (phosphoinositide-3 kinase) pathways. Insulin-like growth factor-I (IGF-I) gene expression is activated from hGH receptor signaling resulting in secretion of IGF-I into the circulation. IGF-I forms a complex with insulin-like growth factor binding protein-3 (IGFBP-3) and the acid labile subunit (ALS). Both IGFBP-3 and ALS expression are also regulated by hGH receptor activation.

In children with growth hormone deficiency (GHD) resulting from lack of expression or secretion of hGH and not caused by a defect in the hGH receptor, replacement therapy with daily injections of rhGH is often prescribed to facilitate near normal growth and development. New bone is formed at the epiphyses in response to hGH and IGF-I resulting in linear growth until the growth plates fuse after puberty. Daily rhGH administration does not mimic the normal endogenous pulses of hGH in non-GHD children, but does result in significant increases in growth with a typical first year growth rate on treatment of 11 cm/yr. Clinical studies of continuous infusion of rhGH with a pump demonstrated comparable growth velocity and IGF-I levels to those achieved with daily rhGH injections (Jorgensen et al. J. Clin Endocrinol Metab. 70(6), 1616-23 (1990); Laursen, T. et al. J Clin Endocrinol Metab. 80(8), 2410-8 (1995); Tauber, M. et al. J Clin Endocrinol Metab. 76(5), 1135-9 (1993)). Therefore, continuous, as well as pulsatile, administration of rhGH is efficacious.

The safety of daily rhGH therapy has been studied in both GHD children and adults. In some overweight or obese patients, a trend toward increasing fasting and post-prandial insulin levels has been observed. Although generally well tolerated, daily rhGH therapy may cause mild to moderate headache, arthralgia, nausea, vomiting and injection reactions.

Others have reported on various sustained release GH preparations (Cook D M, et al. 2002. J Clin Endocrinol Metab 87(10):4508-4514; Biller B M, et al. 2011. J Clin Endocrinol Metab 96(6):1718-1726; Peter F. et al., 2012. J Clin Endocrinol Metab 97(2):400-407; Fares F. et al, 2010. Endocrinology 151(9):4410-4417; Sondergaard E, et al. 2011. J Clin Endocrinol Metab 96(3):681-688; de Schepper J et al. 2011. European Journal of Endocrinology 165(3):401-409; Bidlingmaier M, et al. 2006. J Clin Endocrinol Metab 91(8):2926-2930). However, there remains a need for alternative GH therapeutics, dosages, and treatment regimens.

VRS-317 is an investigational long-acting rhGH in development for long-term replacement therapy for adults with GHD and children with pediatric GHD. VRS-317 was designed to achieve up to once-monthly dosing with the anticipation that a reduced frequency of administration (as few as 12 versus up to 365 injections per year) would increase treatment adherence and thereby improve overall treatment outcomes. VRS-317 is an rhGH fusion protein that was designed to minimize receptor mediated clearance through a reduction in receptor binding achieved without mutations to rhGH by genetically fusing extended recombinant polypeptide (XTEN) amino acid sequences to the N- and C-termini of the native hGH sequence (Cleland et al. 2012, Journal of Pharmaceutical Sciences. 101(8):2744-2754, Epub 2012 Jun. 7).

### SUMMARY OF THE INVENTION

The present invention concerns an improved therapeutic regimen for pediatric growth hormone deficiency (“PGHD”) therapy in children. In particular, the invention concerns methods for bolus dose administration of compositions of fusion proteins comprising human growth hormone fused to one or more extended recombinant polypeptides (XTEN) (the fusion protein hereinafter referred to as “hGH-XTEN”). Accordingly, in one aspect, the present invention concerns a method of treating pediatric patients having human PGHD with an hGH-XTEN fusion protein.

In one aspect, the present invention provides a method of treating human pediatric growth hormone deficiency (PGHD) in a pediatric patient by administering to the patient with PGHD a dose of human growth hormone-XTEN (hGH-XTEN) fusion protein. In another embodiment, the hGH-XTEN fusion protein comprises an amino acid sequence having at least about 90% sequence identity to SEQ ID NO:1. In one other embodiment, the dose is a bolus dose. In one embodiment, the bolus dose of hGH-XTEN is a therapeutically effective bodyweight adjusted bolus dose. In another embodiment, the bolus dose of hGH-XTEN is between about 0.80 mg/kg and about 6.3 mg/kg. In another embodiment, the bolus dose of hGH-XTEN is between about 0.80 mg/kg and about 7.0 mg/kg.

In other embodiments, the bolus dose of hGH-XTEN is administered every week, every two weeks, semimonthly (i.e., occurring twice a month), every three weeks, or monthly. In another embodiment, the administration of the bolus dose of hGH-XTEN is monthly. In a preferred embodiment, the administration of the bolus dose of hGH-XTEN is weekly. In a preferred embodiment, the administration of the bolus dose of hGH-XTEN is semimonthly. In another preferred embodiment, the administration of the bolus dose of hGH-XTEN is in every three weeks. In additional embodiments, the bolus dose of hGH-XTEN is administered subcutaneously.

In an additional embodiment, the human pediatric patient has a serum IGF-I standard deviation score (SDS) between about −2.0 and about 2.0 following administration of a bolus dose of hGH-XTEN. In another embodiment, the human pediatric patient has a serum IGF-I standard deviation score (SDS) between about −2.0 and about 2.0 following a first, or a second, or a third, or a fourth bolus dose administration of a bolus dose of hGH-XTEN. In other embodiments, the pediatric patient exhibits said serum IGF-I SDS following administration of the bolus dose, wherein the IGF-I SDS is selected from the group consisting of greater than about −2.0, greater than about −1.5, greater than about −1.0, greater than about −0.5, greater than about 0, greater than about 0.5, greater than about 1.0, and greater than about 1.5. In other embodiments, the pediatric patient exhibits said serum IGF-I SDS following administration of the bolus dose, wherein the IGF-I SDS is selected from the group consisting of greater than about −1.5 to about 2.0, greater than about −1.0 to about 2.0, greater than about −0.5 to about 2.0, greater than about 0 to about 2.0, greater than about 0.5 to about 2.0, greater than about 1.0 to about 2.0, and greater than about 1.5 to about 2.0. In other embodiments, the pediatric patient exhibits said serum IGF-I SDS following administration of the bolus dose, wherein the IGF-I SDS is selected from the group consisting of greater than about −1.0 to about 2.0, greater than about 0 to about 2.0, and greater than about 1.0 to about 2.0. In another embodiment, the pediatric patient exhibits said serum IGF-I SDS following administration of the bolus dose, wherein the administration is weekly, every two weeks, every three weeks, or monthly. In another embodiment, the administration is weekly, every two weeks, semimonthly, every three weeks, or monthly. In an additional embodiment, the administration is semimonthly, or monthly. In other embodiments, the bolus dose is effective to maintain the pediatric patient's serum IGF-I SDS between about −2.0 and about 2.0 for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or at least about a month following administration. In other embodiments, the bolus dose is effective to maintain the pediatric patient's serum IGF-I SDS between about −2.0 and about 2.0 for at least about 14 days, at least about 21 days, or at least about 30 days following administration. In another embodiment, the bolus dose is effective to maintain the pediatric patient's serum IGF-I SDS between about −2.0 and about 2.0 for at least about 14 days, or at least about 30 days following administration. In another embodiment, the human pediatric patient has a serum IGF-I standard deviation score (SDS) between about −2.0 and about 2.0 following said days after a first, or a second, or a third, or a fourth bolus dose administration of hGH-XTEN.

In other embodiments, the bolus dose is effective to maintain the pediatric patient's serum IGF-I SDS baseline serum IGF-I standard deviation score (SDS) of at least 1.0 for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or at least about one month following administration. In another embodiment, the bolus dose is effective to maintain the pediatric patient's serum IGF-I SDS baseline serum IGF-I standard deviation score (SDS) of at least 1.0 for at least about 14 days, at least about 21 days, or at least about 30 days following administration. In an additional embodiment, the bolus dose is effective to maintain the pediatric patient's serum IGF-I SDS baseline serum IGF-I standard deviation score (SDS) of at least 1.0 for at least about 14 days, or at least about 30 days following administration. In other embodiments, the bolus dose is effective to maintain the pediatric patient's serum IGF-I SDS baseline serum IGF-I standard deviation score (SDS) of at least 1.0 following said days after a first, or a second, or a third, or a fourth bolus dose administration of hGH-XTEN.

In another embodiment, the pediatric patient exhibits said serum IGF-I standard deviation score (SDS) following administration of at least a second, or a third, or a fourth bolus dose.

In other embodiments, the invention provides a method of treating human pediatric growth hormone deficiency (PGHD) in a pediatric patient by administering a hGH-XTEN fusion protein to the patient wherein the hGH-XTEN is effective to achieve a height velocity equivalent to at least about 6 cm/yr, or at least about 7 cm/yr, or at least about 8 cm/yr, or at least about 9 cm/yr, or at least about 10 cm/yr, or at least about 11 cm/yr, or at least 12 cm/yr in a pediatric patient. In another embodiment, the bolus dose of hGH-XTEN is effective to achieve a height velocity equivalent between about 7 cm/yr to about 12 cm/yr. In other embodiment, the bolus dose of hGH-XTEN is effective to achieve a height velocity equivalent between about 8 cm/yr to 11 cm/yr in a pediatric patient. In the foregoing embodiments of the paragraph, the height velocity is achieved after at least 3 months, after at least 6 months, after at least 9 months, or after at least 12 months of dosing in the pediatric patient. In other embodiments, the height velocity achieved is a first year height velocity.

In yet other embodiments, the invention provides a method of treating human pediatric growth hormone deficiency (PGHD) in a pediatric patient by administering a hGH-XTEN fusion protein to the patient wherein the method is not inferior to achieve a height velocity in a pediatric patient compared with that achieved using daily injections of hGH not linked to XTEN over the same period. In one embodiment, the hGH-XTEN fusion protein administered is comparable, on a molar basis, to an equivalent amount of an hGH not linked to XTEN and administered to a pediatric patient. In one embodiment, the equivalent amount is selected from a an hGH dose of at least about 25, at least about 30, at least about 33, at least about 35, at least about 37, or at least about or at least about 40 μg hGH/kg/day.

In yet other embodiments, the invention provides a method of treating human pediatric growth hormone deficiency (PGHD) in a pediatric patient by administering a hGH-XTEN fusion protein to the patient wherein the method is effective to maintain the pediatric patient's height velocity within at least about 10%, at least about 20%, or at least about 30% of that compared to the height velocity achieved in pediatric patients administered daily injections of hGH not linked to XTEN of an equivalent amount, on a molar basis, over a comparable dose period. In one embodiment, the equivalent amount is selected from a an hGH dose of at least about 25, at least about 30, at least about 33, at least about 35, at least about 37, or at least about or at least about 40 μg hGH/kg/day.

In one embodiment, the bolus dose of hGH-XTEN is selected from the group consisting of about 0.8 mg/kg, about 1.0 mg/kg, about 1.2 mg/kg, about 1.4 mg/kg, about 1.6 mg/kg, about 1.8 mg/kg, about 2.0 mg/kg, about 2.2 mg/kg, about 2.4 mg/kg, about 2.6 mg/kg, about 2.7 mg/kg, about 2.8 mg/kg, about 3 mg/kg, about 3.2 mg/kg, about 3.4 mg/kg, about 3.6 mg/kg, about 3.8 mg/kg, about 4.0 mg/kg, about 4.2 mg/kg, about 4.4 mg/kg, about 4.6 mg/kg, about 4.8 mg/kg, about 5.0 mg/kg, about 5.2 mg/kg, about 5.4 mg/kg, about 5.6 mg/kg, about 5.8 mg/kg, about 6.0 mg/kg, and about 6.3 mg/kg. In another embodiment, the bolus dose is about 0.8 mg/kg to about 2.0 mg/kg. In another embodiment, the bolus dose is about 2.0 mg/kg to about 4.0 mg/kg. In another embodiment, the bolus dose is about 4.0 mg/kg to about 6.0 mg/kg. In another embodiment, the bolus dose is about 6.0 mg/kg to about 7.0 mg/kg. In another embodiment, the bolus dose is about 0.8 mg/kg to about 1.5 mg/kg. In another embodiment, the bolus dose is about 1.8 mg/kg to about 3.2 mg/kg. In another embodiment, the bolus dose is about 3.5 mg/kg to about 6.3 mg/kg.

In another embodiment, the hGH-XTEN fusion protein comprises the amino acid sequence of SEQ ID NO:1. In another embodiment, the hGH-XTEN fusion protein has at least about 91%, or at least about 92%, or at least about 93%, or at least about 94%, or at least about 95%, or at least about 96%, or at least about 97%, or at least about 98%, or at least about 99% sequence identity to SEQ ID NO:1.

In another aspect, the present invention provides a method of treating human pediatric growth hormone deficiency (PGHD) in a human pediatric patient by administering to the patient with PGHD a dose of human growth hormone-XTEN (hGH-XTEN) fusion protein that is effective to maintain the patient's serum IGF-I standard deviation score (SDS) at a certain level. In one embodiment, the method comprises administering an hGH-XTEN fusion protein with an amino acid sequence having at least about 90% sequence identity to SEQ ID NO:1. In another embodiment, the dose is a therapeutically effective bodyweight adjusted bolus dose. In one other embodiment, the bolus dose is effective to maintain the patient's serum IGF-I standard deviation score (SDS) between about −2.0 and about 2.0. In an additional embodiment, the bolus dose is effective to maintain the IGF-I SDS between about −2.0 and about 2.0 for at least 7 days after administration of the bolus dose. In other embodiments, the bolus dose of hGH-XTEN is between about 0.8 mg/kg and about 6.3 mg/kg. In one embodiment, the bolus dose of hGH-XTEN is effective to maintain the patient's serum IGF-I SDS between about −2.0 and about 2.0 for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or at least about a month following administration. In another embodiment, the bolus dose is effective to maintain the pediatric patient's serum IGF-I SDS between about −2.0 and about 2.0 for at least about 14 days, at least about 21 days, or at least about 30 days following administration. In another embodiment, the bolus dose is effective to maintain the pediatric patient's serum IGF-I SDS between about −2.0 and about 2.0 for at least about 14 days, or at least about 30 days following administration.

In one additional aspect, the present invention provides a pediatric bolus dose of an hGH-XTEN fusion protein. In one embodiment, the hGH-XTEN fusion protein comprises an amino acid sequence having at least about 90% sequence identity to SEQ ID NO:1. In another embodiment, the bolus dose is a therapeutically effective bodyweight adjusted bolus dose. In one other embodiment, the bolus dose of hGH-XTEN comprises between about 0.8 mg/kg and about 6.3 mg/kg of hGH-XTEN fusion protein. In other embodiments, the bolus dose is for use in treating human pediatric growth hormone deficiency (PGHD) in a pediatric patient in need. In another embodiment, the hGH-XTEN fusion protein comprises the amino acid sequence of SEQ ID NO:1. In one embodiment, the bolus dose of hGH-XTEN is formulated for subcutaneous administration.

In another aspect, the present invention provides an hGH-XTEN fusion protein (i) for use in a method of treating human pediatric growth hormone deficiency (PGHD) in a human pediatric patient; or (ii) for use in the manufacture of a medicament for the treatment of PGHD in a pediatric patient. In one embodiment, the hGH-XTEN fusion protein comprises an amino acid sequence having at least about 90% sequence identity to SEQ ID NO:1. In an additional embodiment, the method comprises administering a bolus dose of the hGH-XTEN fusion protein. In another embodiment, the medicament comprises a bolus dose of the hGH-XTEN fusion protein. In one other embodiment, the bolus dose is a therapeutically effective bodyweight adjusted bolus dose of the hGH-XTEN fusion protein. In one embodiment, the bolus dose is between about 0.8 mg/kg and about 6.3 mg/kg. In another embodiment, the bolus dose is administered every week, every two weeks, semimonthly, every three weeks, or monthly. In another embodiment, the bolus dose is administered every semimonthly, or monthly. In one additional embodiment, the hGH-XTEN fusion protein comprises the amino acid sequence of SEQ ID NO:1. In another embodiment, the bolus dose is administered subcutaneously. In another embodiment, the medicament is formulated for subcutaneous administration. In other embodiments, the human pediatric patient has a serum IGF-I standard deviation score (SDS) between about −2.0 and about 2.0 following administration of the bolus dose of hGH-XTEN. In one additional embodiment, the pediatric patient exhibits said serum IGF-I SDS following administration of the bolus dose, wherein the IGF-I SDS is selected from the group consisting of greater than about −2.0, greater than about −1.5, greater than about −1.0, greater than about −0.5, greater than about 0, greater than about 0.5, greater than about 1.0, and greater than about 1.5. In one embodiment, the bolus dose is administered weekly, every two weeks, every three weeks, semimonthly or monthly. In another embodiment, the bolus dose is administered semimonthly, or monthly. In another embodiment, the IGF-I SDS is selected from the group consisting of greater than about −1.5, greater than about −1.0, greater than about −0.5, greater than about 0, greater than about 0.5, greater than about 1.0, and greater than about 1.5. In another embodiment, the IGF-I SDS is selected from the group consisting of greater than about −1.0, greater than about 0, and greater than about 1.0.

In another embodiment, hGH-XTEN fusion protein administration is weekly, every two weeks, semimonthly, every three weeks, or monthly. In another embodiment, hGH-XTEN fusion protein administration is semimonthly, or monthly.

In one other aspect, the present invention provides a kit for the treatment of pediatric growth hormone deficiency (PGHD). In one embodiment, the kit comprises a container which holds a pharmaceutical composition comprising a human growth hormone-XTEN (hGH-XTEN) fusion protein. In another embodiment, the hGH-XTEN fusion protein comprises an amino acid sequence having at least about 90% sequence identity to SEQ ID NO:1. In one other embodiment, the kit further comprises a package insert associated with said container. In one additional embodiment, the package insert indicates that said composition is for the treatment of pediatric growth hormone deficiency (PGHD) in a pediatric patient by administration of an initial dose of the hGH-XTEN fusion protein. In another embodiment, the package insert further indicates administration of a plurality of subsequent doses of the hGH-XTEN fusion protein. In one other embodiment, the initial dose is between about 0.8 mg/kg and about 6.3 mg/kg. In an additional embodiment, the plurality of subsequent doses of the hGH-XTEN fusion protein is between about 0.8 mg/kg and about 6.3 mg/kg. In one embodiment, the doses of the hGH-XTEN fusion protein are administered every week, every two weeks, semimonthly, every three weeks, or monthly. In another embodiment, the doses of the hGH-XTEN fusion protein are administered, semimonthly, or monthly.

In another aspect, the present invention provides a human growth hormone-XTEN (hGH-XTEN) fusion protein for use in a pharmaceutical regimen for treatment of a treatment of pediatric growth hormone deficiency (PGHD) in a pediatric patient. In one embodiment, the hGH-XTEN fusion protein comprises an amino acid sequence having at least about 90% sequence identity to SEQ ID NO:1. In another embodiment, the pharmaceutical regimen comprises administering a bolus dose of the hGH-XTEN fusion protein to treat the pediatric patient. In one other embodiment, the bolus dose is a therapeutically effective bodyweight adjusted bolus dose of the hGH-XTEN fusion protein. In one embodiment, the bolus dose is between about 0.8 mg/kg and about 6.3 mg/kg. In one other embodiment, the pharmaceutical regimen further comprises the step of determining the amount of hGH-XTEN fusion protein needed to achieve an IGF-I standard deviation score (SDS) between about −2.0 and about 2.0 in the pediatric patient. In one embodiment, the pharmaceutical regimen for treating the pediatric patient comprises administering the hGH-XTEN fusion protein in an initial bolus dose between about 0.8 mg/kg and about 6.3 mg/kg and a plurality of subsequent bolus doses of the hGH-XTEN fusion protein between about 0.8 mg/kg and about 6.3 mg/kg. In another embodiment, the bolus doses are administered every week, every two weeks, semimonthly, every three weeks, or monthly. In another embodiment, the bolus doses are administered semimonthly, or monthly.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 provides the amino acid sequence for an hGH-XTEN fusion protein (hGH sequence is underlined and bold) (SEQ ID NO:1).

FIG. 2 summarizes the design for the Phase 1b/2a study of a human growth hormone-XTEN (hGH-XTEN) fusion protein in pediatric patients.

FIG. 3 shows the hGH-XTEN fusion protein plasma concentration (ng/mL) mean values.

FIG. 4 shows the hGH-XTEN fusion protein Cmax (ng/mL) and hGH-XTEN fusion protein AUC (hr-ng/mL).

FIG. 5 demonstrates a sustained change (from baseline) in IGF-I (mean values).

FIG. 6 demonstrates that IGF-I responses are linearly related to the dose of hGH-XTEN fusion protein.

FIG. 7 summarizes the design for the Phase 1b/2a study of a human growth hormone-XTEN (hGH-XTEN) fusion protein in pediatric patients. The hGH-XTEN fusion protein doses equivalent in recombinant hGH (rhGH) mass to 5-37 μg/kd/d taken for 30 days.

FIG. 8 provides a table showing the Clinical Characteristics of Completed Dosing Groups; Numerical values are means (SD).

FIG. 9 provides a table showing related adverse events considered as possibly, probably or definitely related to study drug in dose level groups 1-6. All related AE are mild (CTCAE Grade 1) and transient. No SAE, No unexpected AE, No patient withdrawals, No lipoatrophy, No nodules.

FIG. 10 shows the hGH-XTEN fusion protein plasma concentration (ng/mL) mean values (preliminary PK from Phase 1b).

FIG. 11 shows the hGH-XTEN fusion protein Cmax (ng/mL) and hGH-XTEN fusion protein AUC (hr-ng/mL) (dose proportionality).

FIG. 12A-B show IGF-I SDS responses to single doses of the fusion protein.

FIG. 13A-B show an increase from Baseline in Monthly Average IGF-I SDS (Single Dose). An increase in average IGF-I SDS increases with increasing dose (p<0.00001). A desired monthly IGF-I profile achieved.

FIG. 14 shows mean annualized height velocities for age-matched historical controls and VRS-317 treated patients.

### DESCRIPTION OF THE INVENTION

Before the embodiments of the invention are described, it is to be understood that such embodiments are provided by way of example only, and that various alternatives to the embodiments of the invention described herein may be employed in practicing the invention. Numerous variations, changes, and substitutions will now occur to those skilled in the art without departing from the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### DEFINITIONS

As used herein, the following terms have the meanings ascribed to them unless specified otherwise.

As used in the specification and claims, the singular forms “a”, “an” and “the” include plural references unless the context clearly dictates otherwise. For example, the term “a cell” includes a plurality of cells, including mixtures thereof.

The terms “polypeptide”, “peptide”, and “protein” are used interchangeably herein to refer to polymers of amino acids of any length. The polymer may be linear or branched, it may comprise modified amino acids, and it may be interrupted by non amino acids. The terms also encompass an amino acid polymer that has been modified, for example, by disulfide bond formation, glycosylation, lipidation, acetylation, phosphorylation, or any other manipulation, such as conjugation with a labeling component.

As used herein the term “amino acid” refers to either natural and/or unnatural or synthetic amino acids, including but not limited to glycine and both the D or L optical isomers, and amino acid analogs and peptidomimetics. Standard single or three letter codes are used to designate amino acids.

The term “natural L-amino acid” means the L optical isomer forms of glycine (G), proline (P), alanine (A), valine (V), leucine (L), isoleucine (I), methionine (M), cysteine (C), phenylalanine (F), tyrosine (Y), tryptophan (W), histidine (H), lysine (K), arginine (R), glutamine (Q), asparagine (N), glutamic acid (E), aspartic acid (D), serine (S), and threonine (T).

The term “non-naturally occurring,” as applied to sequences and as used herein, means polypeptide or polynucleotide sequences that do not have a counterpart to, are not complementary to, or do not have a high degree of homology with a wild-type or naturally-occurring sequence found in a mammal. For example, a non-naturally occurring polypeptide or fragment may share no more than 99%, 98%, 95%, 90%, 80%, 70%, 60%, 50% or even less amino acid sequence identity as compared to a natural sequence when suitably aligned.

The terms “hydrophilic” and “hydrophobic” refer to the degree of affinity that a substance has with water. A hydrophilic substance has a strong affinity for water, tending to dissolve in, mix with, or be wetted by water, while a hydrophobic substance substantially lacks affinity for water, tending to repel and not absorb water and tending not to dissolve in or mix with or be wetted by water. Amino acids can be characterized based on their hydrophobicity. A number of scales have been developed. An example is a scale developed by Levitt, M, et al., J Mol Biol (1976) 104:59, which is listed in Hopp, T P, et al., Proc Natl Acad Sci USA (1981) 78:3824. Examples of “hydrophilic amino acids” are arginine, lysine, threonine, alanine, asparagine, and glutamine. Of particular interest are the hydrophilic amino acids aspartate, glutamate, and serine, and glycine. Examples of “hydrophobic amino acids” are tryptophan, tyrosine, phenylalanine, methionine, leucine, isoleucine, and valine.

A “fragment” is a truncated form of a native biologically active protein that retains at least a portion of the therapeutic and/or biological activity. A “variant” is a protein with sequence homology to the native biologically active protein that retains at least a portion of the therapeutic and/or biological activity of the biologically active protein. For example, a variant protein may share at least 70%, 75%, 80%, 85%, 90%, 95%, 96%, 97%, 98% or 99% amino acid sequence identity with the reference biologically active protein. As used herein, the term “biologically active protein moiety” includes proteins modified deliberately, as for example, by site directed mutagenesis, insertions, or accidentally through mutations.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this invention.

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is generally greater than that of its naturally occurring counterpart. In general, a polypeptide made by recombinant means and expressed in a host cell is considered to be “isolated.”

An “isolated” polynucleotide or polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal or extra-chromosomal location different from that of natural cells.

A “chimeric” protein contains at least one fusion polypeptide comprising regions in a different position in the sequence than that which occurs in nature. The regions may normally exist in separate proteins and are brought together in the fusion polypeptide; or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship.

“Conjugated”, “linked,” “fused,” and “fusion” are used interchangeably herein. These terms refer to the joining together of two or more chemical elements or components, by whatever means including chemical conjugation or recombinant means. For example, a promoter or enhancer is operably linked to a coding sequence if it affects the transcription of the sequence. Generally, “operably linked” means that the DNA sequences being linked are contiguous, and in reading phase or in-frame. An “in-frame fusion” refers to the joining of two or more open reading frames (ORFs) to form a continuous longer ORF, in a manner that maintains the correct reading frame of the original ORFs. Thus, the resulting recombinant fusion protein is a single protein containing two or more segments that correspond to polypeptides encoded by the original ORFs (which segments are not normally so joined in nature).

In the context of polypeptides, a “linear sequence” or a “sequence” is an order of amino acids in a polypeptide in an amino to carboxyl terminus direction in which residues that neighbor each other in the sequence are contiguous in the primary structure of the polypeptide. A “partial sequence” is a linear sequence of part of a polypeptide that is known to comprise additional residues in one or both directions.

“Heterologous” means derived from a genotypically distinct entity from the rest of the entity to which it is being compared. For example, a glycine rich sequence removed from its native coding sequence and operatively linked to a coding sequence other than the native sequence is a heterologous glycine rich sequence. The term “heterologous” as applied to a polynucleotide, a polypeptide, means that the polynucleotide or polypeptide is derived from a genotypically distinct entity from that of the rest of the entity to which it is being compared.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “complement of a polynucleotide” denotes a polynucleotide molecule having a complementary base sequence and reverse orientation as compared to a reference sequence, such that it could hybridize with a reference sequence with complete fidelity.

“Recombinant” as applied to a polynucleotide means that the polynucleotide is the product of various combinations of in vitro cloning, restriction and/or ligation steps, and other procedures that result in a construct that can potentially be expressed in a host cell.

The terms “gene” or “gene fragment” are used interchangeably herein. They refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

“Homology” or “homologous” refers to sequence similarity or interchangeability between two or more polynucleotide sequences or two or more polypeptide sequences. When using a program such as BestFit to determine sequence identity, similarity or homology between two different amino acid sequences, the default settings may be used, or an appropriate scoring matrix, such as blosum45 or blosum80, may be selected to optimize identity, similarity or homology scores. Preferably, polynucleotides that are homologous are those which hybridize under stringent conditions as defined herein and have at least 70%, preferably at least 80%, more preferably at least 90%, more preferably 95%, more preferably 97%, more preferably 98%, and even more preferably 99% sequence identity to those sequences.

“Ligation” refers to the process of forming phosphodiester bonds between two nucleic acid fragments or genes, linking them together. To ligate the DNA fragments or genes together, the ends of the DNA must be compatible with each other. In some cases, the ends will be directly compatible after endonuclease digestion. However, it may be necessary to first convert the staggered ends commonly produced after endonuclease digestion to blunt ends to make them compatible for ligation.

The terms “stringent conditions” or “stringent hybridization conditions” includes reference to conditions under which a polynucleotide will hybridize to its target sequence, to a detectably greater degree than other sequences (e.g., at least 2-fold over background). Generally, stringency of hybridization is expressed, in part, with reference to the temperature and salt concentration under which the wash step is carried out. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short polynucleotides (e.g., 10 to 50 nucleotides) and at least about 60° C. for long polynucleotides (e.g., greater than 50 nucleotides)—for example, “stringent conditions” can include hybridization in 50% formamide, 1 M NaCl, 1% SDS at 37° C., and three washes for 15 min each in 0.1×SSC/1% SDS at 60° C. to 65° C. Alternatively, temperatures of about 65° C., 60° C., 55° C., or 42° C. may be used. SSC concentration may be varied from about 0.1 to 2×SSC, with SDS being present at about 0.1%. Such wash temperatures are typically selected to be about 5° C. to 20° C. lower than the thermal melting point for the specific sequence at a defined ionic strength and pH. The Tm is the temperature (under defined ionic strength and pH) at which 50% of the target sequence hybridizes to a perfectly matched probe. An equation for calculating Tm and conditions for nucleic acid hybridization are well known and can be found in Sambrook, J. et al. (1989) Molecular Cloning: A Laboratory Manual, 2nd ed., vol. 1-3, Cold Spring Harbor Press, Plainview N.Y.; specifically see volume 2 and chapter 9. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity may be measured over the length of an entire defined polynucleotide sequence, or may be measured over a shorter length, for example, over the length of a fragment taken from a larger, defined polynucleotide sequence, for instance, a fragment of at least 45, at least 60, at least 90, at least 120, at least 150, at least 210 or at least 450 contiguous residues. Such lengths are exemplary only, and it is understood that any fragment length supported by the sequences shown herein, in the tables, figures or Sequence Listing, may be used to describe a length over which percentage identity may be measured.

“Percent (%) amino acid sequence identity,” with respect to the polypeptide sequences identified herein, is defined as the percentage of amino acid residues in a query sequence that are identical with the amino acid residues of a second, reference polypeptide sequence or a portion thereof, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Percent identity may be measured over the length of an entire defined polypeptide sequence, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The term “non-repetitiveness” as used herein in the context of a polypeptide refers to a lack or limited degree of internal homology in a peptide or polypeptide sequence. The term “substantially non-repetitive” can mean, for example, that there are few or no instances of four contiguous amino acids in the sequence that are identical amino acid types or that the polypeptide has a subsequence score (defined infra) of 10 or less or that there isn't a pattern in the order, from N- to C-terminus, of the sequence motifs that constitute the polypeptide sequence. The term “repetitiveness” as used herein in the context of a polypeptide refers to the degree of internal homology in a peptide or polypeptide sequence. In contrast, a “repetitive” sequence may contain multiple identical copies of short amino acid sequences. For instance, a polypeptide sequence of interest may be divided into n-mer sequences and the number of identical sequences can be counted. Highly repetitive sequences contain a large fraction of identical sequences while non-repetitive sequences contain few identical sequences. In the context of a polypeptide, a sequence can contain multiple copies of shorter sequences of defined or variable length, or motifs, in which the motifs themselves have non-repetitive sequences, rendering the full-length polypeptide substantially non-repetitive. The length of polypeptide within which the non-repetitiveness is measured can vary from 3 amino acids to about 200 amino acids, about from 6 to about 50 amino acids, or from about 9 to about 14 amino acids. “Repetitiveness” used in the context of polynucleotide sequences refers to the degree of internal homology in the sequence such as, for example, the frequency of identical nucleotide sequences of a given length. Repetitiveness can, for example, be measured by analyzing the frequency of identical sequences.

A “vector” is a nucleic acid molecule, preferably self-replicating in an appropriate host, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

“Serum degradation resistance,” as applied to a polypeptide, refers to the ability of the polypeptides to withstand degradation in blood or components thereof, which typically involves proteases in the serum or plasma. The serum degradation resistance can be measured by combining the protein with human (or mouse, rat, monkey, as appropriate) serum or plasma, typically for a range of days (e.g. 0.25, 0.5, 1, 2, 4, 8, 16 days), typically at about 37° C. The samples for these time points can be run on a Western blot assay and the protein is detected with an antibody. The antibody can be to a tag in the protein. If the protein shows a single band on the western, where the protein's size is identical to that of the injected protein, then no degradation has occurred. In this exemplary method, the time point where 50% of the protein is degraded, as judged by Western blots or equivalent techniques, is the serum degradation half-life or “serum half-life” of the protein.

The term “t½” as used herein means the terminal half-life calculated as ln(2)/Kₑₗ. Kₑₗ is the terminal elimination rate constant calculated by linear regression of the terminal linear portion of the log concentration vs. time curve. Half-life typically refers to the time required for half the quantity of an administered substance deposited in a living organism to be metabolized or eliminated by normal biological processes. The terms “t½”, “terminal half-life”, “elimination half-life” and “circulating half-life” are used interchangeably herein.

“Apparent Molecular Weight Factor” or “Apparent Molecular Weight” are related terms referring to a measure of the relative increase or decrease in apparent molecular weight exhibited by a particular amino acid sequence. The Apparent Molecular Weight is determined using size exclusion chromatography (SEC) and similar methods compared to globular protein standards and is measured in “apparent kD” units. The Apparent Molecular Weight Factor is the ratio between the Apparent Molecular Weight and the actual molecular weight; the latter predicted by adding, based on amino acid composition, the calculated molecular weight of each type of amino acid in the composition.

The “hydrodynamic radius” or “Stokes radius” is the effective radius (Rh in nm) of a molecule in a solution measured by assuming that it is a body moving through the solution and resisted by the solution's viscosity. In the embodiments of the invention, the hydrodynamic radius measurements of the XTEN fusion proteins correlate with the ‘Apparent Molecular Weight Factor’, which is a more intuitive measure. The “hydrodynamic radius” of a protein affects its rate of diffusion in aqueous solution as well as its ability to migrate in gels of macromolecules. The hydrodynamic radius of a protein is determined by its molecular weight as well as by its structure, including shape and compactness. Methods for determining the hydrodynamic radius are well known in the art, such as by the use of size exclusion chromatography (SEC), as described in U.S. Pat. Nos. 6,406,632 and 7,294,513. Most proteins have globular structure, which is the most compact three-dimensional structure a protein can have with the smallest hydrodynamic radius. Some proteins adopt a random and open, unstructured, or ‘linear’ conformation and as a result have a much larger hydrodynamic radius compared to typical globular proteins of similar molecular weight.

“Physiological conditions” refer to a set of conditions in a living host as well as in vitro conditions, including temperature, salt concentration, pH, that mimic those conditions of a living subject. A host of physiologically relevant conditions for use in in vitro assays have been established. Generally, a physiological buffer contains a physiological concentration of salt and is adjusted to a neutral pH ranging from about 6.5 to about 7.8, and preferably from about 7.0 to about 7.5. A variety of physiological buffers is listed in Sambrook et al. (1989). Physiologically relevant temperature ranges from about 25° C. to about 38° C., and preferably from about 35° C. to about 37° C.

A “reactive group” is a chemical structure that can be coupled to a second reactive group. Examples for reactive groups are amino groups, carboxyl groups, sulfhydryl groups, hydroxyl groups, aldehyde groups, azide groups. Some reactive groups can be activated to facilitate coupling with a second reactive group. Non-limiting examples for activation are the reaction of a carboxyl group with carbodiimide, the conversion of a carboxyl group into an activated ester, or the conversion of a carboxyl group into an azide function.

“Controlled release agent”, “slow release agent”, “depot formulation” or “sustained release agent” are used interchangeably to refer to an agent capable of extending the duration of release of a polypeptide of the invention relative to the duration of release when the polypeptide is administered in the absence of agent. Different embodiments of the present invention may have different release rates, resulting in different therapeutic amounts.

The terms “antigen”, “target antigen” or “immunogen” are used interchangeably herein to refer to the structure or binding determinant that an antibody fragment or an antibody fragment-based therapeutic binds to or has specificity against.

The term “payload” as used herein refers to a protein or peptide sequence that has biological or therapeutic activity; the counterpart to the pharmacophore of small molecules. Examples of payloads include, but are not limited to, cytokines, enzymes, hormones and blood and growth factors. Payloads can further comprise genetically fused or chemically conjugated moieties such as chemotherapeutic agents, antiviral compounds, toxins, or contrast agents. These conjugated moieties can be joined to the rest of the polypeptide via a linker that may be cleavable or non-cleavable.

The term “antagonist”, as used herein, includes any molecule that partially or fully blocks, inhibits, or neutralizes a biological activity of a native polypeptide disclosed herein. Methods for identifying antagonists of a polypeptide may comprise contacting a native polypeptide with a candidate antagonist molecule and measuring a detectable change in one or more biological activities normally associated with the native polypeptide. In the context of the present invention, antagonists may include proteins, nucleic acids, carbohydrates, antibodies or any other molecules that decrease the effect of a biologically active protein.

The term “agonist” is used in the broadest sense and includes any molecule that mimics a biological activity of a native polypeptide disclosed herein. Suitable agonist molecules specifically include agonist antibodies or antibody fragments, fragments or amino acid sequence variants of native polypeptides, peptides, small organic molecules, etc. Methods for identifying agonists of a native polypeptide may comprise contacting a native polypeptide with a candidate agonist molecule and measuring a detectable change in one or more biological activities normally associated with the native polypeptide.

“Activity” for the purposes herein refers to an action or effect of a component of a fusion protein consistent with that of the corresponding native biologically active protein, wherein “biological activity” refers to an in vitro or in vivo biological function or effect, including but not limited to receptor binding, antagonist activity, agonist activity, or a cellular or physiologic response.

As used herein, “treatment” or “treating,” or “palliating” or “ameliorating” is used interchangeably herein. These terms refer to an approach for obtaining beneficial or desired results including but not limited to a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the pediatric subject, notwithstanding that the subject may still be afflicted with the underlying disorder. For prophylactic benefit, the compositions may be administered to a pediatric subject at risk of developing a particular disease, or to a pediatric subject reporting one or more of the physiological symptoms of a disease, even though a diagnosis of this disease may not have been made.

A “therapeutic effect”, as used herein, refers to a physiologic effect, including but not limited to the cure, mitigation, amelioration, or prevention of disease in humans or other animals, or to otherwise enhance physical or mental wellbeing of humans or animals, caused by a fusion polypeptide of the invention other than the ability to induce the production of an antibody against an antigenic epitope possessed by the biologically active protein. Determination of a therapeutically effective amount is well within the capability of those skilled in the art, especially in light of the detailed disclosure provided herein.

The terms “therapeutically effective amount” and “therapeutically effective dose”, as used herein, refers to an amount of a biologically active protein, either alone or as a part of a fusion protein composition, that is capable of having any detectable, beneficial effect on any symptom, aspect, measured parameter or characteristics of a disease state or condition when administered in one or repeated doses to a pediatric subject. Such effect need not be absolute to be beneficial.

A “pharmaceutically acceptable carrier” refers to an ingredient in a pharmaceutical composition, other than an active ingredient, which is nontoxic to a pediatric subject. A pharmaceutically acceptable carrier includes, but is not limited to, a buffer, excipient, stabilizer, or preservative.

The term “therapeutically effective dose regimen”, as used herein, refers to a schedule for consecutively administered doses of a biologically active protein, either alone or as a part of a fusion protein composition, wherein the doses are given in therapeutically effective amounts to result in sustained beneficial effect on any symptom, aspect, measured parameter or characteristics of a disease state or condition.

The term “pediatric patient”, “pediatric subject”, as used herein, refers to an individual who is not an adult. Pediatric patients include infants, children, and adolescents. In one embodiment, the children are pre-adolescent or pre-pubertal individuals. In another embodiment, the pediatric patient is a human patient.

### I). General Techniques

The practice of the present invention employs, unless otherwise indicated, conventional techniques of immunology, biochemistry, chemistry, molecular biology, microbiology, cell biology, genomics and recombinant DNA, which are within the skill of the art. See Sambrook, J. et al., “Molecular Cloning: A Laboratory Manual,” 3rd edition, Cold Spring Harbor Laboratory Press, 2001; “Current protocols in molecular biology”, F. M. Ausubel, et al. eds., 1987; the series “Methods in Enzymology,” Academic Press, San Diego, Calif.; “PCR 2: a practical approach”, M. J. MacPherson, B. D. Hames and G. R. Taylor eds., Oxford University Press, 1995; “Antibodies, a laboratory manual” Harlow, E. and Lane, D. eds., Cold Spring Harbor Laboratory, 1988; “Goodman & Gilman's The Pharmacological Basis of Therapeutics,” 11th Edition, McGraw-Hill, 2005; and Freshney, R. I., “Culture of Animal Cells: A Manual of Basic Technique,” 4th edition, John Wiley & Sons, Somerset, N J, 2000, the contents of which are incorporated in their entirety herein by reference.

### II). Growth Hormone

The present invention concerns an improved therapeutic regimen for treating pediatric growth hormone deficiency (PGHD) patients. In particular, the invention concerns methods for bolus dose administration of a hGH-XTEN fusion protein to a pediatric patient with PGHD. Accordingly, in one aspect, the present invention concerns a method of treating human pediatric growth hormone deficiency (PGHD) in pediatric patients with a hGH-XTEN fusion protein.

(a) Growth Hormone Proteins

“Growth Hormone” or “GH” means a growth hormone protein and species and sequence variants thereof, and includes, but is not limited to, the 191 single-chain amino acid sequence of human GH. The GH can be the native, full-length protein or can be a truncated fragment or a sequence variant that retains at least a portion of the biological activity of the native protein. There are two known types of human GH (hereinafter “hGH”) derived from the pituitary gland: one having a molecular weight of about 22,129 daltons (22 kD hGH) and the other having a molecular weight of about 20,000 daltons (20 kD hGH). The 20 kD HGH has an amino acid sequence that corresponds to that of 22 kD hGH consisting of 191 amino acids except that 15 amino acid residues from the 32nd to the 46th of 22 kD hGH are missing. Some reports have shown that the 20 kD hGH has been found to exhibit lower risks and higher activity than 22 kD hGH. The invention contemplates use of the 22 kD, the 20 kD hGH, as well as species and sequence variants and truncated fragments thereof as being appropriate for use as a fusion partner with XTEN disclosed herein for hGH-XTEN compositions. The cloned gene for hGH has been expressed in a secreted form in Eschericha coli (U.S. Pat. No. 4,898,830; Chang, C. N., et al., Gene 55:189 [1987]) and its DNA and amino acid sequence has been reported (Goeddel, et al. Nature, 281:544 [1979]); Gray, et al., Gene 39: 247[1985]).

The invention contemplates inclusion in the hGH-XTEN compositions sequences with homology to GH sequences, sequence fragments that are natural, such as from humans and non-natural sequence variants which retain at least a portion of the biologic activity or biological function of GH and/or that are useful for preventing, treating, mediating, or ameliorating a GH-related disease, deficiency, disorder or condition in pediatric patients. In addition, native sequences homologous to human GH may be found by standard homology searching techniques, such as NCBI BLAST.

Effects of GH on the tissues of the body can generally be described as anabolic. Like most other protein hormones, native GH acts by interacting with a specific plasma membrane receptor, referred to as growth hormone receptor. GH acts on the liver and other tissues to stimulate production of IGF-I, which is responsible for the growth promoting effects of GH and also reflects the amount produced. IGF-I, in turn, has stimulatory effects on osteoblast and chondrocyte activity to promote bone growth. In one embodiment, the invention provides a hGH-XTEN that exhibits at least one of the properties of native GH hereinabove described herein.

In one embodiment, the GH incorporated into the subject compositions is a recombinant polypeptide with a sequence corresponding to a protein found in nature. In another embodiment, the GH is a sequence variant, fragment, homolog, or a mimetics of a natural sequence that retains at least a portion of the biological activity of the corresponding native GH. In one other embodiment, the GH is human GH comprising the following amino acid sequence: FPTIPLSRLFDNAMLRAHRLHQLAFDTYQEFEEAYIPKEQKYSFLQNPQTSLCFSES IPTPSNREETQQKSNLELLRISLLLIQSWLEPVQFLRSVFANSLVYGASDSNVYDLL KDLEEGIQTLMGRLEDGSPRTGQIFKQTYSKFDTNSHNDDALLKNYGLLYCFRKD MDKVETFLRIVQCRSVEGSCGF (SEQ ID NO:41). Any human GH sequences or homologous derivatives constructed by shuffling individual mutations between families that retain at least a portion of the biological activity of the native GH may be useful for the fusion proteins of this invention. GH that can be incorporated into a hGH-XTEN fusion protein can include a protein that exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO:41.

### III). Human Growth Hormone-XTEN Fusion Protein Compositions for Treating PGHD

The present invention concerns an improved therapeutic regimen for pediatric growth hormone deficiency (PGHD) therapy for pediatric patients. In particular, the invention concerns methods for bolus dose administration of hGH-XTEN fusion proteins to a pediatric patient with PGHD. In one aspect, the hGH fusion proteins suitable for use in the present invention comprise a human growth hormone polypeptide and one or more XTEN sequences as described herein, and as disclosed in Schellenberger et al. WO10/144502A2 and WO10/091122, which are incorporated herein by reference in their entirety.

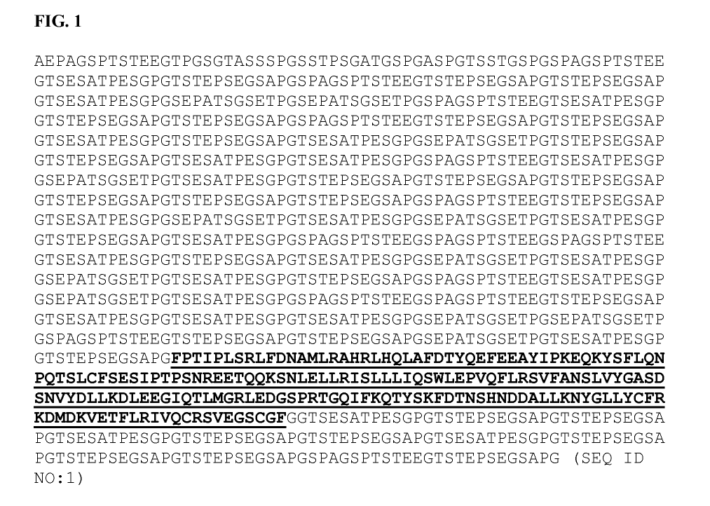

In one other aspect, the hGH-XTEN fusion proteins are isolated monomeric fusion proteins of GH comprising the full-length sequence or sequence variants of GH covalently linked to one or more extended recombinant polypeptides (“XTEN” or “XTENs”). In one embodiment, the hGH-XTEN fusion protein comprises an amino acid sequence shown in FIG. 1 (SEQ ID NO:1), or pharmacologically active variants thereof. In another embodiment, the hGH-XTEN fusion protein comprises an amino acid sequence selected from Table 1.

For example, the hGH-XTEN fusion protein VRS-317, is composed of recombinant human growth hormone (rhGH) and two recombinant polypeptides, referred to as XTEN as described in Schellenberger et al. (2009). Nat Biotechnol 27, 1186-90, Schellenberger et al. WO10/144502A2, and WO10/091122, each of which are incorporated herein by reference in their entirety. The XTEN domain, two unstructured hydrophilic chains of amino acids, provides half-life extension for rhGH. The molecular weight of VRS-317 is 118.9 kDa, with rhGH contributing 22.1 kDa and the remaining mass contributed by the XTEN construct. The mass ratio of rhGH to VRS-317 is therefore 1:5.37.

TABLE 1
Exemplary hGH-XTEN fusion proteins

| hGH- | | SEQ | | SEQ |
|---|---|---|---|---|
| XTEN | | ID | | ID |
| Name* | Amino Acid Sequence | NO: | DNA Nucleotide Sequence | NO: |
| AE912- | AEPAGSPTSTEEGTPGS | 1 | ATGGCTGAACCTGCTGGCTCTCCAACCTCCACT | 7 |
| hGH- | GTASSSPGSSTPSGATG | | GAGGAAGGTACCCCGGGTAGCGGTACTGCTTCT | |
| AE144 | SPGASPGTSSTGSPGSP | | TCCTCTCCAGGTAGCTCTACCCCTTCTGGTGCAA | |
| | AGSPTSTEEGTSESATP | | CCGGCTCTCCAGGTGCTTCTCCGGGCACCAGCT | |
| | ESGPGTSTEPSEGSAPG | | CTACCGGTTCTCCAGGTAGCCCGGCTGGCTCTC | |
| | SPAGSPTSTEEGTSTEPS | | CTACCTCTACTGAGGAAGGTACTTCTGAAAGCG | |
| | EGSAPGTSTEPSEGSAP | | CTACTCCTGAGTCTGGTCCAGGTACCTCTACTG | |
| | GTSESATPESGPGSEPA | | AACCGTCCGAAGGTAGCGCTCCAGGTAGCCCA | |
| | TSGSETPGSEPATSGSET | | GCAGGCTCTCCGACTTCCACTGAGGAAGGTACT | |
| | PGSPAGSPTSTEEGTSES | | TCTACTGAACCTTCCGAAGGCAGCGCACCAGGT | |
| | ATPESGPGTSTEPSEGS | | ACCTCTACTGAACCTTCTGAGGGCAGCGCTCCA | |
| | APGTSTEPSEGSAPGSP | | GGTACTTCTGAAAGCGCTACCCCGGAATCTGGC | |
| | AGSPTSTEEGTSTEPSE | | CCAGGTAGCGAACCGGCTACTTCTGGTTCTGAA | |
| | GSAPGTSTEPSEGSAPG | | ACCCCAGGTAGCGAACCGGCTACCTCCGGTTCT | |
| | TSESATPESGPGTSTEPS | | GAAACTCCAGGTAGCCCGGCAGGCTCTCCGACC | |
| | EGSAPGTSESATPESGP | | TCTACTGAGGAAGGTACTTCTGAAAGCGCAACC | |
| | GSEPATSGSETPGTSTEP | | CCGGAGTCCGGCCCAGGTACCTCTACCGAACCG | |
| | SEGSAPGTSTEPSEGSA | | TCTGAGGGCAGCGCACCAGGTACTTCTACCGAA | |
| | PGTSESATPESGPGTSES | | CCGTCCGAGGGTAGCGCACCAGGTAGCCCAGC | |
| | ATPESGPGSPAGSPTST | | AGGTTCTCCTACCTCCACCGAGGAAGGTACTTC | |
| | EEGTSESATPESGPGSEP | | TACCGAACCGTCCGAGGGTAGCGCACCAGGTA | |
| | ATSGSETPGTSESATPES | | CCTCTACTGAACCTTCTGAGGGCAGCGCTCCAG | |
| | GPGTSTEPSEGSAPGTS | | GTACTTCTGAAAGCGCTACCCCGGAGTCCGGTC | |
| | TEPSEGSAPGTSTEPSEG | | CAGGTACTTCTACTGAACCGTCCGAAGGTAGCG | |
| | SAPGTSTEPSEGSAPGT | | CACCAGGTACTTCTGAAAGCGCAACCCCTGAAT | |
| | STEPSEGSAPGTSTEPSE | | CCGGTCCAGGTAGCGAACCGGCTACTTCTGGCT | |
| | GSAPGSPAGSPTSTEEG | | CTGAGACTCCAGGTACTTCTACCGAACCGTCCG | |
| | TSTEPSEGSAPGTSESAT | | AAGGTAGCGCACCAGGTACTTCTACTGAACCGT | |
| | PESGPGSEPATSGSETP | | CTGAAGGTAGCGCACCAGGTACTTCTGAAAGCG | |
| | GTSESATPESGPGSEPA | | CAACCCCGGAATCCGGCCCAGGTACCTCTGAAA | |
| | TSGSETPGTSESATPESG | | GCGCAACCCCGGAGTCCGGCCCAGGTAGCCCTG | |
| | PGTSTEPSEGSAPGTSES | | CTGGCTCTCCAACCTCCACCGAAGAAGGTACCT | |
| | ATPESGPGSPAGSPTST | | CTGAAAGCGCAACCCCTGAATCCGGCCCAGGTA | |
| | EEGSPAGSPTSTEEGSP | | GCGAACCGGCAACCTCCGGTTCTGAAACCCCAG | |
| | AGSPTSTEEGTSESATP | | GTACCTCTGAAAGCGCTACTCCGGAGTCTGGCC | |
| | ESGPGTSTEPSEGSAPG | | CAGGTACCTCTACTGAACCGTCTGAGGGTAGCG | |
| | TSESATPESGPGSEPATS | | CTCCAGGTACTTCTACTGAACCGTCCGAAGGTA | |
| | GSETPGTSESATPESGP | | GCGCACCAGGTACTTCTACCGAACCGTCCGAAG | |
| | GSEPATSGSETPGTSES | | GCAGCGCTCCAGGTACCTCTACTGAACCTTCCG | |
| | ATPESGPGTSTEPSEGS | | AGGGCAGCGCTCCAGGTACCTCTACCGAACCTT | |
| | APGSPAGSPTSTEEGTS | | CTGAAGGTAGCGCACCAGGTACTTCTACCGAAC | |
| | ESATPESGPGSEPATSG | | CGTCCGAGGGTAGCGCACCAGGTAGCCCAGCA | |
| | SETPGTSESATPESGPGS | | GGTTCTCCTACCTCCACCGAGGAAGGTACTTCT | |
| | PAGSPTSTEEGSPAGSP | | ACCGAACCGTCCGAGGGTAGCGCACCAGGTAC | |
| | TSTEEGTSTEPSEGSAP | | CTCTGAAAGCGCAACTCCTGAGTCTGGCCCAGG | |
| | GTSESATPESGPGTSES | | TAGCGAACCTGCTACCTCCGGCTCTGAGACTCC | |
| | ATPESGPGTSESATPES | | AGGTACCTCTGAAAGCGCAACCCCGGAATCTGG | |
| | GPGSEPATSGSETPGSE | | TCCAGGTAGCGAACCTGCAACCTCTGGCTCTGA | |
| | PATSGSETPGSPAGSPTS | | AACCCCAGGTACCTCTGAAAGCGCTACTCCTGA | |
| | TEEGTSTEPSEGSAPGT | | ATCTGGCCCAGGTACTTCTACTGAACCGTCCGA | |
| | STEPSEGSAPGSEPATS | | GGGCAGCGCACCAGGTACTTCTGAAAGCGCTAC | |
| | GSETPGTSESATPESGP | | TCCTGAGTCCGGCCCAGGTAGCCCGGCTGGCTC | |
| | GTSTEPSEGSAPGFPTIP | | TCCGACTTCCACCGAGGAAGGTAGCCCGGCTGG | |
| | LSRLFDNAMLRAHRLH | | CTCTCCAACTTCTACTGAAGAAGGTAGCCCGGC | |
| | QLAFDTYQEFEEAYIPK | | AGGCTCTCCGACCTCTACTGAGGAAGGTACTTC | |
| | EQKYSFLQNPQTSLCFS | | TGAAAGCGCAACCCCGGAGTCCGGCCCAGGTA | |
| | ESIPTPSNREETQQKSNL | | CCTCTACCGAACCGTCTGAGGGCAGCGCACCAG | |
| | ELLRISELLIQSWLEPVQ | | GTACCTCTGAAAGCGCAACTCCTGAGTCTGGCC | |
| | FLRSVFANSLVYGASDS | | CAGGTAGCGAACCTGCTACCTCCGGCTCTGAGA | |
| | NVYDLLKDLEEGIQTL | | CTCCAGGTACCTCTGAAAGCGCAACCCCGGAAT | |
| | MGRLEDGSPRTGQIFK | | CTGGTCCAGGTAGCGAACCTGCAACCTCTGGCT | |
| | QTYSKFDTNSHNDDAL | | CTGAAACCCCAGGTACCTCTGAAAGCGCTACTC | |
| | LKNYGLLYCFRKDMD | | CTGAATCTGGCCCAGGTACTTCTACTGAACCGT | |
| | KVETFLRIVQCRSVEGS | | CCGAGGGCAGCGCACCAGGTAGCCCTGCTGGCT | |
| | CGFGGTSESATPESGPG | | CTCCAACCTCCACCGAAGAAGGTACCTCTGAAA | |
| | TSTEPSEGSAPGTSTEPS | | GCGCAACCCCTGAATCCGGCCCAGGTAGCGAA | |
| | EGSAPGTSESATPESGP | | CCGGCAACCTCCGGTTCTGAAACCCCAGGTACT | |
| | GTSTEPSEGSAPGTSTEP | | TCTGAAAGCGCTACTCCTGAGTCCGGCCCAGGT | |
| | SEGSAPGTSESATPESG | | AGCCCGGCTGGCTCTCCGACTTCCACCGAGGAA | |
| | PGTSTEPSEGSAPGTSTE | | GGTAGCCCGGCTGGCTCTCCAACTTCTACTGAA | |
| | PSEGSAPGTSTEPSEGS | | GAAGGTACTTCTACCGAACCTTCCGAGGGCAGC | |
| | APGSPAGSPTSTEEGTS | | GCACCAGGTACTTCTGAAAGCGCTACCCCTGAG | |
| | TEPSEGSAPG | | TCCGGCCCAGGTACTTCTGAAAGCGCTACTCCT | |
| | | | GAATCCGGTCCAGGTACTTCTGAAAGCGCTACC | |
| | | | CCGGAATCTGGCCCAGGTAGCGAACCGGCTACT | |
| | | | TCTGGTTCTGAAACCCCAGGTAGCGAACCGGCT | |
| | | | ACCTCCGGTTCTGAAACTCCAGGTAGCCCAGCA | |
| | | | GGCTCTCCGACTTCCACTGAGGAAGGTACTTCT | |
| | | | ACTGAACCTTCCGAAGGCAGCGCACCAGGTACC | |
| | | | TCTACTGAACCTTCTGAGGGCAGCGCTCCAGGT | |
| | | | AGCGAACCTGCAACCTCTGGCTCTGAAACCCCA | |
| | | | GGTACCTCTGAAAGCGCTACTCCTGAATCTGGC | |
| | | | CCAGGTACTTCTACTGAACCGTCCGAGGGCAGC | |
| | | | GCACCAGGTTTTCCGACTATTCCGCTGTCTCGTC | |
| | | | TGTTTGATAATGCTATGCTGCGTGCGCACCGTC | |
| | | | TGCACCAGCTGGCCTTTGATACTTACCAGGAAT | |
| | | | TTGAAGAAGCcTACATTCCTAAAGAGCAGAAGT | |
| | | | ACTCTTTCCTGCAAAACCCACAGACTTCTCTCTG | |
| | | | CTTCAGCGAATCTATTCCGACGCCTTCCAATCG | |
| | | | CGAGGAAACTCAGCAAAAGTCCAATCTGGAAC | |
| | | | TACTCCGCATTTCTCTGCTTCTGATTCAGAGCTG | |
| | | | GCTAGAACCAGTGCAATTTCTGCGTTCCGTCTT | |
| | | | CGCCAATAGCCTAGTTTATGGCGCATCCGACAG | |
| | | | CAACGTATACGATCTCCTGAAAGATCTCGAGGA | |
| | | | AGGCATTCAGACCCTGATGGGTCGTCTCGAGGA | |
| | | | TGGCTCTCCGCGTACTGGTCAGATCTTCAAGCA | |
| | | | GACTTACTCTAAATTTGATACTAACAGCCACAA | |
| | | | TGACGATGCGCTTCTAAAAAACTATGGTCTGCT | |
| | | | GTATTGTTTTCGTAAAGATATGGACAAAGTTGA | |
| | | | AACCTTCCTGCGTATTGTTCAGTGTCGTTCCGTT | |
| | | | GAGGGCAGCTGTGGTTTCTAAGGTGGTAGCGAA | |
| | | | CCGGCAACTTCCGGCTCTGAAACCCCAGGTACT | |
| | | | TCTGAAAGCGCTACTCCTGAGTCTGGCCCAGGT | |
| | | | AGCGAACCTGCTACCTCTGGCTCTGAAACCCCA | |
| | | | GGTAGCCCGGCAGGCTCTCCGACTTCCACCGAG | |
| | | | GAAGGTACCTCTACTGAACCTTCTGAGGGTAGC | |
| | | | GCTCCAGGTAGCGAACCGGCAACCTCTGGCTCT | |
| | | | GAAACCCCAGGTAGCGAACCTGCTACCTCCGGC | |
| | | | TCTGAAACTCCAGGTAGCGAACCGGCTACTTCC | |
| | | | GGTTCTGAAACTCCAGGTACCTCTACCGAACCT | |
| | | | TCCGAAGGCAGCGCACCAGGTACTTCTGAAAGC | |
| | | | GCAACCCCTGAATCCGGTCCAGGTAGCGAACCG | |
| | | | GCTACTTCTGGCTCTGAGACTCCAGGTACTTCT | |
| | | | ACCGAACCGTCCGAAGGTAGCGCACCA | |
| AM864- | GGSPGTSTEPSEGSAPG | 2 | ggtGGGTCTCCAGGTACTTCTACTGAACCGTCTG | 8 |
| hGH | SEPATSGSETPGSPAGSP | | AAGGCAGCGCACCAGGTAGCGAACCGGCTACT | |
| | TSTEEGSTSSTAESPGPG | | TCCGGTTCTGAAACCCCAGGTAGCCCAGCAGGT | |
| | TSTPESGSASPGSTSESP | | TCTCCAACTTCTACTGAAGAAGGTTCTACCAGC | |
| | SGTAPGSTSESPSGTAP | | TCTACCGCAGAATCTCCTGGTCCAGGTACCTCT | |
| | GTSTPESGSASPGTSTPE | | ACTCCGGAAAGCGGCTCTGCATCTCCAGGTTCT | |
| | SGSASPGSEPATSGSETP | | ACTAGCGAATCTCCTTCTGGCACTGCACCAGGT | |
| | GTSESATPESGPGSPAG | | TCTACTAGCGAATCCCCGTCTGGTACTGCTCCA | |
| | SPTSTEEGTSTEPSEGSA | | GGTACTTCTACTCCTGAAAGCGGTTCCGCTTCTC | |
| | PGTSESATPESGPGTSTE | | CAGGTACCTCTACTCCGGAAAGCGGTTCTGCAT | |
| | PSEGSAPGTSTEPSEGS | | CTCCAGGTAGCGAACCGGCAACCTCCGGCTCTG | |
| | APGSPAGSPTSTEEGTS | | AAACCCCAGGTACCTCTGAAAGCGCTACTCCTG | |
| | TEPSEGSAPGTSTEPSEG | | AATCCGGCCCAGGTAGCCCGGCAGGTTCTCCGA | |
| | SAPGTSESATPESGPGT | | CTTCCACTGAGGAAGGTACCTCTACTGAACCTT | |
| | SESATPESGPGTSTEPSE | | CTGAGGGCAGCGCTCCAGGTACTTCTGAAAGCG | |
| | GSAPGTSTEPSEGSAPG | | CTACCCCGGAGTCCGGTCCAGGTACTTCTACTG | |
| | TSESATPESGPGTSTEPS | | AACCGTCCGAAGGTAGCGCACCAGGTACTTCTA | |
| | EGSAPGSEPATSGSETP | | CCGAACCGTCCGAGGGTAGCGCACCAGGTAGC | |
| | GSPAGSPTSTEEGSSTPS | | CCAGCAGGTTCTCCTACCTCCACCGAGGAAGGT | |
| | GATGSPGTPGSGTASSS | | ACTTCTACCGAACCGTCCGAGGGTAGCGCACCA | |
| | PGSSTPSGATGSPGTST | | GGTACTTCTACCGAACCTTCCGAGGGCAGCGCA | |
| | EPSEGSAPGTSTEPSEGS | | CCAGGTACTTCTGAAAGCGCTACCCCTGAGTCC | |
| | APGSEPATSGSETPGSP | | GGCCCAGGTACTTCTGAAAGCGCTACTCCTGAA | |
| | AGSPTSTEEGSPAGSPT | | TCCGGTCCAGGTACCTCTACTGAACCTTCCGAA | |
| | STEEGTSTEPSEGSAPG | | GGCAGCGCTCCAGGTACCTCTACCGAACCGTCC | |
| | ASASGAPSTGGTSESAT | | GAGGGCAGCGCACCAGGTACTTCTGAAAGCGC | |
| | PESGPGSPAGSPTSTEE | | AACCCCTGAATCCGGTCCAGGTACTTCTACTGA | |
| | GSPAGSPTSTEEGSTSST | | ACCTTCCGAAGGTAGCGCTCCAGGTAGCGAACC | |
| | AESPGPGSTSESPSGTAP | | TGCTACTTCTGGTTCTGAAACCCCAGGTAGCCC | |
| | GTSPSGESSTAPGTPGS | | GGCTGGCTCTCCGACCTCCACCGAGGAAGGTAG | |
| | GTASSSPGSSTPSGATG | | CTCTACCCCGTCTGGTGCTACTGGTTCTCCAGGT | |
| | SPGSSPSASTGTGPGSEP | | ACTCCGGGCAGCGGTACTGCTTCTTCCTCTCCA | |
| | ATSGSETPGTSESATPES | | GGTAGCTCTACCCCTTCTGGTGCTACTGGCTCTC | |
| | GPGSEPATSGSETPGST | | CAGGTACCTCTACCGAACCGTCCGAGGGTAGCG | |
| | SSTAESPGPGSTSSTAES | | CACCAGGTACCTCTACTGAACCGTCTGAGGGTA | |
| | PGPGTSPSGESSTAPGSE | | GCGCTCCAGGTAGCGAACCGGCAACCTCCGGTT | |
| | PATSGSETPGSEPATSG | | CTGAAACTCCAGGTAGCCCTGCTGGCTCTCCGA | |
| | SETPGTSTEPSEGSAPGS | | CTTCTACTGAGGAAGGTAGCCCGGCTGGTTCTC | |
| | TSSTAESPGPGTSTPESG | | CGACTTCTACTGAGGAAGGTACTTCTACCGAAC | |
| | SASPGSTSESPSGTAPGT | | CTTCCGAAGGTAGCGCTCCAGGTGCAAGCGCAA | |
| | STEPSEGSAPGTSTEPSE | | GCGGCGCGCCAAGCACGGGAGGTACTTCTGAA | |
| | GSAPGTSTEPSEGSAPG | | AGCGCTACTCCTGAGTCCGGCCCAGGTAGCCCG | |
| | SSTPSGATGSPGSSPSAS | | GCTGGCTCTCCGACTTCCACCGAGGAAGGTAGC | |
| | TGTGPGASPGTSSTGSP | | CCGGCTGGCTCTCCAACTTCTACTGAAGAAGGT | |
| | GSEPATSGSETPGTSES | | TCTACCAGCTCTACCGCTGAATCTCCTGGCCCA | |
| | ATPESGPGSPAGSPTST | | GGTTCTACTAGCGAATCTCCGTCTGGCACCGCA | |
| | EEGSSTPSGATGSPGSSP | | CCAGGTACTTCCCCTAGCGGTGAATCTTCTACT | |
| | SASTGTGPGASPGTSST | | GCACCAGGTACCCCTGGCAGCGGTACCGCTTCT | |
| | GSPGTSESATPESGPGT | | TCCTCTCCAGGTAGCTCTACCCCGTCTGGTGCTA | |
| | STEPSEGSAPGTSTEPSE | | CTGGCTCTCCAGGTTCTAGCCCGTCTGCATCTAC | |
| | GSAPGFPTIPLSRLFDNA | | CGGTACCGGCCCAGGTAGCGAACCGGCAACCT | |
| | MLRAHRLHQLAFDTYQ | | CCGGCTCTGAAACTCCAGGTACTTCTGAAAGCG | |
| | EFEEAYIPKEQKYSFLQ | | CTACTCCGGAATCCGGCCCAGGTAGCGAACCGG | |
| | NPQTSLCFSESIPTPSNR | | CTACTTCCGGCTCTGAAACCCCAGGTTCCACCA | |
| | EETQQKSNLELLRISLL | | GCTCTACTGCAGAATCTCCGGGCCCAGGTTCTA | |
| | LIQSWLEPVQFLRSVFA | | CTAGCTCTACTGCAGAATCTCCGGGTCCAGGTA | |
| | NSLVYGASDSNVYDLL | | CTTCTCCTAGCGGCGAATCTTCTACCGCTCCAG | |
| | KDLEEGIQTLMGRLED | | GTAGCGAACCGGCAACCTCTGGCTCTGAAACTC | |
| | GSPRTGQIFKQTYSKFD | | CAGGTAGCGAACCTGCAACCTCCGGCTCTGAAA | |
| | TNSHNDDALLKNYGLL | | CCCCAGGTACTTCTACTGAACCTTCTGAGGGCA | |
| | YCFRKDMDKVETFLRI | | GCGCACCAGGTTCTACCAGCTCTACCGCAGAAT | |
| | VQCRSVEGSCGF | | CTCCTGGTCCAGGTACCTCTACTCCGGAAAGCG | |
| | | | GCTCTGCATCTCCAGGTTCTACTAGCGAATCTC | |
| | | | CTTCTGGCACTGCACCAGGTACTTCTACCGAAC | |
| | | | CGTCCGAAGGCAGCGCTCCAGGTACCTCTACTG | |
| | | | AACCTTCCGAGGGCAGCGCTCCAGGTACCTCTA | |
| | | | CCGAACCTTCTGAAGGTAGCGCACCAGGTAGCT | |
| | | | CTACTCCGTCTGGTGCAACCGGCTCCCCAGGTT | |
| | | | CTAGCCCGTCTGCTTCCACTGGTACTGGCCCAG | |
| | | | GTGCTTCCCCGGGCACCAGCTCTACTGGTTCTC | |
| | | | CAGGTAGCGAACCTGCTACCTCCGGTTCTGAAA | |
| | | | CCCCAGGTACCTCTGAAAGCGCAACTCCGGAGT | |
| | | | CTGGTCCAGGTAGCCCTGCAGGTTCTCCTACCT | |
| | | | CCACTGAGGAAGGTAGCTCTACTCCGTCTGGTG | |
| | | | CAACCGGCTCCCCAGGTTCTAGCCCGTCTGCTT | |
| | | | CCACTGGTACTGGCCCAGGTGCTTCCCCGGGCA | |
| | | | CCAGCTCTACTGGTTCTCCAGGTACCTCTGAAA | |
| | | | GCGCTACTCCGGAGTCTGGCCCAGGTACCTCTA | |
| | | | CTGAACCGTCTGAGGGTAGCGCTCCAGGTACTT | |
| | | | CTACTGAACCGTCCGAAGGTAGCGCACCAGGTT | |
| | | | TTCCGACTATTCCGCTGTCTCGTCTGTTTGATAA | |
| | | | TGCTATGCTGCGTGCGCACCGTCTGCACCAGCT | |
| | | | GGCCTTTGATACTTACCAGGAATTTGAAGAAGC | |
| | | | CTACATTCCTAAAGAGCAGAAGTACTCTTTCCTG | |
| | | | CAAAACCCACAGACTTCTCTCTGCTTCAGCGAA | |
| | | | TCTATTCCGACGCCTTCCAATCGCGAGGAAACT | |
| | | | CAGCAAAAGTCCAATCTGGAACTACTCCGCATT | |
| | | | TCTCTGCTTCTGATTCAGAGCTGGCTAGAACCA | |
| | | | GTGCAATTTCTGCGTTCCGTCTTCGCCAATAGCC | |
| | | | TAGTTTATGGCGCATCCGACAGCAACGTATACG | |
| | | | ATCTCCTGAAAGATCTCGAGGAAGGCATTCAGA | |
| | | | CCCTGATGGGTCGTCTCGAGGATGGCTCTCCGC | |
| | | | GTACTGGTCAGATCTTCAAGCAGACTTACTCTA | |
| | | | AATTTGATACTAACAGCCACAATGACGATGCGC | |
| | | | TTCTAAAAAACTATGGTCTGCTGTATTGTTTTCG | |
| | | | TAAAGATATGGACAAAGTTGAAACCTTCCTGCG | |
| | | | TATTGTTCAGTGTCGTTCCGTTGAGGGCAGCTG | |
| | | | TGGTTTC | |
| Y576- | GEGSGEGSEGEGSEGSG | 3 | GGTGAGGGTTCTGGCGAAGGTTCCGAAGGTGA | 9 |
| hGH | EGEGSEGSGEGEGGSE | | GGGCTCCGAAGGATCTGGCGAAGGTGAGGGTT | |
| | GSEGEGSEGSGEGEGG | | CCGAAGGTTCTGGCGAAGGTGAAGGCGGTTCTG | |
| | EGSGEGEGSGEGSEGE | | AGGGATCCGAAGGTGAAGGCTCCGAAGGATCT | |
| | GGGEGSEGEGSGEGGE | | GGCGAAGGTGAAGGTGGTGAAGGTTCTGGCGA | |
| | GEGSEGGSEGEGGSEG | | AGGTGAGGGATCTGGCGAAGGCTCTGAAGGTG | |
| | GEGEGSEGSGEGEGSE | | AAGGTGGTGGTGAAGGCTCTGAAGGTGAAGGA | |
| | GGSEGEGSEGGSEGEGS | | TCTGGTGAAGGTGAAGGTTCCGAAGGTTCTGGC | |
| | EGSGEGEGSEGSGEGE | | AGGCGGCTCCGAAGGTGAAGGCGGATCTGAAG | |
| | GSEGSGEGEGSEGSGEG | | GCGGCGAAGGTGAAGGTTCCGAAGGTTCTGGT | |
| | EGSEGGSEGEGGSEGSE | | GAAGGTGAAGGATCTGAAGGTGGCTCCGAAGG | |
| | GEGSGEGSEGEGGSEGS | | TGAAGGATCTGAAGGCGGTTCCGAAGGTGAGG | |
| | EGEGGGEGSEGEGSGE | | GCTCTGAAGGTTCTGGCGAAGGTGAAGGCTCTG | |
| | GSEGEGGSEGSEGEGGS | | AAGGATCTGGTGAAGGTGAAGGTTCCGAAGGT | |
| | EGSEGEGGEGSGEGEG | | TCTGGTGAAGGTGAAGGTTCCGAAGGTTCTGGC | |
| | SEGSGEGEGSGEGSEGE | | GAAGGTGAAGGTTCTGAAGGTGGCTCTGAAGG | |
| | GSEGSGEGEGSEGSGEG | | TGAAGGCGGCTCTGAAGGATCCGAAGGTGAAG | |
| | EGGSEGSEGEGSGEGSE | | GTGGCGAAGGATCTGGTGAAGGTGAAGGTTCT | |
| | GEGSEGSGEGEGSEGSG | | GAAGGTTCTGGCGAAGGTGAGGGTTCTGGCGA | |
| | EGEGGSEGSEGEGGSE | | AGGTTCCGAAGGTGAGGGCTCCGAAGGATCTG | |
| | GSEGEGGSEGSEGEGG | | GCGAAGGTGAGGGTTCCGAAGGTTCTGGCGAA | |
| | EGSGEGEGSEGSGEGE | | GGTGAAGGCGGTTCTGAGGGATCCGAAGGTGA | |
| | GSGEGSEGEGSEGSGEG | | GGGTTCTGGCGAAGGTTCCGAAGGTGAGGGCTC | |
| | EGSEGSGEGEGGSEGSE | | CGAAGGATCTGGCGAAGGTGAGGGTTCCGAAG | |
| | GEGSEGSGEGEGGEGS | | GTTCTGGCGAAGGTGAAGGCGGTTCTGAGGGAT | |
| | GEGEGSGEGSEGEGGG | | CCGAAGGTGAAGGCGGTTCTGAAGGTTCCGAA | |
| | EGSEGEGSEGSGEGEGS | | GGTGAAGGTGGCTCTGAGGGATCCGAAGGTGA | |
| | EGSGEGEGSEGGSEGE | | AGGTGGCGAAGGATCTGGTGAAGGTGAAGGTT | |
| | GGSEGSEGEGSEGGSEG | | CTGAAGGTTCTGGCGAAGGTGAGGGTTCTGGCG | |
| | EGSEGGSEGEGSEGSGE | | AAGGTTCCGAAGGTGAGGGCTCCGAAGGATCT | |
| | GEGSEGSGEGEGSGEGS | | GGCGAAGGTGAGGGTTCCGAAGGTTCTGGCGA | |
| | EGEGGSEGGEGEGSEG | | AGGTGAAGGCGGTTCTGAGGGATCCGAAGGTG | |
| | GSEGEGSEGGSEGEGG | | AAGGCTCCGAAGGATCTGGCGAAGGTGAAGGT | |
| | EGSGEGEGGGEGSEGE | | GGTGAAGGTTCTGGCGAAGGTGAGGGATCTGG | |
| | GSEGSGEGEGSGEGSEG | | CGAAGGCTCTGAAGGTGAAGGTGGTGGTGAAG | |
| | FPTIPLSRLFDNAMLRA | | GCTCTGAAGGTGAAGGTTCCGAAGGTTCTGGTG | |
| | HRLHQLAFDTYQEFEE | | AAGGTGAAGGTTCCGAAGGTTCTGGCGAAGGT | |
| | AYIPKEQKYSFLQNPQT | | GAAGGTTCTGAAGGTGGCTCTGAAGGTGAAGG | |
| | SLCFSESIPTPSNREETQ | | CGGCTCTGAAGGATCCGAAGGTGAAGGATCTG | |
| | QKSNLELLRISLLLIQS | | AAGGTGGCTCCGAAGGTGAAGGATCTGAAGGC | |
| | WLEPVQFLRSVFANSL | | GGTTCCGAAGGTGAGGGCTCTGAAGGTTCTGGC | |
| | VYGASDSNVYDLLKDL | | GAAGGTGAAGGCTCTGAAGGATCTGGTGAAGG | |
| | EEGIQTLMGRLEDGSPR | | TGAAGGATCTGGCGAAGGCTCCGAAGGTGAAG | |
| | TGQIFKQTYSKFDTNSH | | GCGGTTCTGAAGGTGGCGAAGGTGAAGGATCT | |
| | NDDALLKNYGLLYCFR | | GAAGGTGGTTCCGAAGGTGAGGGATCTGAAGG | |
| | KDMDKVETFLRIVQCR | | TGGCTCTGAAGGTGAAGGTGGCGAAGGTTCTGG | |
| | SVEGSCGF | | CGAAGGTGAAGGTGGAGGCGAAGGTTCTGAAG | |
| | | | GTGAAGGTTCCGAAGGTTCTGGTGAAGGTGAG | |
| | | | GGATCTGGCGAAGGTTCTGAAGGTTTTCCGACT | |
| | | | ATTCCGCTGTCTCGTCTGTTTGATAACGCTATGC | |
| | | | TGCGTGCGCACCGTCTGCACCAGCTGGCGTTCG | |
| | | | ACACTTACCAGGAATTTGAAGAAGCGTACATTC | |
| | | | CGAAGGAACAGAAGTACTCTTTCCTGCAAAACC | |
| | | | CGCAGACCTCCCTGTGCTTCAGCGAATCTATTC | |
| | | | CGACTCCGTCCAATCGTGAAGAAACTCAGCAAA | |
| | | | AGTCCAATCTGGAGCTGCTGCGCATCTCTCTGC | |
| | | | TGCTGATTCAGAGCTGGCTGGAGCCTGTTCAGT | |
| | | | TTCTGCGTTCCGTCTTCGCCAACAGCCTGGTTTA | |
| | | | TGGTGCTTCCGACAGCAACGTATACGATCTGCT | |
| | | | GAAAGATCTGGAAGAAGGCATTCAGACCCTGA | |
| | | | TGGGTCGTCTGGAAGATGGTTCTCCGCGTACTG | |
| | | | GTCAGATCTTCAAACAAACTTACTCCAAATTTG | |
| | | | ATACTAACAGCCATAACGACGATGCTCTGCTGA | |
| | | | AAAACTATGGTCTGCTGTATTGCTTCCGCAAGG | |
| | | | ATATGGACAAAGTTGAAACCTTCCTGCGTATTG | |
| | | | TGCAGTGTCGTTCCGTTGAGGGCAGCTGTGGTT | |
| | | | TC | |
| AE912- | AEPAGSPTSTEEGTPGS | 4 | ATGGCTGAACCTGCTGGCTCTCCAACCTCCACT | 10 |
| hGH | GTASSSPGSSTPSGATG | | GAGGAAGGTACCCCGGGTAGCGGTACTGCTTCT | |
| | SPGASPGTSSTGSPGSP | | TCCTCTCCAGGTAGCTCTACCCCTTCTGGTGCAA | |
| | AGSPTSTEEGTSESATP | | CCGGCTCTCCAGGTGCTTCTCCGGGCACCAGCT | |
| | ESGPGTSTEPSEGSAPG | | CTACCGGTTCTCCAGGTAGCCCGGCTGGCTCTC | |
| | SPAGSPTSTEEGTSTEPS | | CTACCTCTACTGAGGAAGGTACTTCTGAAAGCG | |
| | EGSAPGTSTEPSEGSAP | | CTACTCCTGAGTCTGGTCCAGGTACCTCTACTG | |
| | GTSESATPESGPGSEPA | | AACCGTCCGAAGGTAGCGCTCCAGGTAGCCCA | |
| | TSGSETPGSEPATSGSET | | GCAGGCTCTCCGACTTCCACTGAGGAAGGTACT | |
| | PGSPAGSPTSTEEGTSES | | TCTACTGAACCTTCCGAAGGCAGCGCACCAGGT | |
| | ATPESGPGTSTEPSEGS | | ACCTCTACTGAACCTTCTGAGGGCAGCGCTCCA | |
| | APGTSTEPSEGSAPGSP | | GGTACTTCTGAAAGCGCTACCCCGGAATCTGGC | |
| | AGSPTSTEEGTSTEPSE | | CCAGGTAGCGAACCGGCTACTTCTGGTTCTGAA | |
| | GSAPGTSTEPSEGSAPG | | ACCCCAGGTAGCGAACCGGCTACCTCCGGTTCT | |
| | TSESATPESGPGTSTEPS | | GAAACTCCAGGTAGCCCGGCAGGCTCTCCGACC | |
| | EGSAPGTSESATPESGP | | TCTACTGAGGAAGGTACTTCTGAAAGCGCAACC | |
| | GSEPATSGSETPGTSTEP | | CCGGAGTCCGGCCCAGGTACCTCTACCGAACCG | |
| | SEGSAPGTSTEPSEGSA | | TCTGAGGGCAGCGCACCAGGTACTTCTACCGAA | |
| | PGTSESATPESGPGTSES | | CCGTCCGAGGGTAGCGCACCAGGTAGCCCAGC | |
| | ATPESGPGSPAGSPTST | | AGGTTCTCCTACCTCCACCGAGGAAGGTACTTC | |
| | EEGTSESATPESGPGSEP | | TACCGAACCGTCCGAGGGTAGCGCACCAGGTA | |
| | ATSGSETPGTSESATPES | | CCTCTACTGAACCTTCTGAGGGCAGCGCTCCAG | |
| | GPGTSTEPSEGSAPGTS | | GTACTTCTGAAAGCGCTACCCCGGAGTCCGGTC | |
| | TEPSEGSAPGTSTEPSEG | | CAGGTACTTCTACTGAACCGTCCGAAGGTAGCG | |
| | SAPGTSTEPSEGSAPGT | | CACCAGGTACTTCTGAAAGCGCAACCCCTGAAT | |
| | STEPSEGSAPGTSTEPSE | | CCGGTCCAGGTAGCGAACCGGCTACTTCTGGCT | |
| | GSAPGSPAGSPTSTEEG | | CTGAGACTCCAGGTACTTCTACCGAACCGTCCG | |
| | TSTEPSEGSAPGTSESAT | | AAGGTAGCGCACCAGGTACTTCTACTGAACCGT | |
| | PESGPGSEPATSGSETP | | CTGAAGGTAGCGCACCAGGTACTTCTGAAAGCG | |
| | GTSESATPESGPGSEPA | | CAACCCCGGAATCCGGCCCAGGTACCTCTGAAA | |
| | TSGSETPGTSESATPESG | | GCGCAACCCCGGAGTCCGGCCCAGGTAGCCCTG | |
| | PGTSTEPSEGSAPGTSES | | CTGGCTCTCCAACCTCCACCGAAGAAGGTACCT | |
| | ATPESGPGSPAGSPTST | | CTGAAAGCGCAACCCCTGAATCCGGCCCAGGTA | |
| | EEGSPAGSPTSTEEGSP | | GCGAACCGGCAACCTCCGGTTCTGAAACCCCAG | |
| | AGSPTSTEEGTSESATP | | GTACCTCTGAAAGCGCTACTCCGGAGTCTGGCC | |
| | ESGPGTSTEPSEGSAPG | | CAGGTACCTCTACTGAACCGTCTGAGGGTAGCG | |
| | TSESATPESGPGSEPATS | | CTCCAGGTACTTCTACTGAACCGTCCGAAGGTA | |
| | GSETPGTSESATPESGP | | GCGCACCAGGTACTTCTACCGAACCGTCCGAAG | |
| | GSEPATSGSETPGTSES | | GCAGCGCTCCAGGTACCTCTACTGAACCTTCCG | |
| | ATPESGPGTSTEPSEGS | | AGGGCAGCGCTCCAGGTACCTCTACCGAACCTT | |
| | APGSPAGSPTSTEEGTS | | CTGAAGGTAGCGCACCAGGTACTTCTACCGAAC | |
| | ESATPESGPGSEPATSG | | CGTCCGAGGGTAGCGCACCAGGTAGCCCAGCA | |
| | SETPGTSESATPESGPGS | | GGTTCTCCTACCTCCACCGAGGAAGGTACTTCT | |
| | PAGSPTSTEEGSPAGSP | | ACCGAACCGTCCGAGGGTAGCGCACCAGGTAC | |
| | TSTEEGTSTEPSEGSAP | | CTCTGAAAGCGCAACTCCTGAGTCTGGCCCAGG | |
| | GTSESATPESGPGTSES | | TAGCGAACCTGCTACCTCCGGCTCTGAGACTCC | |
| | ATPESGPGTSESATPES | | AGGTACCTCTGAAAGCGCAACCCCGGAATCTGG | |
| | GPGSEPATSGSETPGSE | | TCCAGGTAGCGAACCTGCAACCTCTGGCTCTGA | |
| | PATSGSETPGSPAGSPTS | | AACCCCAGGTACCTCTGAAAGCGCTACTCCTGA | |
| | TEEGTSTEPSEGSAPGT | | ATCTGGCCCAGGTACTTCTACTGAACCGTCCGA | |
| | STEPSEGSAPGSEPATS | | GGGCAGCGCACCAGGTACTTCTGAAAGCGCTAC | |
| | GSETPGTSESATPESGP | | TCCTGAGTCCGGCCCAGGTAGCCCGGCTGGCTC | |
| | GTSTEPSEGSAPGFPTIP | | TCCGACTTCCACCGAGGAAGGTAGCCCGGCTGG | |
| | LSRLFDNAMLRAHRLH | | CTCTCCAACTTCTACTGAAGAAGGTAGCCCGGC | |
| | QLAFDTYQEFEEAYIPK | | AGGCTCTCCGACCTCTACTGAGGAAGGTACTTC | |
| | EQKYSFLQNPQTSLCFS | | TGAAAGCGCAACCCCGGAGTCCGGCCCAGGTA | |
| | ESIPTPSNREETQQKSNL | | CCTCTACCGAACCGTCTGAGGGCAGCGCACCAG | |
| | ELLRISLLLIQSWLEPVQ | | GTACCTCTGAAAGCGCAACTCCTGAGTCTGGCC | |
| | FLRSVFANSLVYGASDS | | CAGGTAGCGAACCTGCTACCTCCGGCTCTGAGA | |
| | NVYDLLKDLEEGIQTL | | CTCCAGGTACCTCTGAAAGCGCAACCCCGGAAT | |
| | MGRLEDGSPRTGQIFK | | CTGGTCCAGGTAGCGAACCTGCAACCTCTGGCT | |
| | QTYSKFDTNSHNDDAL | | CTGAAACCCCAGGTACCTCTGAAAGCGCTACTC | |
| | LKNYGLLYCFRKDMD | | CTGAATCTGGCCCAGGTACTTCTACTGAACCGT | |
| | KVETFLRIVQCRSVEGS | | CCGAGGGCAGCGCACCAGGTAGCCCTGCTGGCT | |
| | CGF | | CTCCAACCTCCACCGAAGAAGGTACCTCTGAAA | |
| | | | GCGCAACCCCTGAATCCGGCCCAGGTAGCGAA | |
| | | | CCGGCAACCTCCGGTTCTGAAACCCCAGGTACT | |
| | | | TCTGAAAGCGCTACTCCTGAGTCCGGCCCAGGT | |
| | | | AGCCCGGCTGGCTCTCCGACTTCCACCGAGGAA | |
| | | | GGTAGCCCGGCTGGCTCTCCAACTTCTACTGAA | |
| | | | GAAGGTACTTCTACCGAACCTTCCGAGGGCAGC | |
| | | | GCACCAGGTACTTCTGAAAGCGCTACCCCTGAG | |
| | | | TCCGGCCCAGGTACTTCTGAAAGCGCTACTCCT | |
| | | | GAATCCGGTCCAGGTACTTCTGAAAGCGCTACC | |
| | | | CCGGAATCTGGCCCAGGTAGCGAACCGGCTACT | |
| | | | TCTGGTTCTGAAACCCCAGGTAGCGAACCGGCT | |
| | | | ACCTCCGGTTCTGAAACTCCAGGTAGCCCAGCA | |
| | | | GGCTCTCCGACTTCCACTGAGGAAGGTACTTCT | |
| | | | ACTGAACCTTCCGAAGGCAGCGCACCAGGTACC | |
| | | | TCTACTGAACCTTCTGAGGGCAGCGCTCCAGGT | |
| | | | AGCGAACCTGCAACCTCTGGCTCTGAAACCCCA | |
| | | | GGTACCTCTGAAAGCGCTACTCCTGAATCTGGC | |
| | | | CCAGGTACTTCTACTGAACCGTCCGAGGGCAGC | |
| | | | GCACCAGGTTTTCCGACTATTCCGCTGTCTCGTC | |
| | | | TGTTTGATAATGCTATGCTGCGTGCGCACCGTC | |
| | | | TGCACCAGCTGGCCTTTGATACTTACCAGGAAT | |
| | | | TTGAAGAAGCcTACATTCCTAAAGAGCAGAAGT | |
| | | | ACTCTTTCCTGCAAAACCCACAGACTTCTCTCTG | |
| | | | CTTCAGCGAATCTATTCCGACGCCTTCCAATCG | |
| | | | CGAGGAAACTCAGCAAAAGTCCAATCTGGAAC | |
| | | | TACTCCGCATTTCTCTGCTTCTGATTCAGAGCTG | |
| | | | GCTAGAACCAGTGCAATTTCTGCGTTCCGTCTT | |
| | | | CGCCAATAGCCTAGTTTATGGCGCATCCGACAG | |
| | | | CAACGTATACGATCTCCTGAAAGATCTCGAGGA | |
| | | | AGGCATTCAGACCCTGATGGGTCGTCTCGAGGA | |
| | | | TGGCTCTCCGCGTACTGGTCAGATCTTCAAGCA | |
| | | | GACTTACTCTAAATTTGATACTAACAGCCACAA | |
| | | | TGACGATGCGCTTCTAAAAAACTATGGTCTGCT | |
| | | | GTATTGTTTTCGTAAAGATATGGACAAAGTTGA | |
| | | | AACCTTCCTGCGTATTGTTCAGTGTCGTTCCGTT | |
| | | | GAGGGCAGCTGTGGTTTCTAA | |
| AE912- | AEPAGSPTSTEEGTPGS | 5 | ATGGCTGAACCTGCTGGCTCTCCACCTCCACT | 11 |
| hGH- | GTASSSPGSSTPSGATG | | GAGGAAGGTACCCCGGGTAGCGGTACTGCTTCT | |
| AE288 | SPGASPGTSSTGSPGSP | | TCCTCTCCAGGTAGCTCTACCCCTTCTGGTGCAA | |
| | AGSPTSTEEGTSESATP | | CCGGCTCTCCAGGTGCTTCTCCGGGCACCAGCT | |
| | ESGPGTSTEPSEGSAPG | | CTACCGGTTCTCCAGGTAGCCCGGCTGGCTCTC | |
| | SPAGSPTSTEEGTSTEPS | | CTACCGGTTCTCCAGGTAGCCCGGCTGGCTCTC | |
| | EGSAPGTSTEPSEGSAP | | CTACTCCTGAGTCTGGTCCAGGTACCTCTACTG | |
| | GTSESATPESGPGSEPA | | AACCGTCCGAAGGTAGCGCTCCAGGTAGCCCA | |
| | TSGSETPGSEPATSGSET | | GCAGGCTCTCCGACTTCCACTGAGGAAGGTACT | |
| | PGSPAGSPTSTEEGTSES | | TCTACTGAACCTTCCGAAGGCAGCGCACCAGGT | |
| | ATPESGPGTSTEPSEGS | | ACCTCTACTGAACCTTCTGAGGGCAGCGCTCCA | |
| | APGTSTEPSEGSAPGSP | | GGTACTTCTGAAAGCGCTACCCCGGAATCTGGC | |
| | AGSPTSTEEGTSTEPSE | | CCAGGTAGCGAACCGGCTACTTCTGGTTCTGAA | |
| | GSAPGTSTEPSEGSAPG | | ACCCCAGGTAGCGAACCGGCTACCTCCGGTTCT | |
| | TSESATPESGPGTSTEPS | | GAAACTCCAGGTAGCCCGGCAGGCTCTCCGACC | |
| | EGSAPGTSESATPESGP | | TCTACTGAGGAAGGTACTTCTGAAAGCGCAACC | |
| | GSEPATSGSETPGTSTEP | | CCGGAGTCCGGCCCAGGTACCTCTACCGAACCG | |
| | SEGSAPGTSTEPSEGSA | | TCTGAGGGCAGCGCACCAGGTACTTCTACCGAA | |
| | PGTSESATPESGPGTSES | | CCGTCCGAGGGTAGCGCACCAGGTAGCCCAGC | |
| | ATPESGPGSPAGSPTST | | AGGTTCTCCTACCTCCACCGAGGAAGGTACTTC | |
| | EEGTSESATPESGPGSEP | | TACCGAACCGTCCGAGGGTAGCGCACCAGGTA | |
| | ATSGSETPGTSESATPES | | CCTCTACTGAACCTTCTGAGGGCAGCGCTCCAG | |
| | GPGTSTEPSEGSAPGTS | | GTACTTCTGAAAGCGCTACCCCGGAGTCCGGTC | |
| | TEPSEGSAPGTSTEPSEG | | CAGGTACTTCTACTGAACCGTCCGAAGGTAGCG | |
| | SAPGTSTEPSEGSAPGT | | CACAGGTACTTCTGAAAGCGCAACCCCTGAAT | |
| | STEPSEGSAPGTSTEPSE | | CCGGTCCAGGTAGCGAACCGGCTACTTCTGGCT | |
| | GSAPGSPAGSPTSTEEG | | CTGAGACTCCAGGTACTTCTACCGAACCGTCCG | |
| | TSTEPSEGSAPGTSESAT | | AAGGTAGCGCACCAGGTACTTCTACTGAACCGT | |
| | PESGPGSEPATSGSETP | | CTGAAGGTAGCGCACCAGGTACTTCTGAAAGCG | |
| | GTSESATPESGPGSEPA | | CAACCCCGGAATCCGGCCCAGGTACCTCTGAAA | |
| | TSGSETPGTSESATPESG | | GCGCAACCCCGGAGTCCGGCCCAGGTAGCCCTG | |
| | PGTSTEPSEGSAPGTSES | | CTGAAAGCGCAACCCCTGAATCCGGCCCAGGTA | |
| | ATPESGPGSPAGSPTST | | CTGAAAGCGCAACCCCTGAATCCGGCCCAGGTA | |
| | EEGSPAGSPTSTEEGSP | | GCGAACCGGCAACCTCCGGTTCTGAAACCCCAG | |
| | AGSPTSTEEGTSESATP | | QTACCTCTGAAAGCGCTACTCCGGAGTCTGGCC | |
| | ESGPGTSTEPSEGSAPG | | CAGGTACCTCTACTGAACCGTCTGAGGGTAGCG | |
| | TSESATPESGPGSEPATS | | CTCCAGGTACTTCTACTGAACCGTCCGAAGGTA | |
| | GSETPGTSESATPESGP | | GCGCACCAGGTACTTCTACCGAACCGTCCGAAG | |
| | GSEPATSGSETPGTSES | | GCAGCGCTCCAGGTACCTCTACTGAACCTTCCG | |
| | ATPESGPGTSTEPSEGS | | AGGGCAGCGCTCCAGGTACCTCTACCGAACCTT | |
| | APGSPAGSPTSTEEGTS | | CTGAAGGTAGCGCACCAGGTACTTCTACCGAAC | |
| | ESATPESGPGSEPATSG | | CGTCCGAGGGTAGCGCACCAGGTAGCCCAGCA | |
| | SETPGTSESATPESGPGS | | GGTTCTCCTACCTCCACCGAGGAAGGTACTTCT | |
| | PAGSPTSTEEGSPAGSP | | ACCGAACCGTCCGAGGGTAGCGCACCAGGTAC | |
| | TSTEEGTSTEPSEGSAP | | CTCTGAAAGCGCAACTCCTGAGTCTGGCCCAGG | |
| | GTSESATPESGPGTSES | | TAGCGAACCTGCTACCTCCGGCTCTGAGACTCC | |
| | ATPESGPGTSESATPES | | AGGTACCTCTGAAAGCGCAACCCCGGAATCTGG | |
| | GPGSEPATSGSETPGSE | | TCCAGGTAGCGAACCTGCAACCTCTGGCTCTGA | |
| | PATSGSETPGSPAGSPTS | | AACCCCAGGTACCTCTGAAAGCGCTACTCCTGA | |
| | TEEGTSTEPSEGSAPGT | | ATCTGGCCCAGGTACTTCTACTGAACCGTCCGA | |
| | STEPSEGSAPGSEPATS | | GGGCAGCGCACCAGGTACTTCTGAAAGCGCTAC | |
| | GSETPGTSESATPESGP | | TCCTGAGTCCGGCCCAGGTAGCCCGGCTGGCTC | |
| | GTSTEPSEGSAPGFPTIP | | TCCGACTTCCACCGAGGAAGGTAGCCCGGCTGG | |
| | LSRLFDNAMLRAHRLH | | CTCTCCAACTTCTACTGAAGAAGGTAGCCCGGC | |
| | QLAFDTYQEFEEAYIPK | | AGGCTCTCCGACCTCTACTGAGGAAGGTACTTC | |
| | EQKYSFLQNPQTSLCFS | | TGAAAGCGCAACCCCGGAGTCCGGCCCAGGTA | |
| | ESIPTPSNREETQQKSNL | | CCTCTACCGAACCGTCTGAGGGCAGCGCACCAG | |
| | ELLRISELLIQSWLEPVQ | | GTACCTCTGAAAGCGCAACTCCTGAGTCTGGCC | |
| | FLRSVFANSLVYGASDS | | CAGGTAGCGAACCTGCTACCTCCGGCTCTGAGA | |
| | NVYDLLKDLEEGIQTL | | CTCCAGGTACCTCTGAAAGCGCAACCCCGGAAT | |
| | MGRLEDGSPRTGQIFK | | CTGGTCCAGGTAGCGAACCTGCAACCTCTGGCT | |
| | QTYSKFDTNSHNDDAL | | CTGAAACCCCAGGTACCTCTGAAAGCGCTACTC | |
| | LKNYGLLYCFRKDMD | | CTGAATCTGGCCCAGGTACTTCTACTGAACCGT | |
| | KVETFLRIVQCRSVEGS | | CCGAGGGCAGCGCACCAGGTAGCCCTGCTGGCT | |
| | CGFGGTSESATPESGPG | | CTCCAACCTCCACCGAAGAAGGTACCTCTGAAA | |
| | SEPATSGSETPGTSESAT | | GCGCAACCCCTGAATCCGGCCCAGGTAGCGAA | |
| | PESGPGSEPATSGSETP | | CCGGCAACCTCCGGTTCTGAAACCCCAGGTACT | |
| | GTSESATPESGPGTSTEP | | TCTGAAAGCGCTACTCCTGAGTCCGGCCCAGGT | |
| | SEGSAPGSPAGSPTSTE | | AGCCCGGCTGGCTCTCCGACTTCCACCGAGGAA | |
| | EGTSESATPESGPGSEP | | GGTAGCCCGGCTGGCTCTCCAACTTCTACTGAA | |
| | ATSGSETPGTSESATPES | | GAAGGTACTTCTACCGAACCTTCCGAGGGCAGC | |
| | GPGSPAGSPTSTEEGSP | | GCACCAGGTACTTCTGAAAGCGCTACCCCTGAG | |
| | AGSPTSTEEGTSTEPSE | | TCCGGCCCAGGTACTTCTGAAAGCGCTACTCCT | |
| | GSAPGTSESATPESGPG | | GAATCCGGTCCAGGTACTTCTGAAAGCGCTACC | |
| | TSESATPESGPGTSESAT | | CCGGAATCTGGCCCAGGTAGCGAACCGGCTACT | |
| | PESGPGSEPATSGSETP | | TCTGGTTCTGAAACCCCAGGTAGCGAACCGGCT | |
| | GSEPATSGSETPGSPAG | | ACCTCCGGTTCTGAAACTCCAGGTAGCCCAGCA | |
| | SPTSTEEGTSTEPSEGSA | | GGCTCTCCGACTTCCACTGAGGAAGGTACTTCT | |
| | PGTSTEPSEGSAPGSEP | | ACTGAACCTTCCGAAGGCAGCGCACCAGGTACC | |
| | ATSGSETPGTSESATPES | | TCTACTGAACCTTCTGAGGGCAGCGCTCCAGGT | |
| | GPGTSTEPSEGSAPG | | AGCGAACCTGCAACCTCTGGCTCTGAAACCCCA | |
| | | | GGTACCTCTGAAAGCGCTACTCCTGAATCTGGC | |
| | | | CCAGGTACTTCTACTGAACCGTCCGAGGGCAGC | |
| | | | GCACCAGGTTTTCCGACTATTCCGCTGTCTCGTC | |
| | | | TGTTTGATAATGCTATGCTGCGTGCGCACCGTC | |
| | | | TGCACCAGCTGGCCTTTGATACTTACCAGGAAT | |
| | | | TTGAAGAAGCcTACATTCCTAAAGAGCAGAAGT | |
| | | | ACTCTTTCCTGCAAAACCCACAGACTTCTCTCTG | |
| | | | CTTCAGCGAATCTATTCCGACGCCTTCCAATCG | |
| | | | CGAGGAAACTCAGCAAAAGTCCAATCTGGAAC | |
| | | | TACTCCGCATTTCTCTGCTTCTGATTCAGAGCTG | |
| | | | GCTAGAACCAGTGCAATTTCTGCGTTCCGTCTT | |
| | | | CGCCAATAGCCTAGTTTATGGCGCATCCGACAG | |
| | | | CAACGTATACGATCTCCTGAAAGATCTCGAGGA | |
| | | | AGGCATTCAGACCCTGATGGGTCGTCTCGAGGA | |
| | | | TGGCTCTCCGCGTACTGGTCAGATCTTCAAGCA | |
| | | | GACTTACTCTAAATTTGATACTAACAGCCACAA | |
| | | | TGACGATGCGCTTCTAAAAAACTATGGTCTGCT | |
| | | | GTATTGTTTTCGTAAAGATATGGACAAAGTTGA | |
| | | | AACCTTCCTGCGTATTGTTCAGTGTCGTTCCGTT | |
| | | | GAGGGCAGCTGTGGTTTCTAAGGTGGTACCTCT | |
| | | | GAAAGCGCAACTCCTGAGTCTGGCCCAGGTAGC | |
| | | | GAACCTGCTACCTCCGGCTCTGAGACTCCAGGT | |
| | | | ACCTCTGAAAGCGCAACCCCGGAATCTGGTCCA | |
| | | | GGTAGCGAACCTGCAACCTCTGGCTCTGAAACC | |
| | | | CCAGGTACCTCTGAAAGCGCTACTCCTGAATCT | |
| | | | GGCCCAGGTACTTCTACTGAACCGTCCGAGGGC | |
| | | | AGCGCACCAGGTAGCCCTGCTGGCTCTCCAACC | |
| | | | TCCACCGAAGAAGGTACCTCTGAAAGCGCAAC | |
| | | | CCCTGAATCCGGCCCAGGTAGCGAACCGGCAA | |
| | | | CCTCCGGTTCTGAAACCCCAGGTACTTCTGAAA | |
| | | | GCGCTACTCCTGAGTCCGGCCCAGGTAGCCCGG | |
| | | | CTGGCTCTCCGACTTCCACCGAGGAAGGTAGCC | |
| | | | CGGCTGGCTCTCCAACTTCTACTGAAGAAGGTA | |
| | | | CTTCTACCGAACCTTCCGAGGGCAGCGCACCAG | |
| | | | GTACTTCTGAAAGCGCTACCCCTGAGTCCGGCC | |
| | | | CAGGTACTTCTGAAAGCGCTACTCCTGAATCCG | |
| | | | GTCCAGGTACTTCTGAAAGCGCTACCCCGGAAT | |
| | | | CTGGCCCAGGTAGCGAACCGGCTACTTCTGGTT | |
| | | | CTGAAACCCCAGGTAGCGAACCGGCTACCTCCG | |
| | | | GTTCTGAAACTCCAGGTAGCCCAGCAGGCTCTC | |
| | | | CGACTTCCACTGAGGAAGGTACTTCTACTGAAC | |
| | | | CTTCCGAAGGCAGCGCACCAGGTACCTCTACTG | |
| | | | AACCTTCTGAGGGCAGCGCTCCAGGTAGCGAAC | |
| | | | CTGCAACCTCTGGCTCTGAAACCCCAGGTACCT | |
| | | | CTGAAAGCGCTACTCCTGAATCTGGCCCAGGTA | |
| | | | CTTCTACTGAACCGTCCGAGGGCAGCGCACCA | |
| AM875- | GTSTEPSEGSAPGSEPA | 6 | GGTACTTCTACTGAACCGTCTGAAGGCAGCGCA | 12 |
| hGH | TSGSETPGSPAGSPTSTE | | CCAGGTAGCGAACCGGCTACTTCCGGTTCTGAA | |
| | EGSTSSTAESPGPGTSTP | | ACCCCAGGTAGCCCAGCAGGTTCTCCAACTTCT | |
| | ESGSASPGSTSESPSGTA | | ACTGAAGAAGGTTCTACCAGCTCTACCGCAGAA | |
| | PGSTSESPSGTAPGTSTP | | TCTCCTGGTCCAGGTACCTCTACTCCGGAAAGC | |
| | ESGSASPGTSTPESGSAS | | GGCTCTGCATCTCCAGGTTCTACTAGCGAATCT | |
| | PGSEPATSGSETPGTSES | | CCTTCTGGCACTGCACCAGGTTCTACTAGCGAA | |
| | ATPESGPGSPAGSPTST | | TCCCCGTCTGGTACTGCTCCAGGTACTTCTACTC | |
| | EEGTSTEPSEGSAPGTS | | CTGAAAGCGGTTCCGCTTCTCCAGGTACCTCTA | |
| | ESATPESGPGTSTEPSEG | | CTCCGGAAAGCGGTTCTGCATCTCCAGGTAGCG | |
| | SAPGTSTEPSEGSAPGSP | | AACCGGCAACCTCCGGCTCTGAAACCCCAGGTA | |
| | AGSPTSTEEGTSTEPSE | | CCTCTGAAAGCGCTACTCCTGAATCCGGCCCAG | |
| | GSAPGTSTEPSEGSAPG | | GTAGCCCGGCAGGTTCTCCGACTTCCACTGAGG | |
| | TSESATPESGPGTSESAT | | AAGGTACCTCTACTGAACCTTCTGAGGGCAGCG | |
| | PESGPGTSTEPSEGSAP | | CTCCAGGTACTTCTGAAAGCGCTACCCCGGAGT | |
| | GTSTEPSEGSAPGTSES | | CCGGTCCAGGTACTTCTACTGAACCGTCCGAAG | |
| | ATPESGPGTSTEPSEGS | | GTAGCGCACCAGGTACTTCTACCGAACCGTCCG | |
| | APGSEPATSGSETPGSP | | AGGGTAGCGCACCAGGTAGCCCAGCAGGTTCTC | |
| | AGSPTSTEEGSSTPSGA | | CTACCTCCACCGAGGAAGGTACTTCTACCGAAC | |
| | TGSPGTPGSGTASSSPG | | CGTCCGAGGGTAGCGCACCAGGTACTTCTACCG | |
| | SSTPSGATGSPGTSTEPS | | AACCTTCCGAGGGCAGCGCACCAGGTACTTCTG | |
| | EGSAPGTSTEPSEGSAP | | AAAGCGCTACCCCTGAGTCCGGCCCAGGTACTT | |
| | GSEPATSGSETPGSPAG | | CTGAAAGCGCTACTCCTGAATCCGGTCCAGGTA | |
| | SPTSTEEGSPAGSPTSTE | | CCTCTACTGAACCTTCCGAAGGCAGCGCTCCAG | |
| | EGTSTEPSEGSAPGASA | | GTACCTCTACCGAACCGTCCGAGGGCAGCGCAC | |
| | SGAPSTGGTSESATPES | | CAGGTACTTCTGAAAGCGCAACCCCTGAATCCG | |
| | GPGSPAGSPTSTEEGSP | | GTCCAGGTACTTCTACTGAACCTTCCGAAGGTA | |
| | AGSPTSTEEGSTSSTAES | | GCGCTCCAGGTAGCGAACCTGCTACTTCTGGTT | |
| | PGPGSTSESPSGTAPGTS | | CTGAAACCCCAGGTAGCCCGGCTGGCTCTCCGA | |
| | PSGESSTAPGTPGSGTA | | CCTCCACCGAGGAAGGTAGCTCTACCCCGTCTG | |
| | SSSPGSSTPSGATGSPGS | | GTGCTACTGGTTCTCCAGGTACTCCGGGCAGCG | |
| | SPSASTGTGPGSEPATS | | GTACTGCTTCTTCCTCTCCAGGTAGCTCTACCCC | |
| | GSETPGTSESATPESGP | | TTCTGGTGCTACTGGCTCTCCAGGTACCTCTACC | |
| | GSEPATSGSETPGSTSST | | GAACCGTCCGAGGGTAGCGCACCAGGTACCTCT | |
| | AESPGPGSTSSTAESPGP | | ACTGAACCGTCTGAGGGTAGCGCTCCAGGTAGC | |
| | GTSPSGESSTAPGSEPA | | GAACCGGCAACCTCCGGTTCTGAAACTCCAGGT | |
| | TSGSETPGSEPATSGSET | | AGCCCTGCTGGCTCTCCGACTTCTACTGAGGAA | |
| | PGTSTEPSEGSAPGSTSS | | GGTAGCCCGGCTGGTTCTCCGACTTCTACTGAG | |
| | TAESPGPGTSTPESGSA | | GAAGGTACTTCTACCGAACCTTCCGAAGGTAGC | |
| | SPGSTSESPSGTAPGTST | | GCTCCAGGTGCAAGCGCAAGCGGCGCGCCAAG | |
| | EPSEGSAPGTSTEPSEGS | | CACGGGAGGTACTTCTGAAAGCGCTACTCCTGA | |
| | APGTSTEPSEGSAPGSS | | GTCCGGCCCAGGTAGCCCGGCTGGCTCTCCGAC | |
| | TPSGATGSPGSSPSAST | | TTCCACCGAGGAAGGTAGCCCGGCTGGCTCTCC | |
| | GTGPGASPGTSSTGSPG | | AACTTCTACTGAAGAAGGTTCTACCAGCTCTAC | |
| | SEPATSGSETPGTSESAT | | CGCTGAATCTCCTGGCCCAGGTTCTACTAGCGA | |
| | PESGPGSPAGSPTSTEE | | ATCTCCGTCTGGCACCGCACCAGGTACTTCCCC | |
| | GSSTPSGATGSPGSSPS | | TAGCGGTGAATCTTCTACTGCACCAGGTACCCC | |
| | ASTGTGPGASPGTSSTG | | TGGCAGCGGTACCGCTTCTTCCTCTCCAGGTAG | |
| | SPGTSESATPESGPGTST | | CTCTACCCCGTCTGGTGCTACTGGCTCTCCAGGT | |
| | EPSEGSAPGTSTEPSEGS | | TCTAGCCCGTCTGCATCTACCGGTACCGGCCCA | |
| | APGFPTIPLSRLFDNAM | | GGTAGCGAACCGGCAACCTCCGGCTCTGAAACT | |
| | LRAHRLHQLAFDTYQE | | CCAGGTACTTCTGAAAGCGCTACTCCGGAATCC | |
| | FEEAYIPKEQKYSFLQN | | GGCCCAGGTAGCGAACCGGCTACTTCCGGCTCT | |
| | PQTSLCFSESIPTPSNRE | | GAAACCCCAGGTTCCACCAGCTCTACTGCAGAA | |
| | ETQQKSNLELLRISLLLI | | TCTCCGGGCCCAGGTTCTACTAGCTCTACTGCA | |
| | QSWLEPVQFLRSVFAN | | GAATCTCCGGGTCCAGGTACTTCTCCTAGCGGC | |
| | SLVYGASDSNVYDLLK | | GAATCTTCTACCGCTCCAGGTAGCGAACCGGCA | |
| | DLEEGIQTLMGRLEDGS | | ACCTCTGGCTCTGAAACTCCAGGTAGCGAACCT | |
| | PRTGQIFKQTYSKFDTN | | GCAACCTCCGGCTCTGAAACCCCAGGTACTTCT | |
| | SHNDDALLKNYGLLYC | | ACTGAACCTTCTGAGGGCAGCGCACCAGGTTCT | |
| | FRKDMDKVETFLRIVQ | | ACCAGCTCTACCGCAGAATCTCCTGGTCCAGGT | |
| | CRSVEGSCGF | | ACCTCTACTCCGGAAAGCGGCTCTGCATCTCCA | |
| | | | GGTTCTACTAGCGAATCTCCTTCTGGCACTGCA | |
| | | | CCAGGTACTTCTACCGAACCGTCCGAAGGCAGC | |
| | | | GCTCCAGGTACCTCTACTGAACCTTCCGAGGGC | |
| | | | AGCGCTCCAGGTACCTCTACCGAACCTTCTGAA | |
| | | | GGTAGCGCACCAGGTAGCTCTACTCCGTCTGGT | |
| | | | GCAACCGGCTCCCCAGGTTCTAGCCCGTCTGCT | |
| | | | TCCACTGGTACTGGCCCAGGTGCTTCCCCGGGC | |
| | | | ACCAGCTCTACTGGTTCTCCAGGTAGCGAACCT | |
| | | | GCTACCTCCGGTTCTGAAACCCCAGGTACCTCT | |
| | | | GAAAGCGCAACTCCGGAGTCTGGTCCAGGTAG | |
| | | | CCCTGCAGGTTCTCCTACCTCCACTGAGGAAGG | |
| | | | TAGCTCTACTCCGTCTGGTGCAACCGGCTCCCC | |
| | | | AGGTTCTAGCCCGTCTGCTTCCACTGGTACTGG | |
| | | | CCCAGGTGCTTCCCCGGGCACCAGCTCTACTGG | |
| | | | TTCTCCAGGTACCTCTGAAAGCGCTACTCCGGA | |
| | | | GTCTGGCCCAGGTACCTCTACTGAACCGTCTGA | |
| | | | GGGTAGCGCTCCAGGTACTTCTACTGAACCGTC | |
| | | | CGAAGGTAGCGCACCAGGTTTTCCGACTATTCC | |
| | | | GCTGTCTCGTCTGTTTGATAATGCTATGCTGCGT | |
| | | | GCGCACCGTCTGCACCAGCTGGCCTTTGATACT | |
| | | | TACCAGGAATTTGAAGAAGCcTACATTCCTAAA | |
| | | | GAGCAGAAGTACTCTTTCCTGCAAAACCCACAG | |
| | | | ACTTCTCTCTGCTTCAGCGAATCTATTCCGACGC | |
| | | | CTTCCAATCGCGAGGAAACTCAGCAAAAGTCCA | |
| | | | ATCTGGAACTACTCCGCATTTCTCTGCTTCTGAT | |
| | | | TCAGAGCTGGCTAGAACCAGTGCAATTTCTGCG | |
| | | | TTCCGTCTTCGCCAATAGCCTAGTTTATGGCGC | |
| | | | ATCCGACAGCAACGTATACGATCTCCTGAAAGA | |
| | | | TCTCGAGGAAGGCATTCAGACCCTGATGGGTCG | |
| | | | TCTCGAGGATGGCTCTCCGCGTACTGGTCAGAT | |
| | | | CTTCAAGCAGACTTACTCTAAATTTGATACTAA | |
| | | | CAGCCACAATGACGATGCGCTTCTAAAAAACTA | |
| | | | TGGTCTGCTGTATTGTTTTCGTAAAGATATGGA | |
| | | | CAAAGTTGAAACCTTCCTGCGTATTGTTCAGTG | |
| | | | TCGTTCCGTTGAGGGCAGCTGTGGTTTCTAA | |

Further characterization of the exemplary hGH-XTEN fusion proteins provided in Table 1 can be found in the examples (e.g., Examples 27-35) of Schellenberger et al. WO10/144502A2, which is incorporated herein by reference in its entirety.

The present invention contemplates use of hGH-XTEN fusion proteins comprising one of the amino acid sequences shown in FIG. 1, Table 1, or as described in Schellenberger et al. WO10/144502A2 (which is incorporated herein by reference in its entirety). In addition, pharmacologically active variants of any of the hGH-XTEN fusion proteins described and referred to herein are also contemplated.

As described more fully below, the fusion proteins optionally include spacer sequences that further comprise cleavage sequences to release the GH from the fusion protein when acted on by a protease, releasing GH from the XTEN sequence(s).

In one aspect, the invention provides an isolated fusion protein comprising at least a first biologically active growth hormone protein covalently linked to one or more extended recombinant polypeptides (“XTEN”), resulting in a growth hormone-XTEN fusion protein composition (hereinafter “hGH-XTEN”). In one embodiment, the growth hormone is human growth hormone or a sequence variant of hGH. As described more fully below, the fusion proteins optionally include spacer sequences that further comprise cleavage sequences to release the GH from the fusion protein when acted on by a protease.

The term “hGH-XTEN”, as used herein, is meant to encompass fusion polypeptides that comprise a payload region comprising a biologically active GH that mediates one or more biological or therapeutic activities associated with growth hormone and at least one other region comprising at least a first XTEN polypeptide that serves as a carrier. In one embodiment, the invention provides an hGH-XTEN fusion protein comprising the sequence set forth in Table 1.

The GH of the subject compositions, together with their corresponding nucleic acid and amino acid sequences, are well known in the art and descriptions and sequences are available in public databases such as Chemical Abstracts Services Databases (e.g., the CAS Registry), GenBank, The Universal Protein Resource (UniProt) and subscription provided databases such as GenSeq (e.g., Derwent). Polynucleotide sequences may be a wild type polynucleotide sequence encoding a given GH (e.g., either full length or mature), or in some instances the sequence may be a variant of the wild type polynucleotide sequence (e.g., a polynucleotide which encodes the wild type biologically active protein, wherein the DNA sequence of the polynucleotide has been optimized, for example, for expression in a particular species; or a polynucleotide encoding a variant of the wild type protein, such as a site directed mutant or an allelic variant. It is well within the ability of the skilled artisan to use a wild-type or consensus cDNA sequence or a codon-optimized variant of a GH to create fusion protein constructs contemplated by the invention using methods known in the art and/or in conjunction with the guidance and methods provided herein, and described more fully in the Examples of Schellenberger et al. WO10/144502A2 which is incorporated herein by reference in its entirety.

The GH for inclusion in the hGH-XTEN of the invention include any growth hormone or sequence variant of biologic, therapeutic, prophylactic, or diagnostic interest or function, or that is useful for mediating or preventing or ameliorating a disease, disorder or condition associated with growth, growth hormone deficiency or defect when administered to a pediatric subject. Of particular interest are hGH-XTEN fusion protein compositions for which an increase in a pharmacokinetic parameter, increased solubility, increased stability, or some other enhanced pharmaceutical or pharmacodynamic property compared to native GH is sought, or for which increasing the terminal half-life would improve efficacy, safety, or result in reduce dosing frequency and/or improve pediatric patient compliance. Thus, the hGH-XTEN fusion protein compositions are prepared with various objectives in mind, including improving the therapeutic efficacy of the bioactive GH by, for example, increasing the in vivo exposure or the length that the hGH-XTEN remains within the therapeutic window when administered to a pediatric subject, compared to a GH not linked to XTEN.

In one embodiment, the GH incorporated into the subject compositions can be a recombinant polypeptide with a sequence corresponding to a protein found in nature, such as human growth hormone. In one embodiment, the GH is human GH comprising the following amino acid sequence: FPTIPLSRLFDNAMLRAHRLHQLAFDTYQEFEEAYIPKEQKYSFLQNPQTSLCFSES IPTPSNREETQQKSNLELLRISLLLIQSWLEPVQFLRSVFANSLVYGASDSNVYDLL KDLEEGIQTLMGRLEDGSPRTGQIFKQTYSKFDTNSHNDDALLKNYGLLYCFRKD MDKVETFLRIVQCRSVEGSCGF (SEQ ID NO:41).

In another embodiment, the GH is a sequence variant, fragment, homolog, or mimetic of a natural sequence that retain at least a portion of the biological activity of the native GH. In non-limiting examples, a GH is a sequence that exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity to the protein sequence of SEQ ID NO:41. In one embodiment, the hGH-XTEN fusion protein comprises a single GH molecule linked to an XTEN (as described more fully below). In another embodiment, the hGH-XTEN fusion protein comprises a single GH molecule linked to a first and a second XTEN, with an N- to C-terminus configuration of XTEN-GH-XTEN, in which the GH is a sequence that exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity to the human growth hormone protein sequence (SEQ ID NO:41), and the first and/or the second XTEN are sequences that exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, or at least about 99%, or 100% sequence identity to a sequence selected from Table 2.

In general, the GH fusion partner component of the hGH-XTEN exhibits a binding specificity to a given target or another desired biological characteristic when used in vivo or when utilized in an in vitro assay. For example, the hGH-XTEN is an agonist, having the ability to bind to a transmembrane receptor for growth hormone. In one embodiment, the binding of hGH-XTEN to growth receptor leads to receptor dimerization and lead to at least a portion of the activation of intercellular signal transduction pathway compared to native growth hormone. In one embodiment, the hGH-XTEN bound to a transmembrane receptor for growth hormone would exhibit at least about 1%, or about 5%, or about 10%, or about 15%, or about 20%, or about 25%, or about 30%, or about 40%, or about 50%, or about 60%, or about 70%, or about 80%, or about 90%, or at least about 95% of the activation of intercellular signal transduction pathway compared to native growth hormone not linked to XTEN.

The subject hGH-XTEN of the present invention exhibits an enhancement of one or more pharmacokinetic or pharmacodynamic parameters, which optionally is enhanced by release of GH from the fusion protein by cleavage of a spacer sequence. The hGH-XTEN with enhanced pharmacokinetic parameters permits less frequent dosing or an enhanced pharmacologic effect, such as but not limited to maintaining the biologically active hGH-XTEN within the therapeutic window between the minimum effective dose or blood concentration (Cmin) and the maximum tolerated dose or blood concentration (Cmax). In addition, the hGH-XTEN with enhanced pharmacodynamic parameters permits lower and/or less frequent dosing or an enhanced pharmacodynamic effect, such as but not limited to a sustained or normalized IGF-I standard deviation score (IGF-I SDS). In such cases, the linking of the GH to a fusion protein comprising a select XTEN sequence(s) can result in an improvement in these properties, making them more useful as therapeutic or preventive agents compared to GH not linked to XTEN.

### IV). Xtended Recombinant Polypeptides

The present invention concerns an improved therapeutic regimen for PGHD therapy. In particular, the invention concerns methods for bolus dose administration of a human growth hormone-XTEN (hGH-XTEN) fusion protein to a pediatric patient with PGHD. Accordingly, in one aspect, the present invention concerns a method of treating human pediatric growth hormone deficiency (PGHD) with a hGH-XTEN recombinant polypeptide or fusion protein.

In another aspect, the present invention provides XTEN polypeptide compositions that are useful as a fusion protein partner to which GH is linked, resulting in a hGH-XTEN fusion protein. XTEN are generally extended length polypeptides with non-naturally occurring, substantially non-repetitive sequences that are composed mainly of small hydrophilic amino acids, with the sequence having a low degree or no secondary or tertiary structure under physiologic conditions.

XTENs have utility as a fusion protein partners partner in that they serve as a “carrier”, conferring certain desirable pharmacokinetic, physicochemical and pharmaceutical properties when linked to a GH protein to a create a fusion protein. Such desirable properties include but are not limited to enhanced pharmacokinetic parameters and solubility characteristics the compositions, amongst other properties described herein. Such fusion protein compositions have utility to treat certain growth hormone-related diseases, disorders or conditions, as described herein. As used herein, “XTEN” specifically excludes antibodies or antibody fragments such as single-chain antibodies or Fc fragments of a light chain or a heavy chain.

In some embodiments, XTEN are long polypeptides having greater than about 100 to about 3000 amino acid residues, preferably greater than 400 to about 3000 residues when used as a carrier or cumulatively when more than one XTEN unit is used in a single fusion protein. In other embodiments, when used as a linker between fusion protein components or where an increase in half-life of the fusion protein is not needed but where an increase in solubility or other physico/chemical property for the GH fusion partner component is desired, an XTEN sequence shorter than 100 amino acid residues, such as about 96, or about 84, or about 72, or about 60, or about 48, or about 36 amino acid residues are incorporated into a fusion protein composition with the GH to effect the property.

The selection criteria for the XTEN to be linked to the biologically active proteins used to create the inventive fusion proteins compositions generally relate to attributes of physical/chemical properties and conformational structure of the XTEN that is, in turn, used to confer enhanced pharmaceutical and pharmacokinetic properties to the fusion proteins. The XTEN of the present invention exhibit one or more of the following advantageous properties: conformational flexibility, enhanced aqueous solubility, high degree of protease resistance, low immunogenicity, low binding to mammalian receptors, and increased hydrodynamic (or Stokes) radii; properties that make them particularly useful as fusion protein partners. Non-limiting examples of the properties of the fusion proteins comprising GH that is enhanced by XTEN include increases in the overall solubility and/or metabolic stability, reduced susceptibility to proteolysis, reduced immunogenicity, reduced rate of absorption when administered subcutaneously or intramuscularly, and enhanced pharmacokinetic properties such as longer terminal half-life and increased area under the curve (AUC), slower absorption after subcutaneous or intramuscular injection (compared to GH not linked to XTEN and administered by a similar route) such that the Cmax is lower, which, in turn, results in reductions in adverse effects of the GH that, collectively, results in an increased period of time that a fusion protein of a hGH-XTEN composition administered to a pediatric patient retains therapeutic activity.

1. Non-Repetitive Sequences

In some embodiments, XTEN sequences of the compositions are substantially non-repetitive. In general, repetitive amino acid sequences have a tendency to aggregate or form higher order structures, as exemplified by natural repetitive sequences such as collagens and leucine zippers, or form contacts resulting in crystalline or pseudocrystaline structures. In contrast, the low tendency of non-repetitive sequences to aggregate enables the design of long-sequence XTENs with a relatively low frequency of charged amino acids that would be likely to aggregate if the sequences were otherwise repetitive. Typically, the hGH-XTEN fusion proteins comprise XTEN sequences of greater than about 100 to about 3000 amino acid residues, preferably greater than 400 to about 3000 cumulative residues, wherein the sequences are substantially non-repetitive. In one embodiment, the XTEN sequences have greater than about 100 to about 3000 amino acid residues, preferably greater than 400 to about 3000 amino acid residues, in which no three contiguous amino acids in the sequence are identical amino acid types unless the amino acid is serine, in which case no more than three contiguous amino acids are serine residues. In the foregoing embodiment, the XTEN sequence would be substantially non-repetitive.

The degree of repetitiveness of a polypeptide or a gene are measured by computer programs or algorithms or by other means known in the art, including subsequence scores (see Example 44 of Schellenberger et al. WO10/144502A2; Cleland et al. U.S. Ser. No. 13/829,369; and Cleland et al. WO13/184216, each of which is incorporated herein by reference in its entirety). In some embodiments, the present invention provides hGH-XTEN each comprising one or more XTEN in which the XTEN have a subsequence score less than 12, more preferably less than 10, more preferably less than 9, more preferably less than 8, more preferably less than 7, more preferably less than 6, and most preferably less than 5. In the embodiments hereinabove described in this paragraph, an XTEN with a subsequence score less than about 10 (i.e., 9, 8, 7, etc.) is “substantially non-repetitive.”

The non-repetitive characteristic of XTEN impart to fusion proteins with GH a greater degree of solubility and less tendency to aggregate compared to polypeptides having repetitive sequences. These properties facilitate the formulation of XTEN-comprising pharmaceutical preparations containing extremely high drug concentrations, in some cases exceeding 100 mg/ml.

2. Exemplary Sequence Motifs

The present invention encompasses XTEN that comprise multiple units of shorter sequences, or motifs, in which the amino acid sequences of the motifs are non-repetitive. In designing XTEN sequences, it was discovered that the non-repetitive criterion may be met despite the use of a “building block” approach using a library of sequence motifs that are multimerized to create the XTEN sequences. Thus, while an XTEN sequence may consist of multiple units of as few as four different types of sequence motifs, because the motifs themselves generally consist of non-repetitive amino acid sequences, the overall XTEN sequence is rendered substantially non-repetitive (see Schellenberger et al. WO10/144502A2; Cleland et al. U.S. Ser. No. 13/829,369; and Cleland et al. WO13/184216, each of which is incorporated herein by reference in its entirety).

3. Length of Sequence

In another aspect of the present invention, the invention encompasses hGH-XTEN compositions comprising carriers of XTEN polypeptides with extended length sequences. (see Schellenberger et al. WO10/144502A2; Cleland et al. U.S. Ser. No. 13/829,369; and Cleland et al. PCT/US2013/031673, each of which is incorporated herein by reference in its entirety) Non-limiting examples of XTEN contemplated for inclusion in the hGH-XTEN of the invention are presented in Table 2. In one embodiment, the invention provides hGH-XTEN compositions wherein the XTEN sequence length of the fusion protein(s) is greater than about 100 to about 3000 amino acid residues, and in some cases is greater than 400 to about 3000 amino acid residues, wherein the XTEN confers enhanced pharmacokinetic properties on the hGH-XTEN in comparison to GH not linked to XTEN. In some embodiments, the XTEN sequences of the hGH-XTEN compositions of the present invention can be about 100, or about 144, or about 288, or about 401, or about 500, or about 600, or about 700, or about 800, or about 900, or about 1000, or about 1500, or about 2000, or about 2500 or up to about 3000 amino acid residues in length. In other cases, the XTEN sequences can be about 100 to 150, about 150 to 250, about 250 to 400, 401 to about 500, about 500 to 900, about 900 to 1500, about 1500 to 2000, or about 2000 to about 3000 amino acid residues in length. In one embodiment, the hGH-XTEN can comprise an XTEN sequence wherein the sequence exhibits at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity to a XTEN selected from Table 2. In some embodiments, the XTEN sequence is designed for optimized expression as the N-terminal component of the hGH-XTEN by inclusion of encoding nucleotides for an optimized N-terminal leader sequence (NTS) in the XTEN portion of the gene encoding the fusion protein. In another embodiment, the N-terminal XTEN sequence of the expressed hGH-XTEN has at least 90% sequence identity to any sequence selected from Table 2. In one embodiment, the N-terminal XTEN sequence of the expressed hGH-XTEN has at least 90% sequence identity to the sequence of AE48 or AM48, AE624, AE911, AE912 or AM923.

In other embodiments, the hGH-XTEN fusion protein comprises a first and a second XTEN sequence, wherein the cumulative total of the residues in the XTEN sequences is greater than about 400 to about 3000 amino acid residues. In embodiments of the foregoing, the hGH-XTEN fusion protein comprises a first and a second XTEN sequence wherein the sequences each exhibit at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity to at least a first or additionally a second XTEN selected from Table 2. Examples where more than one XTEN is used in a hGH-XTEN composition include, but are not limited to constructs with an XTEN linked to both the N- and C-termini of at least one GH.

As described more fully below, the invention provides methods in which the hGH-XTEN is designed by selecting the length of the XTEN to confer a target half-life on a fusion protein administered to a pediatric subject. In general, XTEN lengths longer that about cumulative 400 residues incorporated into the hGH-XTEN compositions result in longer half-life compared to shorter cumulative lengths; e.g., shorter than about 280 residues. However, in another embodiment, hGH-XTEN fusion proteins are designed to comprise XTEN with a longer sequence length that is selected to additionally confer slower rates of systemic absorption after subcutaneous or intramuscular administration to a pediatric subject. In such embodiments, the Cmax is reduced in comparison to a comparable dose of a GH not linked to XTEN, thereby contributing to the ability to keep the hGH-XTEN within the therapeutic window for the composition. Thus, the XTEN confers the property of a depot to the administered hGH-XTEN, in addition to the other physical/chemical properties described herein.

TABLE 2
XTEN Polypeptides

| | SEQ | |
|---|---|---|
| XTEN | ID | |
| Name | NO: | Amino Acid Sequence |
| AE48 | 13 | MAEPAGSPTSTEEGTPGSGTASSSPGSSTPSGATGSPGASPGTSSTGS |
| AM48 | 14 | MAEPAGSPTSTEEGASPGTSSTGSPGSSTPSGATGSPGSSTPSGATGS |
| AE144 | 15 | GSEPATSGSETPGTSESATPESGPGSEPATSGSETPGSPAGSPTSTEEGTSTEPSEGS |
| | | APGSEPATSGSETPGSEPATSGSETPGSEPATSGSETPGTSTEPSEGSAPGTSESATP |
| | | ESGPGSEPATSGSETPGTSTEPSEGSAP |
| AF144 | 16 | GTSTPESGSASPGTSPSGESSTAPGTSPSGESSTAPGSTSSTAESPGPGSTSESPSGTA |
| | | PGSTSSTAESPGPGTSPSGESSTAPGTSTPESGSASPGSTSSTAESPGPGTSPSGESST |
| | | APGTSPSGESSTAPGTSPSGESSTAP |
| AE288 | 17 | GTSESATPESGPGSEPATSGSETPGTSESATPESGPGSEPATSGSETPGTSESATPES |
| | | GPGTSTEPSEGSAPGSPAGSPTSTEEGTSESATPESGPGSEPATSGSETPGTSESATP |
| | | ESGPGSPAGSPTSTEEGSPAGSPTSTEEGTSTEPSEGSAPGTSESATPESGPGTSESA |
| | | TPESGPGTSESATPESGPGSEPATSGSETPGSEPATSGSETPGSPAGSPTSTEEGTST |
| | | EPSEGSAPGTSTEPSEGSAPGSEPATSGSETPGTSESATPESGPGTSTEPSEGSAP |
| AF504 | 18 | GASPGTSSTGSPGSSPSASTGTGPGSSPSASTGTGPGTPGSGTASSSPGSSTPSGATG |
| | | SPGSXPSASTGTGPGASPGTSSTGSPGTPGSGTASSSPGSSTPSGATGSPGTPGSGT |
| | | ASSSPGASPGTSSTGSPGASPGTSSTGSPGTPGSGTASSSPGSSTPSGATGSPGASPG |
| | | TSSTGSPGTPGSGTASSSPGSSTPSGATGSPGSXPSASTGTGPGSSPSASTGTGPGSS |
| | | TPSGATGSPGSSTPSGATGSPGASPGTSSTGSPGASPGTSSTGSPGASPGTSSTGSPG |
| | | TPGSGTASSSPGASPGTSSTGSPGASPGTSSTGSPGASPGTSSTGSPGSSPSASTGTG |
| | | PGTPGSGTASSSPGASPGTSSTGSPGASPGTSSTGSPGASPGTSSTGSPGSSTPSGAT |
| | | GSPGSSTPSGATGSPGASPGTSSTGSPGTPGSGTASSSPGSSTPSGATGSPGSSTPSG |
| | | ATGSPGSSTPSGATGSPGSSPSASTGTGPGASPGTSSTGSP |
| AF540 | 19 | GSTSSTAESPGPGSTSSTAESPGPGSTSESPSGTAPGSTSSTAESPGPGSTSSTAESPG |
| | | PGTSTPESGSASPGSTSESPSGTAPGTSPSGESSTAPGSTSESPSGTAPGSTSESPSGT |
| | | APGTSPSGESSTAPGSTSESPSGTAPGSTSESPSGTAPGTSPSGESSTAPGSTSESPSG |
| | | TAPGSTSESPSGTAPGSTSESPSGTAPGTSTPESGSASPGSTSESPSGTAPGTSTPESG |
| | | SASPGSTSSTAESPGPGSTSSTAESPGPGTSTPESGSASPGTSTPESGSASPGSTSESP |
| | | SGTAPGTSTPESGSASPGTSTPESGSASPGSTSESPSGTAPGSTSESPSGTAPGSTSES |
| | | PSGTAPGSTSSTAESPGPGTSTPESGSASPGTSTPESGSASPGSTSESPSGTAPGSTSE |
| | | SPSGTAPGTSTPESGSASPGSTSESPSGTAPGSTSESPSGTAPGTSTPESGSASPGTSP |
| | | SGESSTAPGSTSSTAESPGPGTSPSGESSTAPGSTSSTAESPGPGTSTPESGSASPGST |
| | | SESPSGTAP |
| AD576 | 20 | GSSESGSSEGGPGSGGEPSESGSSGSSESGSSEGGPGSSESGSSEGGPGSSESGSSEG |
| | | GPGSSESGSSEGGPGSSESGSSEGGPGESPGGSSGSESGSEGSSGPGESSGSSESGSS |
| | | EGGPGSSESGSSEGGPGSSESGSSEGGPGSGGEPSESGSSGESPGGSSGSESGESPG |
| | | GSSGSESGSGGEPSESGSSGSSESGSSEGGPGSGGEPSESGSSGSGGEPSESGSSGSE |
| | | GSSGPGESSGESPGGSSGSESGSGGEPSESGSSGSGGEPSESGSSGSGGEPSESGSSG |
| | | SSESGSSEGGPGESPGGSSGSESGESPGGSSGSESGESPGGSSGSESGESPGGSSGSE |
| | | SGESPGGSSGSESGSSESGSSEGGPGSGGEPSESGSSGSEGSSGPGESSGSSESGSSE |
| | | GGPGSGGEPSESGSSGSSESGSSEGGPGSGGEPSESGSSGESPGGSSGSESGESPGG |
| | | SSGSESGSSESGSSEGGPGSGGEPSESGSSGSSESGSSEGGPGSGGEPSESGSSGSGG |
| | | EPSESGSSGESPGGSSGSESGSEGSSGPGESSGSSESGSSEGGPGSEGSSGPGESS |
| AE576 | 21 | GSPAGSPTSTEEGTSESATPESGPGTSTEPSEGSAPGSPAGSPTSTEEGTSTEPSEGS |
| | | APGTSTEPSEGSAPGTSESATPESGPGSEPATSGSETPGSEPATSGSETPGSPAGSPT |
| | | STEEGTSESATPESGPGTSTEPSEGSAPGTSTEPSEGSAPGSPAGSPTSTEEGTSTEPS |
| | | EGSAPGTSTEPSEGSAPGTSESATPESGPGTSTEPSEGSAPGTSESATPESGPGSEPA |
| | | TSGSETPGTSTEPSEGSAPGTSTEPSEGSAPGTSESATPESGPGTSESATPESGPGSP |
| | | AGSPTSTEEGTSESATPESGPGSEPATSGSETPGTSESATPESGPGTSTEPSEGSAPG |
| | | TSTEPSEGSAPGTSTEPSEGSAPGTSTEPSEGSAPGTSTEPSEGSAPGTSTEPSEGSA |
| | | PGSPAGSPTSTEEGTSTEPSEGSAPGTSESATPESGPGSEPATSGSETPGTSESATPE |
| | | SGPGSEPATSGSETPGTSESATPESGPGTSTEPSEGSAPGTSESATPESGPGSPAGSP |
| | | TSTEEGSPAGSPTSTEEGSPAGSPTSTEEGTSESATPESGPGTSTEPSEGSAP |
| AF576 | 22 | GSTSSTAESPGPGSTSSTAESPGPGSTSESPSGTAPGSTSSTAESPGPGSTSSTAESPG |
| | | PGTSTPESGSASPGSTSESPSGTAPGTSPSGESSTAPGSTSESPSGTAPGSTSESPSGT |
| | | APGTSPSGESSTAPGSTSESPSGTAPGSTSESPSGTAPGTSPSGESSTAPGSTSESPSG |
| | | TAPGSTSESPSGTAPGSTSESPSGTAPGTSTPESGSASPGSTSESPSGTAPGTSTPESG |
| | | SASPGSTSSTAESPGPGSTSSTAESPGPGTSTPESGSASPGTSTPESGSASPGSTSESP |
| | | SGTAPGTSTPESGSASPGTSTPESGSASPGSTSESPSGTAPGSTSESPSGTAPGSTSES |
| | | PSGTAPGSTSSTAESPGPGTSTPESGSASPGTSTPESGSASPGSTSESPSGTAPGSTSE |
| | | SPSGTAPGTSTPESGSASPGSTSESPSGTAPGSTSESPSGTAPGTSTPESGSASPGTSP |
| | | SGESSTAPGSTSSTAESPGPGTSPSGESSTAPGSTSSTAESPGPGTSTPESGSASPGST |
| | | SESPSGTAPGSTSSTAESPGPGTSTPESGSASPGTSTPESGSASP |
| AE624 | 23 | MAEPAGSPTSTEEGTPGSGTASSSPGSSTPSGATGSPGASPGTSSTGSPGSPAGSPT |
| | | STEEGTSESATPESGPGTSTEPSEGSAPGSPAGSPTSTEEGTSTEPSEGSAPGTSTEPS |
| | | EGSAPGTSESATPESGPGSEPATSGSETPGSEPATSGSETPGSPAGSPTSTEEGTSES |
| | | ATPESGPGTSTEPSEGSAPGTSTEPSEGSAPGSPAGSPTSTEEGTSTEPSEGSAPGTS |
| | | TEPSEGSAPGTSESATPESGPGTSTEPSEGSAPGTSESATPESGPGSEPATSGSETPG |
| | | TSTEPSEGSAPGTSTEPSEGSAPGTSESATPESGPGTSESATPESGPGSPAGSPTSTE |
| | | EGTSESATPESGPGSEPATSGSETPGTSESATPESGPGTSTEPSEGSAPGTSTEPSEG |
| | | SAPGTSTEPSEGSAPGTSTEPSEGSAPGTSTEPSEGSAPGTSTEPSEGSAPGSPAGSP |
| | | TSTEEGTSTEPSEGSAPGTSESATPESGPGSEPATSGSETPGTSESATPESGPGSEPA |
| | | TSGSETPGTSESATPESGPGTSTEPSEGSAPGTSESATPESGPGSPAGSPTSTEEGSP |
| | | AGSPTSTEEGSPAGSPTSTEEGTSESATPESGPGTSTEPSEGSAP |
| AD836 | 24 | GSSESGSSEGGPGSSESGSSEGGPGESPGGSSGSESGSGGEPSESGSSGESPGGSSGS |
| | | ESGESPGGSSGSESGSSESGSSEGGPGSSESGSSEGGPGSSESGSSEGGPGESPGGSS |
| | | GSESGESPGGSSGSESGESPGGSSGSESGSSESGSSEGGPGSSESGSSEGGPGSSESG |
| | | SSEGGPGSSESGSSEGGPGSSESGSSEGGPGSSESGSSEGGPGSGGEPSESGSSGESP |
| | | GGSSGSESGESPGGSSGSESGSGGEPSESGSSGSEGSSGPGESSGSSESGSSEGGPGS |
| | | GGEPSESGSSGSEGSSGPGESSGSSESGSSEGGPGSGGEPSESGSSGESPGGSSGSES |
| | | GSGGEPSESGSSGSGGEPSESGSSGSSESGSSEGGPGSGGEPSESGSSGSGGEPSESG |
| | | SSGSEGSSGPGESSGESPGGSSGSESGSEGSSGPGESSGSEGSSGPGESSGSGGEPSE |
| | | SGSSGSSESGSSEGGPGSSESGSSEGGPGESPGGSSGSESGSGGEPSESGSSGSEGSS |
| | | GPGESSGESPGGSSGSESGSEGSSGPGSSESGSSEGGPGSGGEPSESGSSGSEGSSGP |
| | | GESSGSEGSSGPGESSGSEGSSGPGESSGSGGEPSESGSSGSGGEPSESGSSGESPGG |
| | | SSGSESGESPGGSSGSESGSGGEPSESGSSGSEGSSGPGESSGESPGGSSGSESGSSE |
| | | SGSSEGGPGSSESGSSEGGPGSSESGSSEGGPGSGGEPSESGSSGSSESGSSEGGPGE |
| | | SPGGSSGSESGSGGEPSESGSSGSSESGSSEGGPGESPGGSSGSESGSGGEPSESGSS |
| | | GESPGGSSGSESGSGGEPSESGSS |
| AE864 | 25 | GSPAGSPTSTEEGTSESATPESGPGTSTEPSEGSAPGSPAGSPTSTEEGTSTEPSEGS |
| | | APGTSTEPSEGSAPGTSESATPESGPGSEPATSGSETPGSEPATSGSETPGSPAGSPT |
| | | STEEGTSESATPESGPGTSTEPSEGSAPGTSTEPSEGSAPGSPAGSPTSTEEGTSTEPS |
| | | EGSAPGTSTEPSEGSAPGTSESATPESGPGTSTEPSEGSAPGTSESATPESGPGSEPA |
| | | TSGSETPGTSTEPSEGSAPGTSTEPSEGSAPGTSESATPESGPGTSESATPESGPGSP |
| | | AGSPTSTEEGTSESATPESGPGSEPATSGSETPGTSESATPESGPGTSTEPSEGSAPG |
| | | TSTEPSEGSAPGTSTEPSEGSAPGTSTEPSEGSAPGTSTEPSEGSAPGTSTEPSEGSA |
| | | PGSPAGSPTSTEEGTSTEPSEGSAPGTSESATPESGPGSEPATSGSETPGTSESATPE |
| | | SGPGSEPATSGSETPGTSESATPESGPGTSTEPSEGSAPGTSESATPESGPGSPAGSP |
| | | TSTEEGSPAGSPTSTEEGSPAGSPTSTEEGTSESATPESGPGTSTEPSEGSAPGTSES |
| | | ATPESGPGSEPATSGSETPGTSESATPESGPGSEPATSGSETPGTSESATPESGPGTS |
| | | TEPSEGSAPGSPAGSPTSTEEGTSESATPESGPGSEPATSGSETPGTSESATPESGPG |
| | | SPAGSPTSTEEGSPAGSPTSTEEGTSTEPSEGSAPGTSESATPESGPGTSESATPESG |
| | | PGTSESATPESGPGSEPATSGSETPGSEPATSGSETPGSPAGSPTSTEEGTSTEPSEG |
| | | SAPGTSTEPSEGSAPGSEPATSGSETPGTSESATPESGPGTSTEPSEGSAP |
| AF864 | 26 | GSTSESPSGTAPGTSPSGESSTAPGSTSESPSGTAPGSTSESPSGTAPGTSTPESGSAS |
| | | PGTSTPESGSASPGSTSESPSGTAPGSTSESPSGTAPGTSPSGESSTAPGSTSESPSGT |
| | | APGTSPSGESSTAPGTSPSGESSTAPGSTSSTAESPGPGTSPSGESSTAPGTSPSGESS |
| | | TAPGSTSSTAESPGPGTSTPESGSASPGTSTPESGSASPGSTSESPSGTAPGSTSESPS |
| | | GTAPGTSTPESGSASPGSTSSTAESPGPGTSTPESGSASPGSTSESPSGTAPGTSPSG |
| | | ESSTAPGSTSSTAESPGPGTSPSGESSTAPGTSTPESGSASPGSTSSTAESPGPGSTSS |
| | | TAESPGPGSTSSTAESPGPGSTSSTAESPGPGTSPSGESSTAPGSTSESPSGTAPGSTS |
| | | ESPSGTAPGTSTPESGPXXXGASASGAPSTXXXXSESPSGTAPGSTSESPSGTAPGS |
| | | TSESPSGTAPGSTSESPSGTAPGSTSESPSGTAPGSTSESPSGTAPGTSTPESGSASPG |
| | | TSPSGESSTAPGTSPSGESSTAPGSTSSTAESPGPGTSPSGESSTAPGTSTPESGSASP |
| | | GSTSESPSGTAPGSTSESPSGTAPGTSPSGESSTAPGSTSESPSGTAPGTSTPESGSAS |
| | | PGTSTPESGSASPGSTSESPSGTAPGTSTPESGSASPGSTSSTAESPGPGSTSESPSGT |
| | | APGSTSESPSGTAPGTSPSGESSTAPGSTSSTAESPGPGTSPSGESSTAPGTSTPESGS |
| | | ASPGTSPSGESSTAPGTSPSGESSTAPGTSPSGESSTAPGSTSSTAESPGPGSTSSTAE |
| | | SPGPGTSPSGESSTAPGSSPSASTGTGPGSSTPSGATGSPGSSTPSGATGSP |
| AG864 | 27 | GASPGTSSTGSPGSSPSASTGTGPGSSPSASTGTGPGTPGSGTASSSPGSSTPSGATG |
| | | SPGSSPSASTGTGPGASPGTSSTGSPGTPGSGTASSSPGSSTPSGATGSPGTPGSGTA |
| | | SSSPGASPGTSSTGSPGASPGTSSTGSPGTPGSGTASSSPGSSTPSGATGSPGASPGT |
| | | SSTGSPGTPGSGTASSSPGSSTPSGATGSPGSSPSASTGTGPGSSPSASTGTGPGSST |
| | | PSGATGSPGSSTPSGATGSPGASPGTSSTGSPGASPGTSSTGSPGASPGTSSTGSPGT |
| | | PGSGTASSSPGASPGTSSTGSPGASPGTSSTGSPGASPGTSSTGSPGSSPSASTGTGP |
| | | GTPGSGTASSSPGASPGTSSTGSPGASPGTSSTGSPGASPGTSSTGSPGSSTPSGATG |
| | | SPGSSTPSGATGSPGASPGTSSTGSPGTPGSGTASSSPGSSTPSGATGSPGSSTPSGA |
| | | TGSPGSSTPSGATGSPGSSPSASTGTGPGASPGTSSTGSPGASPGTSSTGSPGTPGSG |
| | | TASSSPGASPGTSSTGSPGASPGTSSTGSPGASPGTSSTGSPGASPGTSSTGSPGTPG |
| | | SGTASSSPGSSTPSGATGSPGTPGSGTASSSPGSSTPSGATGSPGTPGSGTASSSPGS |
| | | STPSGATGSPGSSTPSGATGSPGSSPSASTGTGPGSSPSASTGTGPGASPGTSSTGSP |
| | | GTPGSGTASSSPGSSTPSGATGSPGSSPSASTGTGPGSSPSASTGTGPGASPGTSSTG |
| | | SPGASPGTSSTGSPGSSTPSGATGSPGSSPSASTGTGPGASPGTSSTGSPGSSPSAST |
| | | GTGPGTPGSGTASSSPGSSTPSGATGSPGSSTPSGATGSPGASPGTSSTGSP |
| AM875 | 28 | GTSTEPSEGSAPGSEPATSGSETPGSPAGSPTSTEEGSTSSTAESPGPGTSTPESGSA |
| | | SPGSTSESPSGTAPGSTSESPSGTAPGTSTPESGSASPGTSTPESGSASPGSEPATSGS |
| | | ETPGTSESATPESGPGSPAGSPTSTEEGTSTEPSEGSAPGTSESATPESGPGTSTEPSE |
| | | GSAPGTSTEPSEGSAPGSPAGSPTSTEEGTSTEPSEGSAPGTSTEPSEGSAPGTSESA |
| | | TPESGPGTSESATPESGPGTSTEPSEGSAPGTSTEPSEGSAPGTSESATPESGPGTST |
| | | EPSEGSAPGSEPATSGSETPGSPAGSPTSTEEGSSTPSGATGSPGTPGSGTASSSPGS |
| | | STPSGATGSPGTSTEPSEGSAPGTSTEPSEGSAPGSEPATSGSETPGSPAGSPTSTEE |
| | | GSPAGSPTSTEEGTSTEPSEGSAPGASASGAPSTGGTSESATPESGPGSPAGSPTSTE |
| | | EGSPAGSPTSTEEGSTSSTAESPGPGSTSESPSGTAPGTSPSGESSTAPGTPGSGTAS |
| | | SSPGSSTPSGATGSPGSSPSASTGTGPGSEPATSGSETPGTSESATPESGPGSEPATS |
| | | GSETPGSTSSTAESPGPGSTSSTAESPGPGTSPSGESSTAPGSEPATSGSETPGSEPA |
| | | TSGSETPGTSTEPSEGSAPGSTSSTAESPGPGTSTPESGSASPGSTSESPSGTAPGTST |
| | | EPSEGSAPGTSTEPSEGSAPGTSTEPSEGSAPGSSTPSGATGSPGSSPSASTGTGPGA |
| | | SPGTSSTGSPGSEPATSGSETPGTSESATPESGPGSPAGSPTSTEEGSSTPSGATGSP |
| | | GSSPSASTGTGPGASPGTSSTGSPGTSESATPESGPGTSTEPSEGSAPGTSTEPSEGS |
| | | AP |
| AE912 | 29 | MAEPAGSPTSTEEGTPGSGTASSSPGSSTPSGATGSPGASPGTSSTGSPGSPAGSPT |
| | | STEEGTSESATPESGPGTSTEPSEGSAPGSPAGSPTSTEEGTSTEPSEGSAPGTSTEPS |
| | | EGSAPGTSESATPESGPGSEPATSGSETPGSEPATSGSETPGSPAGSPTSTEEGTSES |
| | | ATPESGPGTSTEPSEGSAPGTSTEPSEGSAPGSPAGSPTSTEEGTSTEPSEGSAPGTS |
| | | TEPSEGSAPGTSESATPESGPGTSTEPSEGSAPGTSESATPESGPGSEPATSGSETPG |
| | | TSTEPSEGSAPGTSTEPSEGSAPGTSESATPESGPGTSESATPESGPGSPAGSPTSTE |
| | | EGTSESATPESGPGSEPATSGSETPGTSESATPESGPGTSTEPSEGSAPGTSTEPSEG |
| | | SAPGTSTEPSEGSAPGTSTEPSEGSAPGTSTEPSEGSAPGTSTEPSEGSAPGSPAGSP |
| | | TSTEEGTSTEPSEGSAPGTSESATPESGPGSEPATSGSETPGTSESATPESGPGSEPA |
| | | TSGSETPGTSESATPESGPGTSTEPSEGSAPGTSESATPESGPGSPAGSPTSTEEGSP |
| | | AGSPTSTEEGSPAGSPTSTEEGTSESATPESGPGTSTEPSEGSAPGTSESATPESGPG |
| | | SEPATSGSETPGTSESATPESGPGSEPATSGSETPGTSESATPESGPGTSTEPSEGSA |
| | | PGSPAGSPTSTEEGTSESATPESGPGSEPATSGSETPGTSESATPESGPGSPAGSPTS |
| | | TEEGSPAGSPTSTEEGTSTEPSEGSAPGTSESATPESGPGTSESATPESGPGTSESAT |
| | | PESGPGSEPATSGSETPGSEPATSGSETPGSPAGSPTSTEEGTSTEPSEGSAPGTSTE |
| | | PSEGSAPGSEPATSGSETPGTSESATPESGPGTSTEPSEGSAP |
| AM923 | 30 | MAEPAGSPTSTEEGASPGTSSTGSPGSSTPSGATGSPGSSTPSGATGSPGTSTEPSE |
| | | GSAPGSEPATSGSETPGSPAGSPTSTEEGSTSSTAESPGPGTSTPESGSASPGSTSESP |
| | | SGTAPGSTSESPSGTAPGTSTPESGSASPGTSTPESGSASPGSEPATSGSETPGTSES |
| | | ATPESGPGSPAGSPTSTEEGTSTEPSEGSAPGTSESATPESGPGTSTEPSEGSAPGTS |
| | | TEPSEGSAPGSPAGSPTSTEEGTSTEPSEGSAPGTSTEPSEGSAPGTSESATPESGPG |
| | | TSESATPESGPGTSTEPSEGSAPGTSTEPSEGSAPGTSESATPESGPGTSTEPSEGSA |
| | | PGSEPATSGSETPGSPAGSPTSTEEGSSTPSGATGSPGTPGSGTASSSPGSSTPSGAT |
| | | GSPGTSTEPSEGSAPGTSTEPSEGSAPGSEPATSGSETPGSPAGSPTSTEEGSPAGSP |
| | | TSTEEGTSTEPSEGSAPGASASGAPSTGGTSESATPESGPGSPAGSPTSTEEGSPAGS |
| | | PTSTEEGSTSSTAESPGPGSTSESPSGTAPGTSPSGESSTAPGTPGSGTASSSPGSSTP |
| | | SGATGSPGSSPSASTGTGPGSEPATSGSETPGTSESATPESGPGSEPATSGSETPGST |
| | | SSTAESPGPGSTSSTAESPGPGTSPSGESSTAPGSEPATSGSETPGSEPATSGSETPG |
| | | TSTEPSEGSAPGSTSSTAESPGPGTSTPESGSASPGSTSESPSGTAPGTSTEPSEGSAP |
| | | GTSTEPSEGSAPGTSTEPSEGSAPGSSTPSGATGSPGSSPSASTGTGPGASPGTSSTG |
| | | SPGSEPATSGSETPGTSESATPESGPGSPAGSPTSTEEGSSTPSGATGSPGSSPSAST |
| | | GTGPGASPGTSSTGSPGTSESATPESGPGTSTEPSEGSAPGTSTEPSEGSAP |
| AM1318 | 31 | GTSTEPSEGSAPGSEPATSGSETPGSPAGSPTSTEEGSTSSTAESPGPGTSTPESGSA |
| | | SPGSTSESPSGTAPGSTSESPSGTAPGTSTPESGSASPGTSTPESGSASPGSEPATSGS |
| | | ETPGTSESATPESGPGSPAGSPTSTEEGTSTEPSEGSAPGTSESATPESGPGTSTEPSE |
| | | GSAPGTSTEPSEGSAPGSPAGSPTSTEEGTSTEPSEGSAPGTSTEPSEGSAPGTSESA |
| | | TPESGPGTSESATPESGPGTSTEPSEGSAPGTSTEPSEGSAPGTSESATPESGPGTST |
| | | EPSEGSAPGSEPATSGSETPGSPAGSPTSTEEGSSTPSGATGSPGTPGSGTASSSPGS |
| | | STPSGATGSPGTSTEPSEGSAPGTSTEPSEGSAPGSEPATSGSETPGSPAGSPTSTEE |
| | | GSPAGSPTSTEEGTSTEPSEGSAPGPEPTGPAPSGGSEPATSGSETPGTSESATPESG |
| | | PGSPAGSPTSTEEGTSESATPESGPGSPAGSPTSTEEGSPAGSPTSTEEGTSESATPE |
| | | SGPGSPAGSPTSTEEGSPAGSPTSTEEGSTSSTAESPGPGSTSESPSGTAPGTSPSGES |
| | | STAPGSTSESPSGTAPGSTSESPSGTAPGTSPSGESSTAPGTSTEPSEGSAPGTSESA |
| | | TPESGPGTSESATPESGPGSEPATSGSETPGTSESATPESGPGTSESATPESGPGTST |
| | | EPSEGSAPGTSESATPESGPGTSTEPSEGSAPGTSPSGESSTAPGTSPSGESSTAPGT |
| | | SPSGESSTAPGTSTEPSEGSAPGSPAGSPTSTEEGTSTEPSEGSAPGSSPSASTGTGP |
| | | GSSTPSGATGSPGSSTPSGATGSPGSSTPSGATGSPGSSTPSGATGSPGASPGTSSTG |
| | | SPGASASGAPSTGGTSPSGESSTAPGSTSSTAESPGPGTSPSGESSTAPGTSESATPE |
| | | SGPGTSTEPSEGSAPGTSTEPSEGSAPGSSPSASTGTGPGSSTPSGATGSPGASPGTS |
| | | STGSPGTSTPESGSASPGTSPSGESSTAPGTSPSGESSTAPGTSESATPESGPGSEPAT |
| | | SGSETPGTSTEPSEGSAPGSTSESPSGTAPGSTSESPSGTAPGTSTPESGSASPGSPA |
| | | GSPTSTEEGTSESATPESGPGTSTEPSEGSAPGSPAGSPTSTEEGTSESATPESGPGS |
| | | EPATSGSETPGSSTPSGATGSPGASPGTSSTGSPGSSTPSGATGSPGSTSESPSGTAP |
| | | GTSPSGESSTAPGSTSSTAESPGPGSSTPSGATGSPGASPGTSSTGSPGTPGSGTASS |
| | | SPGSPAGSPTSTEEGSPAGSPTSTEEGTSTEPSEGSAP |
| BC864 | 32 | GTSTEPSEPGSAGTSTEPSEPGSAGSEPATSGTEPSGSGASEPTSTEPGSEPATSGTE |
| | | PSGSEPATSGTEPSGSEPATSGTEPSGSGASEPTSTEPGTSTEPSEPGSAGSEPATSG |
| | | TEPSGTSTEPSEPGSAGSEPATSGTEPSGSEPATSGTEPSGTSTEPSEPGSAGTSTEPS |
| | | EPGSAGSEPATSGTEPSGSEPATSGTEPSGTSEPSTSEPGAGSGASEPTSTEPGTSEP |
| | | STSEPGAGSEPATSGTEPSGSEPATSGTEPSGTSTEPSEPGSAGTSTEPSEPGSAGSG |
| | | ASEPTSTEPGSEPATSGTEPSGSEPATSGTEPSGSEPATSGTEPSGSEPATSGTEPSG |
| | | TSTEPSEPGSAGSEPATSGTEPSGSGASEPTSTEPGTSTEPSEPGSAGSEPATSGTEP |
| | | SGSGASEPTSTEPGTSTEPSEPGSAGSGASEPTSTEPGSEPATSGTEPSGSGASEPTS |
| | | TEPGSEPATSGTEPSGSGASEPTSTEPGTSTEPSEPGSAGSEPATSGTEPSGSGASEP |
| | | TSTEPGTSTEPSEPGSAGSEPATSGTEPSGTSTEPSEPGSAGSEPATSGTEPSGTSTEP |
| | | SEPGSAGTSTEPSEPGSAGTSTEPSEPGSAGTSTEPSEPGSAGTSTEPSEPGSAGTST |
| | | EPSEPGSAGTSEPSTSEPGAGSGASEPTSTEPGTSTEPSEPGSAGTSTEPSEPGSAGT |
| | | STEPSEPGSAGSEPATSGTEPSGSGASEPTSTEPGSEPATSGTEPSGSEPATSGTEPS |
| | | GSEPATSGTEPSGSEPATSGTEPSGTSEPSTSEPGAGSEPATSGTEPSGSGASEPTST |
| | | EPGTSTEPSEPGSAGSEPATSGTEPSGSGASEPTSTEPGTSTEPSEPGSA |
| BD864 | 33 | GSETATSGSETAGTSESATSESGAGSTAGSETSTEAGTSESATSESGAGSETATSGS |
| | | ETAGSETATSGSETAGTSTEASEGSASGTSTEASEGSASGTSESATSESGAGSETAT |
| | | SGSETAGTSTEASEGSASGSTAGSETSTEAGTSESATSESGAGTSESATSESGAGSE |
| | | TATSGSETAGTSESATSESGAGTSTEASEGSASGSETATSGSETAGSETATSGSETA |
| | | GTSTEASEGSASGSTAGSETSTEAGTSESATSESGAGTSTEASEGSASGSETATSGS |
| | | ETAGSTAGSETSTEAGSTAGSETSTEAGSETATSGSETAGTSESATSESGAGTSESA |
| | | TSESGAGSETATSGSETAGTSESATSESGAGTSESATSESGAGSETATSGSETAGSE |
| | | TATSGSETAGTSTEASEGSASGSTAGSETSTEAGSETATSGSETAGTSESATSESGA |
| | | GSTAGSETSTEAGSTAGSETSTEAGSTAGSETSTEAGTSTEASEGSASGSTAGSETS |
| | | TEAGSTAGSETSTEAGTSTEASEGSASGSTAGSETSTEAGSETATSGSETAGTSTEA |
| | | SEGSASGTSESATSESGAGSETATSGSETAGTSESATSESGAGTSESATSESGAGSE |
| | | TATSGSETAGTSESATSESGAGSETATSGSETAGTSTEASEGSASGTSTEASEGSAS |
| | | GSTAGSETSTEAGSTAGSETSTEAGSETATSGSETAGTSESATSESGAGTSESATSE |
| | | SGAGSETATSGSETAGSETATSGSETAGSETATSGSETAGTSTEASEGSASGTSESA |
| | | TSESGAGSETATSGSETAGSETATSGSETAGTSESATSESGAGTSESATSESGAGSE |
| | | TATSGSETA |
| AE911 | 34 | AEPAGSPTSTEEGTPGSGTASSSPGSSTPSGATGSPGASPGTSSTGSPGSPAGSPTST |
| | | EEGTSESATPESGPGTSTEPSEGSAPGSPAGSPTSTEEGTSTEPSEGSAPGTSTEPSE |
| | | GSAPGTSESATPESGPGSEPATSGSETPGSEPATSGSETPGSPAGSPTSTEEGTSESA |
| | | TPESGPGTSTEPSEGSAPGTSTEPSEGSAPGSPAGSPTSTEEGTSTEPSEGSAPGTST |
| | | EPSEGSAPGTSESATPESGPGTSTEPSEGSAPGTSESATPESGPGSEPATSGSETPGT |
| | | STEPSEGSAPGTSTEPSEGSAPGTSESATPESGPGTSESATPESGPGSPAGSPTSTEE |
| | | GTSESATPESGPGSEPATSGSETPGTSESATPESGPGTSTEPSEGSAPGTSTEPSEGS |
| | | APGTSTEPSEGSAPGTSTEPSEGSAPGTSTEPSEGSAPGTSTEPSEGSAPGSPAGSPT |
| | | STEEGTSTEPSEGSAPGTSESATPESGPGSEPATSGSETPGTSESATPESGPGSEPAT |
| | | SGSETPGTSESATPESGPGTSTEPSEGSAPGTSESATPESGPGSPAGSPTSTEEGSPA |
| | | GSPTSTEEGSPAGSPTSTEEGTSESATPESGPGTSTEPSEGSAPGTSESATPESGPGS |
| | | EPATSGSETPGTSESATPESGPGSEPATSGSETPGTSESATPESGPGTSTEPSEGSAP |
| | | GSPAGSPTSTEEGTSESATPESGPGSEPATSGSETPGTSESATPESGPGSPAGSPTST |
| | | EEGSPAGSPTSTEEGTSTEPSEGSAPGTSESATPESGPGTSESATPESGPGTSESATP |
| | | ESGPGSEPATSGSETPGSEPATSGSETPGSPAGSPTSTEEGTSTEPSEGSAPGTSTEP |
| | | SEGSAPGSEPATSGSETPGTSESATPESGPGTSTEPSEGSAP |
| AE146 | 35 | GGTSESATPESGPGTSTEPSEGSAPGTSTEPSEGSAPGTSESATPESGPGTSTEPSEG |
| | | SAPGTSTEPSEGSAPGTSESATPESGPGTSTEPSEGSAPGTSTEPSEGSAPGTSTEPS |
| | | EGSAPGSPAGSPTSTEEGTSTEPSEGSAPG |
| AE48.1 | 36 | AEPAGSPTSTEEGTPGSGTASSSPGSSTPSGATGSPGASPGTSSTGS |
| AM48.1 | 37 | AEPAGSPTSTEEGASPGTSSTGSPGSSTPSGATGSPGSSTPSGATGS |
| AE912.1 | 38 | AEPAGSPTSTEEGTPGSGTASSSPGSSTPSGATGSPGASPGTSSTGSPGSPAGSPTST |
| | | EEGTSESATPESGPGTSTEPSEGSAPGSPAGSPTSTEEGTSTEPSEGSAPGTSTEPSE |
| | | GSAPGTSESATPESGPGSEPATSGSETPGSEPATSGSETPGSPAGSPTSTEEGTSESA |
| | | TPESGPGTSTEPSEGSAPGTSTEPSEGSAPGSPAGSPTSTEEGTSTEPSEGSAPGTST |
| | | EPSEGSAPGTSESATPESGPGTSTEPSEGSAPGTSESATPESGPGSEPATSGSETPGT |
| | | STEPSEGSAPGTSTEPSEGSAPGTSESATPESGPGTSESATPESGPGSPAGSPTSTEE |
| | | GTSESATPESGPGSEPATSGSETPGTSESATPESGPGTSTEPSEGSAPGTSTEPSEGS |
| | | APGTSTEPSEGSAPGTSTEPSEGSAPGTSTEPSEGSAPGTSTEPSEGSAPGSPAGSPT |
| | | STEEGTSTEPSEGSAPGTSESATPESGPGSEPATSGSETPGTSESATPESGPGSEPAT |
| | | SGSETPGTSESATPESGPGTSTEPSEGSAPGTSESATPESGPGSPAGSPTSTEEGSPA |
| | | GSPTSTEEGSPAGSPTSTEEGTSESATPESGPGTSTEPSEGSAPGTSESATPESGPGS |
| | | EPATSGSETPGTSESATPESGPGSEPATSGSETPGTSESATPESGPGTSTEPSEGSAP |
| | | GSPAGSPTSTEEGTSESATPESGPGSEPATSGSETPGTSESATPESGPGSPAGSPTST |
| | | EEGSPAGSPTSTEEGTSTEPSEGSAPGTSESATPESGPGTSESATPESGPGTSESATP |
| | | ESGPGSEPATSGSETPGSEPATSGSETPGSPAGSPTSTEEGTSTEPSEGSAPGTSTEP |
| | | SEGSAPGSEPATSGSETPGTSESATPESGPGTSTEPSEGSAP |
| AE912.2 | 39 | AEPAGSPTSTEEGTPGSGTASSSPGSSTPSGATGSPGASPGTSSTGSPGSPAGSPTST |
| | | EEGTSESATPESGPGTSTEPSEGSAPGSPAGSPTSTEEGTSTEPSEGSAPGTSTEPSE |
| | | GSAPGTSESATPESGPGSEPATSGSETPGSEPATSGSETPGSPAGSPTSTEEGTSESA |
| | | TPESGPGTSTEPSEGSAPGTSTEPSEGSAPGSPAGSPTSTEEGTSTEPSEGSAPGTST |
| | | EPSEGSAPGTSESATPESGPGTSTEPSEGSAPGTSESATPESGPGSEPATSGSETPGT |
| | | STEPSEGSAPGTSTEPSEGSAPGTSESATPESGPGTSESATPESGPGSPAGSPTSTEE |
| | | GTSESATPESGPGSEPATSGSETPGTSESATPESGPGTSTEPSEGSAPGTSTEPSEGS |
| | | APGTSTEPSEGSAPGTSTEPSEGSAPGTSTEPSEGSAPGTSTEPSEGSAPGSPAGSPT |
| | | STEEGTSTEPSEGSAPGTSESATPESGPGSEPATSGSETPGTSESATPESGPGSEPAT |
| | | SGSETPGTSESATPESGPGTSTEPSEGSAPGTSESATPESGPGSPAGSPTSTEEGSPA |
| | | GSPTSTEEGSPAGSPTSTEEGTSESATPESGPGTSTEPSEGSAPGTSESATPESGPGS |
| | | EPATSGSETPGTSESATPESGPGSEPATSGSETPGTSESATPESGPGTSTEPSEGSAP |
| | | GSPAGSPTSTEEGTSESATPESGPGSEPATSGSETPGTSESATPESGPGSPAGSPTST |
| | | EEGSPAGSPTSTEEGTSTEPSEGSAPGTSESATPESGPGTSESATPESGPGTSESATP |
| | | ESGPGSEPATSGSETPGSEPATSGSETPGSPAGSPTSTEEGTSTEPSEGSAPGTSTEP |
| | | SEGSAPGSEPATSGSETPGTSESATPESGPGTSTEPSEGSAPG |
| AE146.1 | 40 | TSESATPESGPGTSTEPSEGSAPGTSTEPSEGSAPGTSESATPESGPGTSTEPSEGSA |
| | | PGTSTEPSEGSAPGTSESATPESGPGTSTEPSEGSAPGTSTEPSEGSAPGTSTEPSEG |
| | | SAPGSPAGSPTSTEEGTSTEPSEGSAPG |

In those embodiments wherein the XTEN component of the hGH-XTEN fusion protein has less than 100% of its amino acids consisting of 4, 5, or 6 types of amino acid selected from glycine (G), alanine (A), serine (S), threonine (T), glutamate (E) and proline (P), or less than 100% of the sequence consisting of the XTEN sequences of Table 2, the other amino acid residues of the XTEN are selected from any of the other 14 natural L-amino acids, but are preferentially selected from hydrophilic amino acids such that the XTEN sequence contains at least about 90%, 91%, 92%, 93%, 94%, 95%, 96%, 97%, 98%, or at least about 99% hydrophilic amino acids. The XTEN amino acids that are not glycine (G), alanine (A), serine (S), threonine (T), glutamate (E) and proline (P) are either interspersed throughout the XTEN sequence, are located within or between the sequence motifs, or are concentrated in one or more short stretches of the XTEN sequence, e.g., to create a linker between the XTEN and the hGH components. In such cases where the XTEN component of the hGH-XTEN comprises amino acids other than glycine (G), alanine (A), serine (S), threonine (T), glutamate (E) and proline (P), it is preferred that less than about 2% or less than about 1% of the amino acids be hydrophobic residues such that the resulting sequences generally lack secondary structure, e.g., not having more than 2% alpha helices or 2% beta-sheets, as determined by the methods disclosed herein. Hydrophobic residues that are less favored in construction of XTEN include tryptophan, phenylalanine, tyrosine, leucine, isoleucine, valine, and methionine. Additionally, one can design the XTEN sequences to contain less than 5% or less than 4% or less than 3% or less than 2% or less than 1% or none of the following amino acids: cysteine (to avoid disulfide formation and oxidation), methionine (to avoid oxidation), asparagine and glutamine (to avoid desamidation). Thus, in some embodiments, the XTEN component of the hGH-XTEN fusion protein comprising other amino acids in addition to glycine (G), alanine (A), serine (S), threonine (T), glutamate (E) and proline (P) have a sequence with less than 5% of the residues contributing to alpha-helices and beta-sheets as measured by the Chou-Fasman algorithm and have at least 90%, or at least about 95% or more random coil formation as measured by the GOR algorithm.

4. XTEN Segments

In one embodiment, the invention provides an isolated hGH-XTEN fusion protein wherein the cumulative length of the XTEN component is greater than about 100 to about 3000 amino acid residues containing at least one polypeptide sequence segment selected from Table 2 (and Tables 8, 9, 10, 11, and 12 of Schellenberger et al. WO10/144502A2, which is incorporated herein by reference in its entirety) and wherein at least about 90%, or at least about 91%, or at least about 92%, or at least about 93%, or at least about 94%, or at least about 95%, or at least about 96%, or at least about 97%, or at least about 98% or more of the remainder of the XTEN sequence by and large contains hydrophilic amino acids and less than about 2% of the remainder of the XTEN consists of hydrophobic or aromatic amino acids, or cysteine. In some embodiments, the XTEN contains multiple segments wherein the segments are identical or different (see Schellenberger et al. WO10/144502A2; Cleland et al. U.S. Ser. No. 13/829,369; and Cleland et al. WO13/184216, each of which is incorporated herein by reference in its entirety).

5. N-Terminal XTEN Expression-Enhancing Sequences

In some embodiments, the invention provides a short-length XTEN sequence incorporated as the N-terminal portion of the hGH-XTEN fusion protein. The expression of the fusion protein is enhanced in a host cell transformed with a suitable expression vector comprising an optimized N-terminal leader polynucleotide sequence (that encodes the N-terminal XTEN) incorporated into the polynucleotide encoding the binding fusion protein. It has been discovered, as described in Examples 14-17 of Schellenberger et al. WO10/144502A2 (which is incorporated herein by reference in its entirety), that a host cell transformed with such an expression vector comprising an optimized N-terminal leader sequence (NTS) in the binding fusion protein gene results in greatly-enhanced expression of the fusion protein compared to the expression of a corresponding fusion protein from a polynucleotide not comprising the NTS, and obviates the need for incorporation of a non-XTEN leader sequence used to enhance expression (see Schellenberger et al. WO10/144502A2; Cleland et al. U.S. Ser. No. 13/829,369; and Cleland et al. WO13/184216, each of which is incorporated herein by reference in its entirety).

In one embodiment, the N-terminal XTEN polypeptide of the hGH-XTEN comprises a sequence that exhibits at least about 80%, more preferably at least about 90%, more preferably at least about 91%, more preferably at least about 92%, more preferably at least about 93%, more preferably at least about 94%, more preferably at least about 95%, more preferably at least about 96%, more preferably at least about 97%, more preferably at least about 98%, more preferably at least 99%, or exhibits 100% sequence identity to the amino acid sequence of AE48, AE48.1, AM48, or AM48.1, the respective amino acid sequences of which are as follows:

AE48:
(SEQ ID NO: 13)
MAEPAGSPTSTEEGTPGSGTASSSPGSSTPSGATGSPGASPGTSSTGS
AE48.1:
(SEQ ID NO: 36)
AEPAGSPTSTEEGTPGSGTASSSPGSSTPSGATGSPGASPGTSSTGS
AM48:
(SEQ ID NO: 14)
MAEPAGSPTSTEEGASPGTSSTGSPGSSTPSGATGSPGSSTPSGATGS
AM48.1:
(SEQ ID NO: 37)
AEPAGSPTSTEEGASPGTSSTGSPGSSTPSGATGSPGSSTPSGATGS.

In another embodiment, the N-terminal XTEN polypeptide of the hGH-XTEN comprises a sequence exhibiting at least 90% identity to AE48, AM48 or AE912, as described herein, wherein the N-terminal M residue is absent (e.g., AE48.1—SEQ ID NO:36; AM48.1—SEQ ID NO:37; and AE912.1—SEQ ID NO:38). In an additional embodiment, the C-terminal XTEN poly peptide of the hGH-XTEN comprises a sequence exhibiting at least 90% identity to AE146, as described herein, (e.g., AE146—SEQ ID NO:35; or AE146.1—SEQ ID NO:40).

In another embodiment, the short-length N-terminal XTEN is linked to an XTEN of longer length to form the N-terminal region of the hGH-XTEN fusion protein, wherein the polynucleotide sequence encoding the short-length N-terminal XTEN confers the property of enhanced expression in the host cell, and wherein the long length of the expressed XTEN contributes to the enhanced properties of the XTEN carrier in the fusion protein, as described above. In some embodiments, the N-terminal XTEN polypeptide with long length exhibits at least about 80%, or at least about 90%, or at least about 91%, or at least about 92%, or at least about 93%, or at least about 94%, or at least about 95%, or at least about 96%, or at least about 97%, or at least about 98%, or at least 99%, or exhibits 100% sequence identity to an amino acid sequence selected from the group consisting of the sequences AE624, AE911, AE912, and AM923.

6. Net Charge

In other embodiments, the XTEN polypeptides have an unstructured characteristic imparted by incorporation of amino acid residues with a net charge and/or reducing the proportion of hydrophobic amino acids in the XTEN sequence. The overall net charge and net charge density is controlled by modifying the content of charged amino acids in the XTEN sequences. In some embodiments, the net charge density of the XTEN of the compositions may be above +0.1 or below −0.1 charges/residue. In other embodiments, the net charge of a XTEN can be about 0%, about 1%, about 2%, about 3%, about 4%, about 5%, about 6%, about 7%, about 8%, about 9%, about 10% about 11%, about 12%, about 13%, about 14%, about 15%, about 16%, about 17%, about 18%, about 19%, or about 20% or more (see Schellenberger et al. WO10/144502A2; Cleland et al. U.S. Ser. No. 13/829,369; and Cleland et al. PCT/US2013/031673, each of which is incorporated herein by reference in its entirety).

7. Low Immunogenicity

In another aspect, the invention provides compositions in which the XTEN sequences have a low degree of immunogenicity or are substantially non-immunogenic. Several factors can contribute to the low immunogenicity of XTEN, e.g., the non-repetitive sequence, the unstructured conformation, the high degree of solubility, the low degree or lack of self-aggregation, the low degree or lack of proteolytic sites within the sequence, and the low degree or lack of epitopes in the XTEN sequence (see Schellenberger et al. WO10/144502A2; Cleland et al. U.S. Ser. No. 13/829,369; and Cleland et al. PCT/US2013/031673, each of which is incorporated herein by reference in its entirety).

8. Increased Hydrodynamic Radius

In another aspect, the present invention provides XTEN in which the XTEN polypeptides have a high hydrodynamic radius that confers a corresponding increased Apparent Molecular Weight to the hGH-XTEN fusion protein incorporating the XTEN. As detailed in Example 37 of Schellenberger et al. WO10/144502A2, the linking of XTEN to GH sequences results in hGH-XTEN compositions that can have increased hydrodynamic radii, increased Apparent Molecular Weight, and increased Apparent Molecular Weight Factor compared to a GH not linked to an XTEN (see Schellenberger et al. WO10/144502A2; Cleland et al. U.S. Ser. No. 13/829,369; and Cleland et al. WO13/184216, each of which is incorporated herein by reference in its entirety).

V). hGH-XTEN Structural Configurations and Properties

The human growth hormone (GH) of the subject compositions are not limited to native, full-length polypeptides, but also include recombinant versions as well as biologically and/or pharmacologically active variants or fragments thereof. For example, it will be appreciated that various amino acid deletions, insertions and substitutions can be made in the GH to create variants without departing from the spirit of the invention with respect to the biological activity or pharmacologic properties of the GH. Examples of conservative substitutions for amino acids in polypeptide sequences are shown in Table 3. However, in embodiments of the hGH-XTEN in which the sequence identity of the GH is less than 100% compared to a specific sequence disclosed herein, the invention contemplates substitution of any of the other 19 natural L-amino acids for a given amino acid residue of the given GH, which may be at any position within the sequence of the GH, including adjacent amino acid residues. If any one substitution results in an undesirable change in biological activity, then one of the alternative amino acids can be employed and the construct evaluated by the methods described herein, or using any of the techniques and guidelines for conservative and non-conservative mutations set forth, for instance, in U.S. Pat. No. 5,364,934, the contents of which is incorporated by reference in its entirety, or using methods generally known in the art. In addition, variants can include, for instance, polypeptides wherein one or more amino acid residues are added or deleted at the N- or C-terminus of the full-length native amino acid sequence of a GH that retains some if not all of the biological activity of the native peptide.

TABLE 3
Exemplary conservative amino acid substitutions

| Original Residue | Exemplary Substitutions |
|---|---|
| Ala (A) | val; leu; ile |
| Arg (R) | lys; gin; asn |
| Asn (N) | gin; his; Iys; arg |
| Asp (D) | glu |
| Cys (C) | ser |
| Gln (Q) | asn |
| Glu (E) | asp |
| Gly (G) | pro |
| His (H) | asn: gin: Iys: arg |
| xIle (I) | leu; val; met; ala; phe: norleucine |
| Leu (L) | norleucine: ile: val; met; ala: phe |
| Lys (K) | arg: gin: asn |
| Met (M) | leu; phe; ile |
| Phe (F) | leu: val: ile; ala |
| Pro (P) | gly |
| Ser (S) | thr |
| Thr (T) | ser |
| Trp (W) | tyr |
| Tyr (Y) | trp: phe: thr: ser |
| Val (V) | ile; leu; met; phe; ala; norleucine |

(a) Fusion Protein Configurations

The invention provides fusion protein compositions with the GH and XTEN components linked in specific N- to C-terminus configurations. In some embodiments, one or more GHs are linked to one or more XTENs, either at the N-terminus or at the C-terminus, with or without a spacer, to form a block copolymer, and the sequential arrangement of the GHs and the XTENs in the fusion protein are the same as the configuration known in the block copolymer chemistry. When there is more than one GH, XTEN, or spacer, each of the GH, the XTEN, or the spacer have the same or different sequences, and the GHs and/or XTENs are linked either continuously or alternately (regular or irregular). Thus, in all of the formulae provided herein, when there is more than one GH, XTEN, or spacer, each of the GH, XTEN, and spacer are the same or different. In some embodiments, the fusion protein is a monomeric fusion protein with a GH linked to one XTEN polypeptide. In other embodiments, the fusion protein is a monomeric fusion protein with a GH linked to two or more XTEN polypeptides. In still other embodiments, the fusion protein is a monomeric fusion protein with two or more GH linked to one XTEN polypeptide. In still other embodiments, the fusion protein is a monomeric fusion protein with two or more GH linked to two or more XTEN polypeptide. Table 4 provides non-limiting examples of configurations that are encompassed by the invention; numerous other variations will be apparent to the ordinarily skilled artisan, including the incorporation the spacer and cleavage sequences disclosed herein or known in the art.

TABLE 4
hGH-XTEN configurations

| Components* | Configuration** |
|---|---|
| Single GH; Single XTEN | GH-XTEN |
| | XTEN-GH |
| Single GH; Multiple XTEN | XTEN-GH-XTEN |
| | GH-XTEN-XTEN |
| | XTEN-XTEN-GH |
| | XTEN-GH-XTEN-XTEN |
| | XTEN-XTEN-GH-XTEN |
| | XTEN-XTEN-GH-XTEN |
| Multiple GH, Single XTEN | GH-XTEN-GH |
| | XTEN-GH-GH |
| | GH-GH-XTEN |
| | GH-XTEN-GH-GH |
| Multiple GH; Multiple XTEN | GH-XTEN-GH-XTEN |
| | XTEN-GH-XTEN-GH |
| | XTEN-XTEN-GH-XTEN-GH |
| | XTEN-XTEN-GH-GH |
| | GH-XTEN-XTEN-GH |
| | GH-GH-XTEN-XTEN |
| | GH-GH-XTEN-XTEN-GH |
| | GH-XTEN-GH-XTEN-GH |

*Characterized as single for 1 component or multiple for 2 or more of that component
**Reflects N- to C-terminus configuration of the growth factor and XTEN components

The invention contemplates fusion proteins compositions that are in a configuration shown in Table 4 and that retain at least a portion of the biological activity of the corresponding GH not linked to the XTEN. In other embodiments, the GH component either becomes biologically active or has an increase in activity upon its release from the XTEN by cleavage of an optional cleavage sequence incorporated within spacer sequences into the hGH-XTEN, described more fully below.

In one embodiment of the hGH-XTEN composition, the invention provides a fusion protein of formula I:

(XTEN)x-GH-(XTEN)y I

wherein independently for each occurrence, GH is a human growth hormone; x is either 0 or 1 and y is either 0 or 1 wherein x+y≧1; and XTEN is an extended recombinant polypeptide.

In another embodiment of the hGH-XTEN composition, the invention provides a fusion protein of formula II:

(XTEN)x-(GH)-(S)y-(XTEN)y II

wherein independently for each occurrence, GH is a human growth hormone; S is a spacer sequence having between 1 to about 50 amino acid residues that can optionally include a cleavage sequence; x is either 0 or 1 and y is either 0 or 1 wherein x+y≧1; and XTEN is an extended recombinant polypeptide.

In another embodiment, the invention provides an isolated fusion protein, wherein the fusion protein is of formula III:

(GH)-(S)x-(XTEN)-(S)y-(GH)-(S)z-(XTEN)z III

wherein independently for each occurrence, GH is a human growth hormone; S is a spacer sequence having between 1 to about 50 amino acid residues that can optionally include a cleavage sequence; x is either 0 or 1; y is either 0 or 1; z is either 0 or 1; and XTEN is an extended recombinant polypeptide.

In another embodiment, the invention provides an isolated fusion protein, wherein the fusion protein is of formula IV:

(XTEN)x-(S)y-(GH)-(S)z-(XTEN)-(GH) IV

wherein independently for each occurrence, GH is a human growth hormone; S is a spacer sequence having between 1 to about 50 amino acid residues that can optionally include a cleavage sequence; x is either 0 or 1; y is either 0 or 1; z is either 0 or 1; and XTEN is an extended recombinant polypeptide.

In another embodiment, the invention provides an isolated fusion protein, wherein the fusion protein is of formula V:

(GH)x-(S)x-(GH)-(S)y-(XTEN) V

wherein independently for each occurrence, GH is a growth hormone; S is a spacer sequence having between 1 to about 50 amino acid residues that can optionally include a cleavage sequence; x is either 0 or 1; y is either 0 or 1; and XTEN is an extended recombinant polypeptide.

In another embodiment, the invention provides an isolated fusion protein, wherein the fusion protein is of formula VI:

(XTEN)-(S)x-(GH)-(S)y-(GH) VI

wherein independently for each occurrence, GH is a growth hormone; S is a spacer sequence having between 1 to about 50 amino acid residues that can optionally include a cleavage sequence; x is either 0 or 1; y is either 0 or 1; and XTEN is an extended recombinant polypeptide.

In another embodiment, the invention provides an isolated fusion protein, wherein the fusion protein is of formula VII:

(XTEN)-(S)x-(GH)-(S)y-(GH)-(XTEN) VII

wherein independently for each occurrence, GH is a growth hormone; S is a spacer sequence having between 1 to about 50 amino acid residues that can optionally include a cleavage sequence; x is either 0 or 1; y is either 0 or 1; and XTEN is an extended recombinant polypeptide.

In another embodiment, the invention provides an isolated fusion protein, wherein the fusion protein is of formula VIII:

((S)m-(GH)x-(S)n-(XTEN)y-(S)o)t VIII

wherein t is an integer that is greater than 0 (1, 2, 3, etc.); independently each of m, n, o, x, and y is an integer (0, 1, 2, 3, etc.), GH is a growth hormone; S is an spacer, optionally comprising a cleavage site; and XTEN is an extended recombinant polypeptide, with the proviso that: (1) x+y>1, (2) when t=1, x>0 and y>0, (3) when there is more than one GH, S, or XTEN, each GH, XTEN, or S are the same or are independently different; and (4) when t>1, each m, n, o, x, or y within each subunit are the same or are independently different.

In another embodiment, the invention provides an isolated fusion protein, wherein the fusion protein is of formula IX:

(XTEN)x-(S)x-(GH)-(S)y-(XTEN)y IX

wherein independently for each occurrence, GH is a human growth hormone; S is a spacer sequence having between 1 to about 50 amino acid residues that can optionally include a cleavage sequence; x is either 0 or 1 and y is either 0 or 1 wherein x+y>1; and XTEN is an extended recombinant polypeptide.

Any spacer sequence group is optional in the fusion proteins encompassed by the invention. The spacer is provided to enhance expression of the fusion protein from a host cell or to decrease steric hindrance such that the GH component may assume its desired tertiary structure and/or interact appropriately with its target receptor. For spacers and methods of identifying desirable spacers, see, for example, George, et al. (2003) Protein Engineering 15:871-879, specifically incorporated by reference herein. In one embodiment, the spacer comprises one or more peptide sequences that are between 1-50 amino acid residues in length, or about 1-25 residues, or about 1-10 residues in length. Spacer sequences, exclusive of cleavage sites, can comprise any of the 20 natural L amino acids, and will preferably comprise hydrophilic amino acids that are sterically unhindered that can include, but not be limited to, glycine (G), alanine (A), serine (S), threonine (T), glutamate (E) and proline (P). In some cases, the spacer can be polyglycines or polyalanines, or is predominately a mixture of combinations of glycine and alanine residues. The spacer polypeptide exclusive of a cleavage sequence is largely to substantially devoid of secondary structure; e.g., less than about 10%, or less than about 5% as determined by the Chou-Fasman and/or GOR algorithms. In one embodiment, one or both spacer sequences in a hGH-XTEN fusion protein composition each further contains a cleavage sequence, which are identical or different, wherein the cleavage sequence may be acted on by a protease to release the GH from the fusion protein.

In one embodiment, a GH incorporated into a hGH-XTEN fusion protein has a sequence that exhibits at least about 80% sequence identity to a sequence shown as SEQ ID NO:41, alternatively at least about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%, or about 100% sequence identity as compared with the sequence of SEQ ID NO:41. The GH of the foregoing embodiment can be evaluated for activity using assays or measured or determined parameters as described herein, and those sequences that retain at least about 40%, or about 50%, or about 55%, or about 60%, or about 70%, or about 80%, or about 90%, or about 95% or more activity compared to the corresponding native GH sequence would be considered suitable for inclusion in the subject hGH-XTEN. The GH found to retain a suitable level of activity can be linked to one or more XTEN polypeptides described hereinabove. In one embodiment, a GH found to retain a suitable level of activity can be linked to one or more XTEN polypeptides having at least about 80% sequence identity to a sequence from Table 3, alternatively at least about 81%, 82%, 83%, 84%, 85%, 86%, 87%, 88%, 89%, 90%, 91%, 92%, 93%, 94%, 95%, 96%, 97%, 98%, 99%, or about 100% sequence identity as compared with a sequence of Table 3, resulting in a chimeric fusion protein.

Non-limiting examples of sequences of fusion proteins containing a single GH linked to a single XTEN are presented in Table 35 of Schellenberger et al. WO10/144502A2, which is incorporated herein by reference in its entirety. In one embodiment, a hGH-XTEN composition would comprise a fusion protein having at least about 80% sequence identity to a hGH-XTEN from Table 35 of Schellenberger et al. WO10/144502A2 (which is incorporated herein by reference in its entirety), alternatively at least about 81%, 82%, 83%, 84%, 85%, 86%, 87%, 88%, 89%, 90%, 91%, 92%, 93%, 94%, 95%, 96%, 97%, 98%, 99%, or about 100% sequence identity as compared with a hGH-XTEN from Table 35 of Schellenberger et al. WO10/144502A2, which is incorporated herein by reference in its entirety. Non-limiting examples of sequences of fusion proteins containing two molecules of XTEN linked to one or more GH are presented in Table 36 of Schellenberger et al. WO10/144502A2 (which is incorporated herein by reference in its entirety), but the invention also contemplates substitution of other GH with sequences exhibiting at least about 90% sequence identity to the sequence of SEQ ID NO:41 linked to one or two XTEN, which may be the same or different, exhibiting at least about 90% sequence identity to sequences selected from Table 2. Non-limiting examples of hGH-XTEN comprising GH, XTEN, and spacer amino acids are presented in Table 37 of Schellenberger et al. WO10/144502A2, which is incorporated herein by reference in its entirety. (see also Schellenberger et al. WO10/144502A2; Cleland et al. U.S. Ser. No. 13/829,369; and Cleland et al. WO13/184216, each of which is incorporated herein by reference in its entirety).

### VI). Uses of the Compositions of the Present Invention

Most processes involved in growth of the body are regulated by multiple peptides and hormones, and such peptides and hormones, as well as analogues thereof, have found utility in the treatment of growth hormone-related diseases, disorders and conditions. However, the use of commercially-available growth hormones to treat pediatric patients, has met with less than optimal success in the management of pediatric patients afflicted with such diseases, disorders and conditions. In particular, dose optimization and frequency of dosing is important for peptide and hormone biologics used in the treatment of growth hormone-related diseases and disorders in pediatric patients. The fact that growth hormone has a short half-life (e.g., usually less than 4 hours when administered subcutaneously), necessitates frequent (e.g., daily) dosing in order to achieve clinical benefit, which results in difficulties in the management of such pediatric patients. Non-compliance with daily growth hormone (GH) injections can lead to loss of treatment effects.

When compared to the current treatment protocol for recombinant hGH (rhGH), the benefit of an hGH-XTEN fusion protein to pediatric PGHD patients may include a substantial reduction in the number and frequency of injections. For example, in the Phase 2a stage of the clinical trial (see Example 2), pediatric PGHD patients will receive significantly fewer total injections (e.g., 6 total injections, once per month for 6 months) of an hGH-XTEN fusion protein compared to the 180 total injections of rhGH that these patients would have received on daily rhGH therapy over 6 months) than a pediatric patient undergoing daily rhGH therapy would receive over the same time period. The frequency of injection with rhGH in current clinical practice often leads to a lack of compliance. Compliance with daily therapy is crucial in order to realize the full potential for normal growth (Rosenfeld, R. G. & Bakker, B. (2008). Endocr Pract 14, 143-54; Desrosiers, P. et al. (2005). Pediatr Endocrinol Rev 2 Suppl 3, 327-31). An hGH-XTEN fusion protein is expected to provide the advantage of non-daily (e.g., bi-weekly, weekly, every two weeks, every three weeks, or monthly) administration to children with PGHD, and to offer a safe alternative to the current daily injections. An hGH product administered less frequently than daily rhGH therapy may provide greater compliance and therefore better long-term treatment outcomes for PGHD children.

In one aspect, the invention provides a method for achieving a beneficial effect in a disease, disorder or condition mediated by GH including, but not limited to growth hormone deficiency in a pediatric human patient. In another aspect, the invention provides a method for achieving a beneficial effect in a disease, disorder or condition mediated by GH including, but not limited to growth hormone deficiency in pediatric patients. The beneficial effect includes, without limitation, treating, mediating, or ameliorating a GH-related disease, deficiency, disorder or condition. The present invention addresses disadvantages and/or limitations of GH that have a relatively short terminal half-life and/or a narrow therapeutic window.

1. Pediatric Growth Hormone Deficiency (PGHD)

“Pediatric Growth Hormone Deficiency” or “PGHD” as used herein refers to a disease, deficiency, disorder or condition in a human pediatric patient that would benefit from treatment with growth hormone. PGHD includes disorders that are classified based on the source of the GH deficiency (e.g., pituitary PGHD, hypothalamic PGHD, functional PGHD, and idiopathic PGHD). Pituitary or “classic” PGHD is the incapacity of the pituitary to produce growth hormone. “Hypothalamic PGHD” is the failure of the hypothalamus to produce and/or transmit the neuroendocrine messaging hormone, growth hormone releasing hormone (GHRH), which directs a properly functioning pituitary to produce GH; “functional PGHD” is the failure of other hormone and of metabolic functions related to the failure of the pituitary to produce, uptake, and/or utilize GH. PGHD also includes, without limitation, idiopathic short stature, Turner syndrome, Prader Willi syndrome, small for gestational age (SGA), growth failure as a result of a deficiency in the short stature homeobox-containing gene (SHOX deficiency); and chronic kidney disease (CKD). The PGHD may be congenital or acquired in nature.

PGHD may also occur as a result of intrauterine growth retardation, congenital hypopituitarism or acquired hypopituitarism (including hypopituitarism caused by a tumor, e.g., craniopharyngioma); small for gestational age, developmental defects in or near the pituitary gland; genetic problems with the production of GH; Prader-Willi syndrome; Turner syndrome; idiopathic short stature; intrauterine growth retardation; midline facial defects; and damage to the pituitary gland or the surrounding area due to tumors, infection, radiation treatment, or severe head injury.

PGHD may be classified based on the stage of life the GH deficiency became manifest. For example, an adolescent may have PGHD that is a continuation of childhood onset PGHD (including child-onset PGHD and child-onset idiopathic PGHD), which began in infancy or pre-adolescent childhood. The causes of childhood-onset PGHD are provided above. Adolescents who survived brain tumors as pre-adolescent children may be at risk of developing PGHD from the effects of surgery, cranial radiation or chemotherapy. PGHD can develop in an adolescent, i.e., childhood-onset PGHD, who was not diagnosed as being GH-deficient as a pre-adolescent child. PGHD may be caused by damage or trauma to the pituitary gland. The damage is typically caused by a tumor (e.g., a tumor in and/or around the pituitary gland; or a tumor in the hypothalamus). Pituitary tumors can compress the gland or damage can occur when the tumor is removed via neurosurgery. The pituitary can also be damaged by infection, blood vessel disease, severe head injury, or cranial radiation or chemotherapy for treating tumors of the head and neck. PGHD may be caused by: trauma that occurred in a child or adolescent at their birth or soon after their birth; central nervous system infection; tumors of the hypothalamus or pituitary glands; infiltrative or granulomatous disease; cranial irradation; surgery; or idiopathic causes.

2. hGH-XTEN Bolus Doses and Dosage Regimens

In one aspect, the present invention provides a method of treating pediatric growth hormone deficiency (PGHD) in a human pediatric patient by administering a human growth hormone-XTEN (hGH-XTEN) fusion protein to the patient. In one embodiment, the method comprises administering the hGH-XTEN fusion protein to the pediatric patient as a bolus dose. In another embodiment, the bolus dose is a therapeutically effective bodyweight adjusted bolus dose. In one other embodiment, the bolus dose is between about 0.8 mg/kg and about 6.3 mg/kg. In one embodiment, the fusion protein comprises an amino acid sequence having at least about 90% sequence identity to SEQ ID NO:1. In another embodiment, the fusion protein comprises an amino acid sequence having at least about 91%, or at least about 92%, or at least about 93%, or at least about 94%, or at least about 95%, or at least about 96%, or at least about 97%, or at least about 98%, or at least about 99% sequence identity to SEQ ID NO:1. In another embodiment, the fusion protein comprises an amino acid sequence having the sequence of SEQ ID NO:1.

In one aspect, the bolus dose may be administered over a range of doses. It should be noted that where reference is made to the administration of a bolus dose between about a first mg/kg and about a second mg/kg, the “first mg/kg” term may include the first mg/kg value and the “second mg/kg” term may include the second mg/kg value.

In one embodiment, the hGH-XTEN fusion protein comprises (i) an amino acid sequence having at least about 90% sequence identity to SEQ ID NO:1; (ii) the amino acid sequence of SEQ ID NO:1; (iii) an amino acid sequence having at least about 90% sequence identity to SEQ ID NO:4 (AE912-hGH); (iv) the amino acid sequence of SEQ ID NO:4 (AE912-hGH); (v) an amino acid sequence having at least about 90% sequence identity to SEQ ID NO:38; or (vi) the amino acid sequence of SEQ ID NO:38.

In one other aspect, the bolus dose of the hGH-XTEN fusion protein is administered to a human pediatric patient on a regular basis over a suitable time period, which can be finite or indefinite. In one embodiment, the bolus dose is administered every week, every two weeks, every three weeks, or monthly. In other embodiments, the bolus dose is administered once a month, twice a month, three times a month, or four times a month. In another embodiment, the bolus dose is administered about every 7 days, about every 10 days, about every 14 days, about every 21 days, about every 28 days, or about every 30 days. In one embodiment, the bolus dose is administered on a non-daily basis, or is a non-daily bolus dose.

In an additional aspect, the bolus dose of the hGH-XTEN fusion protein is administered to a human pediatric patient at a dose (i) between about 1.0 mg/kg and about 6.3 mg/kg; (ii) between about 1.0 mg/kg and about 1.5 mg/kg; (iii) between about 2.0 mg/kg and about 3 mg/kg, or (iv) between about 4.5 mg/kg and about 5.5 mg/kg, wherein the dose is administered on a monthly, semimonthly, or weekly basis. In one embodiment, the fusion protein is administered at a dose of about 1.0 mg/kg, about 1.05 mg/kg, about 1.10 mg/kg, about 1.15 mg/kg, about 1.20 mg/kg, about 1.25 mg/kg, about 1.30 mg/kg, about 1.35 mg/kg, about 1.40 mg/kg, about 1.45 mg/kg, and about 1.50 mg/kg, wherein the dose is administered on a monthly, semimonthly, or weekly basis. In another embodiment, the fusion protein is administered at a dose of about 2.0 mg/kg, about 2.10 mg/kg, about 2.20 mg/kg, about 2.30 mg/kg, about 2.40 mg/kg, about 2.50 mg/kg, about 2.60 mg/kg, about 2.70 mg/kg, about 2.80 mg/kg, about 2.90 mg/kg, and about 3.0 mg/kg, wherein the dose is administered on a monthly, semimonthly, or weekly basis. In one other embodiment, the fusion protein is administered at a dose of about 4.50 mg/kg, about 4.60 mg/kg, about 4.70 mg/kg, about 4.80 mg/kg, about 4.90 mg/kg, about 5.0 mg/kg, about 5.10 mg/kg, about 5.20 mg/kg, about 5.30 mg/kg, about 5.40 mg/kg, about 5.50 mg/kg, about 6.0 mg/kg, and about 6.3 mg/kg wherein the dose is administered on a monthly, semimonthly, or weekly basis. In preferred embodiments, the fusion protein is administered (i) at a dose of about 1.15 mg/kg on a weekly basis; (ii) at a dose of about 2.5 mg/kg on a semimonthly basis; and/or (iii) at a dose of about 5.0 mg/kg on a monthly basis.

In another embodiment, the fusion protein is administered at a dose of about 0.8 mg/kg, about 0.9 mg/kg, 1.60 mg/kg, about 1.70 mg/kg, about 1.80 mg/kg, about 1.90 mg/kg, about 3.10 mg/kg, about 3.20 mg/kg, about 3.30 mg/kg, about 3.40 mg/kg, about 3.50 mg/kg, about 3.60 mg/kg, about 3.70 mg/kg, about 3.80 mg/kg, about 3.9 mg/kg, about 4.0 mg/kg, about 4.10 mg/kg, about 4.20 mg/kg, about 4.30 mg/kg, about 4.40 mg/kg, about 5.60 mg/kg, about 5.70 mg/kg, about 5.80 mg/kg, and about 5.90 mg/kg, wherein the dose is administered on a monthly, semimonthly, or weekly basis.

In another aspect, additional bolus doses and ranges of bolus doses of the hGH-XTEN fusion protein for a human pediatric patient are suitable. In one embodiment, the bolus dose of hGH-XTEN is

(i) between about 0.8 mg/kg and about 1.2 mg/kg, about 1.2 mg/kg and about 1.8 mg/kg, about 1.8 mg/kg and about 2.7 mg/kg, about 2.7 mg/kg and about 4 mg/kg, about 4 mg/kg and about 6 mg/kg, about 0.8 mg/kg and about 1.8 mg/kg, about 0.8 mg/kg and about 2.7 mg/kg, or about 0.8 mg/kg and about 4 mg/kg;

(ii) between about 1.2 mg/kg and about 1.8 mg/kg, about 1.2 mg/kg and about 2.7 mg/kg, about 1.2 mg/kg and about 4 mg/kg, or about 1.2 mg/kg and about 6.3 mg/kg;

(iii) between about 1.8 mg/kg and about 2.7 mg/kg, about 1.8 mg/kg and about 4 mg/kg, or about 1.8 mg/kg and about 6 mg/kg;

(iv) between about 2.7 mg/kg and about 4 mg/kg, about 2.7 mg/kg and about 6 mg/kg; or

(v) between about 4 mg/kg and about 6 mg/kg.

In another embodiment, the bolus dose of hGH-XTEN is selected from the group consisting of about 0.8 mg/kg, about 1.0 mg/kg, about 1.2 mg/kg, about 1.4 mg/kg, about 1.6 mg/kg, about 1.8 mg/kg, about 2.0 mg/kg, about 2.2 mg/kg, about 2.4 mg/kg, about 2.6 mg/kg, about 2.7 mg/kg, about 2.8 mg/kg, about 3 mg/kg, about 3.2 mg/kg, about 3.4 mg/kg, about 3.6 mg/kg, about 3.8 mg/kg, about 4 mg/kg, about 4.2 mg/kg, about 4.4 mg/kg, about 4.6 mg/kg, about 4.8 mg/kg, about 5 mg/kg, about 5.2 mg/kg, about 5.4 mg/kg, about 5.6 mg/kg, about 5.8 mg/kg, about 6 mg/kg, and about 6.3 mg/kg.

In one embodiment, the method comprises administering to a human pediatric patient with PGHD at least two bolus doses of a human growth hormone hGH-XTEN fusion protein, wherein said administration is separated by: at least about 7 days, at least about 10 days, at least about 14 days, at least about 21 days, at least about 28 days, or at least about 30 days. In one other embodiment, the bolus dose is a therapeutically effective bodyweight adjusted bolus dose (as described herein). In one other embodiment, the administering step comprises administering a pharmaceutical composition comprising an effective amount of hGH-XTEN fusion protein comprising the amino acid sequence set forth in FIG. 1 (SEQ ID NO:1). In another embodiment, the methods described herein comprise the use of a fusion protein having at least about 90%, or at least about 95%, or at least about 96%, or at least about 97%, or at least about 98%, or at least about 99% sequence identity to the sequence as set forth in FIG. 1 (SEQ ID NO:1).

In another embodiment, the administration of bolus doses is separated by: at least about a month, at least about 31 days, at least about 30 days, at least about 29 days, at least about 28 days, at least about 27 days, at least about 26 days, at least about 25 days, at least about 24 days, at least about 23 days, at least about 22 days, at least about 21 days, at least about 20 days, at least about 19 days, at least about 18 days, at least about 17 days, at least about 16 days, at least about 15 days, at least about 14 days, at least about 13 days, at least about 12 days, at least about 11 days, at least about 10 days, at least about 9 days, at least about 8 days, at least about 7 days, at least about 6 days, at least about 5 days, at least about 4 days, at least about 3 days, or at least about 2 days.

In another embodiment, the therapeutically effective bodyweight adjusted bolus doses of hGH-XTEN fusion protein are administered subcutaneously to the human pediatric patient.

In general, a “bolus dose” is a dose administered within a short period of time. In another embodiment, the bolus dose is administered within about 1 to about 30 minutes, about 1 to about 20 minutes, about 1 to about 15 minutes, about 1 to about 10 minutes, or about 1 to about 5 minutes. In one embodiment, the bolus dose is administered within about 1 to about 5 minutes. In one other embodiment, the bolus does is a subcutaneous bolus dose.

The invention provides methods to establish a dose regimen for the hGH-XTEN pharmaceutical compositions of the invention for human pediatric patients. The methods include administration of consecutive doses of a therapeutically effective amount of the hGH-XTEN composition using variable periods of time between doses to determine that interval of dosing sufficient to achieve and/or maintain the desired parameter, blood level or clinical effect; such consecutive doses of a therapeutically effective amount at the effective interval establishes the therapeutically effective dose regimen for the hGH-XTEN for a PGHD condition. Thus, in one aspect, the invention provides an hGH-XTEN composition for use in a treatment regimen that is therapeutically effective for human growth hormone deficiency (PGHD).

In another aspect, the invention provides an hGH-XTEN fusion protein for use in a treatment regimen for human pediatric growth hormone deficiency (PGHD), which regimen comprises administering a hGH-XTEN fusion protein to a human pediatric patient. In one embodiment, the treatment regimen comprises administering a bolus dose (as described herein) of the hGH-XTEN fusion protein to the human pediatric patient at certain time intervals (as described herein). In one additional embodiment, the treatment regimen comprises subcutaneous administration of the bolus dose of hGH-XTEN. In one embodiment, the regimen comprises administering at least two bolus doses (as described herein) of the hGH-XTEN fusion protein to a human pediatric patient separated by an appropriate time interval (as described herein).

In another embodiment, the present invention provides a consecutive dose regimen wherein each bolus dose of the hGH-XTEN is administered every week (or weekly), every two weeks, every three weeks, every four weeks, or monthly.

In one embodiment of the hGH-XTEN composition for use in a treatment regimen, the hGH-XTEN fusion protein comprises the amino acid sequence shown as set forth in FIG. 1 (SEQ ID NO:1). In one embodiment, the therapeutically effective dose treatment regimen comprises the administration of at least two therapeutically effective bodyweight adjusted bolus doses to a pediatric subject, wherein the doses are administered subcutaneously.

3. hGH-XTEN Equivalency to rhGH

In another aspect, the present invention provides methods of treating human growth hormone deficiency (PGHD) in pediatric patients with a therapeutically effective amount of an hGH-XTEN fusion protein as a bolus dose that is equivalent to, or equivalent to less than, an effective amount of a corresponding hGH (not linked to XTEN) administered daily. In one embodiment, the bolus dose of the fusion protein is equivalent to an amount that is less than between about 4.8 μg hGH/kg/day and about 37 μg hGH/kg/day; or less than or equivalent to about 4.8 μg hGH/kg/day, about 7.4 μg hGH/kg/day, about 11.1 μg hGH/kg/day, about 16.7 μg hGH/kg/day, about 24.7 μg hGH/kg/day, or about 37 μg hGH/kg/day. The approximate mean pediatric rhGH daily dose is 40 μg hGH/kg/day to 43 μg hGH/kg/day. In another embodiment, the bolus dose is a therapeutically effective bodyweight adjusted bolus dose of the hGH-XTEN fusion protein.

In one additional aspect, the present invention provides methods of treating human pediatric growth hormone deficiency (PGHD), comprising administering to a human pediatric patient with PGHD an hGH-XTEN fusion protein at a dosage that is below or less than an equivalent daily dose of recombinant hGH (e.g., a recommended daily dose of rhGH).

In another embodiment, the administration of said bolus doses is separated by at least about 7 days, at least about 10 days, at least about 14 days, at least about 21 days, at least about 28 days, at least about 30 days, or at least about a month.

In one embodiment, the bolus dose of the hGH-XTEN is equivalent to an hGH/kg/day dosage that is less than about 43 μg hGH/kg/day. In another embodiment, the bolus dose of the hGH-XTEN is equivalent to an hGH/kg/day dosage that is less than about 40 μg hGH/kg/day. In another embodiment, the dosage of the hGH-XTEN is equivalent to less than about 39 μg hGH/kg/day, about 38 μg hGH/kg/day, about 36 μg hGH/kg/day, about 34 μg hGH/kg/day, about 32 μg hGH/kg/day, about 30 μg hGH/kg/day, about 28 μg hGH/kg/day, about 26 μg hGH/kg/day, about 25 μg hGH/kg/day, about 24 μg hGH/kg/day, about 22 μg hGH/kg/day, about 20 μg hGH/kg/day, about 18 μg hGH/kg/day, about 17 μg hGH/kg/day, about 16 μg hGH/kg/day, about 14 μg hGH/kg/day, about 12 μg hGH/kg/day, about 11 μg hGH/kg/day, about 8 μg hGH/kg/day, about 7 μg hGH/kg/day, about 6 μg hGH/kg/day, about 5 μg hGH/kg/day, about 4 μg hGH/kg/day, or about 2 μg hGH/kg/day.

In one other embodiment, the bolus dose of the hGH-XTEN is the same or less than the cumulative equivalent hGH/kg/day dosages administered over about 7 days, about 14 days, about 21 days, about 28 days, or about 30 days.

In yet another embodiment, the hGH-XTEN fusion protein comprises an amino acid sequence shown as set forth in FIG. 1 (SEQ ID NO:1). In other embodiments, the administration is subcutaneous administration.

In one aspect, the bolus dose of the hGH-XTEN may be administered over a range of doses that are equivalent to less than an hGH/kg/day dosage. It should be noted that where reference is made to a bolus dose that is equivalent to less than an hGH/kg/day dosage that is between about a first μg hGH/kg/day and about a second μg hGH/kg/day, the “first μg hGH/kg/day” term may include the first μg hGH/kg/day value and the “second μg hGH/kg/day” term may include the second μg hGH/kg/day value.

4. hGH-XTEN and IGF-I Levels

The methods of the present invention are advantageous with respect to resulting IGF-I levels in the human pediatric patient following treatment with hGH-XTEN fusion protein. A high level of blood IGF-I is undesirable since high IGF-I is believed to be a risk factor for cancer (Svensson et al. J Clin Endocrin Metab. epub Sep. 26, 2012 as doi:10.1210/jc.2012-2329). IGF-I generation in humans is largely the result of GH signaling and IGF-I is an important mediator for anabolic actions observed during GH therapy (Le Roith et al. (2001). Endocr Rev 22, 53-74). Accordingly, IGF-I is an important pharmacodynamic marker for hGH-XTEN fusion protein bioactivity. In practice, IGF-I responses to GH (e.g., daily rhGH therapy) are interpreted in terms of age- and gender-specific normative data (Vance et al. (1999). N Engl J Med 341, 1206-16; Molitch et al. (2011). J Clin Endocrinol Metab 96, 1587-609). The interpretation is most readily done with the use of IGF-I standard deviation scores (IGF-I SDS). Further, pediatric patients with GH deficiency, as with healthy individuals, have a range of baseline IGF-I values. Accordingly, IGF-I SDS, corrected for baseline at time 0, can be used to examine potential hGH-XTEN fusion protein dose effects on IGF-I responses.

In one aspect, the present invention provides methods of treatment of PGHD in which the human pediatric patient maintains an IGF-I response (e.g., as measured by mean IGF-I SDS) in a normal range after administration of the hGH-XTEN fusion protein. For an IGF-I SDS, a normal range is generally between about −1.5 and about 1.5 but can also be between about −2.0 and about 2.0.

It should be noted that where reference is made to an IGF-I SDS between about a first value (e.g., −2.0) and about a second value (e.g., 2.0), the “first value” may include the first value and the “second value” may include the second value.

In one embodiment, the present invention provides a method of treating pediatric growth hormone deficiency (PGHD) in a human pediatric patient by administering an hGH-XTEN fusion protein to the patient, wherein the human patient has a serum IGF-I standard deviation score (SDS) between about −2.0 and about 2.0 following administration. In one embodiment, the method comprises administering the hGH-XTEN fusion protein to the pediatric patient as a bolus dose (as described herein). In another embodiment, the bolus dose is a therapeutically effective bodyweight adjusted bolus dose. In other embodiments, the pediatric patient has a serum IGF-I SDS of greater than about −2.0, greater than about −1.5, greater than about −1.0, greater than about −0.5, or greater than about 0, greater than about 0.5, greater than about 1.0, greater than about 1.5, greater than about 1.6, greater than about 1.7, greater than about 1.8, or greater than about 1.9 following administration of the hGH-XTEN.

In another embodiment, the bolus dose of the hGH-XTEN is effective to maintain the pediatric patient's serum IGF-I standard deviation score (SDS) (a) between about −2.0 and about 2.0, or (b) between about 0 and about 2.0 for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, or at least about 30 days following administration of the bolus dose.

In another embodiment, administration of multiple consecutive hGH-XTEN bolus doses is effective to maintain the pediatric patient's serum IGF-I standard deviation score (SDS) (a) between about −2.0 and about 2.0, or (b) between about 0 and about 2.0 for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, or at least about 30 days between administrations of the bolus doses. In the foregoing embodiment, the bolus doses are administered weekly, every two weeks, every three weeks, or monthly.

In another embodiment, administration of multiple consecutive hGH-XTEN bolus doses is effective to maintain the pediatric patient's mean serum IGF-I standard deviation score (SDS) (a) between about −2.0 and about 2.0, or (b) between about −1.0 and about 2.0 for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, or at least about 30 days between administrations of the bolus doses. In the foregoing embodiment, the bolus doses are administered weekly, every two weeks, every three weeks, or monthly.

In another embodiment, administration of multiple consecutive hGH-XTEN bolus doses is effective to maintain the pediatric patient's serum IGF-I standard deviation score (SDS) (a) above about −2.0, or (b) above about 0, or (c) above about 1.0, or (d) above about 1.5 for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, or at least about 30 days between administrations of the bolus doses. In the foregoing embodiment, the bolus doses are administered weekly, every two weeks, every three weeks, or monthly.

In another embodiment, administration of multiple consecutive hGH-XTEN bolus doses is effective to maintain the pediatric patient's serum IGF-I standard deviation score (SDS) (a) below about 1.5, or (b) below about 2.0 for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, or at least about 30 days between administrations of the bolus doses. In the foregoing embodiment, the bolus doses are administered weekly, every two weeks, every three weeks, or monthly.

In another embodiment, administration of multiple consecutive hGH-XTEN bolus doses is effective to maintain the pediatric patient's change in mean maximum serum IGF-I standard deviation score (SDS) compared to baseline SDS (a) between about 0.5 and 3.0, or (b) between about 1.0 and 2.5 for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, or at least about 30 days between administrations of the bolus doses. In the foregoing embodiment, the bolus doses are administered weekly, every two weeks, every three weeks, or monthly.

In another embodiment, the administering step comprises administering a pharmaceutical composition comprising an effective amount of hGH-XTEN fusion protein comprising the amino acid sequence set forth in FIG. 1 (SEQ ID NO:1).

In one other aspect, the present invention provides methods of treating pediatric patients by administering an hGH-XTEN fusion protein to provide a normal serum IGF-I level in the pediatric patient. In one embodiment, the hGH-XTEN fusion protein is administered as a bolus dose (as described herein). In another embodiment, at least two bolus doses are administered separated by a time interval (as described herein). In one other embodiment, the bolus dose(s) is a therapeutically effective bodyweight adjusted bolus dose of the fusion protein. In an additional other embodiment, the administration of said bolus dose(s) of the hGH-XTEN results in a normalization of serum IGF-I levels in the a pediatric subject for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, or at least about a month following administration of the bolus dose. In one other embodiment, a normal serum IGF-I level is characterized by a serum IGF-I standard deviation (SD) that is above about −2.0; above about −1.5; above about −1.0; above about 0; above about 0.5; above about 1.0; or above about 1.5. In another embodiment, a normal serum IGF-I level is characterized by a serum IGF-I standard deviation (SD) that is between about −1.5 and about 1.5; between about −1.5 and about 1.0; between about −1.5 and about 0.5; between about −1.5 and about 0; between about −1.5 and about −0.5; and between about −1.5 and about −1.0.

In an additional embodiment, the extent of normalization of IGF-I serum levels is dependent on the dose of the therapeutically effective bodyweight adjusted bolus dose of hGH fusion protein. In one other embodiment, the duration of the IGF-I normalization increases with the therapeutically effective bodyweight adjusted bolus dose of hGH fusion protein.

The methods of the present invention provides a particular advantage in that that the administration of hGH-XTEN fusion protein provides an observable and prolonged IGF-I response in the human pediatric patient (e.g., as measured by IGF-I SDS) that is not accompanied by, or at the expense of, over-exposure to high levels of IGF-I, which is undesirable. In other words, the IGF-I response is maintained at an elevated level that is still considered acceptable by current standards, e.g., as indicated by an IGF-I SDS of 1.5 or less, or an IGF-I SDS of 2.0 or less.

5. Plasma Concentration of hGH-XTEN Fusion Protein

In another aspect, the invention provides a method of treating human pediatric growth hormone deficiency (PGHD) in a human pediatric patient by administering an hGH-XTEN fusion protein to the patient, wherein the patient has a plasma concentration of said fusion protein of at least about 10 ng/mL following administration. In one embodiment, the method comprises administering the hGH-XTEN fusion protein to the pediatric patient as a bolus dose (as described herein). In another embodiment, the bolus dose of the hGH-XTEN is a therapeutically effective bodyweight adjusted bolus dose (as described herein). In one embodiment, the bolus dose is selected from the group consisting of about 0.8 mg/kg, about 1.0 mg/kg, about 1.2 mg/kg, about 1.4 mg/kg, about 1.6 mg/kg, about 1.8 mg/kg, about 2.0 mg/kg, about 2.2 mg/kg, about 2.4 mg/kg, about 2.6 mg/kg, about 2.7 mg/kg, about 2.8 mg/kg, about 3 mg/kg, about 3.2 mg/kg, about 3.4 mg/kg, about 3.6 mg/kg, about 3.8 mg/kg, about 4.0 mg/kg, about 4.2 mg/kg, about 4.4 mg/kg, about 4.6 mg/kg, about 4.8 mg/kg, about 5.0 mg/kg, about 5.2 mg/kg, about 5.4 mg/kg, about 5.6 mg/kg, about 5.8 mg/kg, about 6.0 mg/kg, and about 6.3 mg/kg. In another embodiment, the bolus dose of the hGH-XTEN is effective to maintain a plasma concentration of the fusion protein of at least about 10 ng/mL for: at least about 5 days, at least about 7 days, at least about 10 days, at least about 14 days, at least about 20 days, at least about 25 days, at least about 30 days, or at least about a month. In another embodiment, the bolus dose is effective to maintain a plasma concentration of the fusion protein of at least about 100 ng/mL for: at least about 5 days, at least about 7 days, at least about 10 days, at least about 14 days, or at least about 20 days. In one other embodiment, the administering step comprises administering a pharmaceutical composition comprising an effective amount of hGH-XTEN fusion protein comprising the amino acid sequence set forth in FIG. 1 (SEQ ID NO:1).

6. Absence of Side Effects

In one embodiment, the invention provides a method of treating human pediatric growth hormone deficiency (PGHD) in a human pediatric patient comprising administering to the patient an hGH-XTEN fusion protein in the absence of one or more side effects. In one other embodiment, the absence of one or more side effects is the absence of a clinically significant level of one or more side effects. In another embodiment, the one or more side effects that are absent are selected from the group consisting of headache, arthalgia, myalgia, edema, nausea, and muscle fatigue after administration of the fusion protein. As used herein, “clinically significant level of a side-effect” means that the side-effect(s) is/are not unexpected or is/are not serious adverse event(s). Side-effects that are mild and transient, even if one of headache, arthalgia, myalgia, edema, nausea, and muscle fatigue or those otherwise known to be associated with the administration of growth hormone, would not be considered a clinically significant level. In one embodiment, the method comprises administering the hGH-XTEN fusion protein to the pediatric patient as a bolus dose (as described herein). In another embodiment, the bolus dose of the hGH-XTEN fusion protein is a therapeutically effective bodyweight adjusted bolus dose (as described herein). In one other embodiment, the bolus dose is administered subcutaneously. In one other embodiment, the administering step comprises administering a pharmaceutical composition comprising an effective amount of hGH-XTEN fusion protein comprising the amino acid sequence set forth in FIG. 1 (SEQ ID NO:1).

7. Parameters Following Administration

In one embodiment, the invention provides a method for achieving a beneficial effect in a human pediatric patient with growth hormone deficiency, comprising the step of administering to the pediatric patient a therapeutically-effective amount of a hGH-XTEN fusion protein wherein said administration results in the improvement of one or more biochemical or physiological parameters or clinical endpoints associated with a growth hormone-related disease, disorder or condition, including a PGHD (as described herein). The effective amount produces a beneficial effect in helping to treat (e.g., cure or reduce the severity) the deleterious effects of a growth hormone-related disease, disorder or condition. In some cases, the method for achieving a beneficial effect includes administering a therapeutically effective amount of a hGH-XTEN fusion protein composition to treat a pediatric patient with a growth hormone-related disease, disorder, or condition, including a PGHD (as described herein).

The methods of the invention include the administration to a human pediatric patient of successive or consecutive doses of a therapeutically effective amount of the hGH-XTEN for a period of time sufficient to achieve and/or maintain the desired parameter or clinical effect, and such consecutive doses of a therapeutically effective amount establishes the therapeutically effective dose regimen for the hGH-XTEN; i.e., the schedule for consecutively administered doses of the fusion protein composition, wherein the doses are given in therapeutically effective amounts to result in a sustained beneficial effect on any clinical sign or symptom, aspect, measured parameter or characteristic of a metabolic disease state or condition, including, but not limited to, those described herein. In one embodiment of the method, the parameters include but are not limited to mean (SD) height standard deviation score (HT-SDS), changes in height velocity, IGF-I concentration, ratio of IGF-I/IGFBP-3, IGFBP3 concentration, change in weight, lean body mass, change in body mass index, total body fat (adipose fat/tissue), trunk fat, response to insulin challenge, rate of division of chondrocytes, chondrocyte numbers, bone density, bone age, bone growth, bone turnover, increase in epiphyseal plate width, reduction in cholesterol, reduction in triglycerides, and reduction in LDL. In another embodiment of the method, the administration to a human pediatric patient of successive or consecutive doses of a therapeutically effective amount of the hGH-XTEN results in a beneficial effect in two or more of the parameters including, but not limited to mean (SD) height standard deviation score (HT-SDS), changes in height velocity, IGF-I concentration, ratio of IGF-I/IGFBP-3, IGFBP3 concentration, change in weight, lean body mass, change in body mass index, total body fat (adipose fat/tissue), trunk fat, response to insulin challenge, rate of division of chondrocytes, chondrocyte numbers, bone density, bone age, bone growth, bone turnover, increase in epiphyseal plate width, reduction in cholesterol, reduction in triglycerides, and reduction in LDL.

Height velocity data in pediatric patients treated with recombinant human growth hormone (rhGH) has been compiled into various databases. The National Cooperative Growth Study (NCGS) database contains 220,000 patient-years of growth data on children receiving rhGH therapy. The NCGS database was initiated in December 1985 to collect data in children treated with rhGH for evaluation of safety and efficacy. Anonymous data were entered by clinical investigators in the US including date of birth, sex, height, weight, etiology of short stature, peak serum GH response to stimulation testing, Tanner pubertal stages, parental heights, and GH dose for patients treated with Genentech's rhGH products (Shulman, D I, et al. Int J Pediatr Endocrinol. 2013; 2013(1): 2). It has been shown that height velocity observed during the first year of treatment with GH is the major determinant of the second pre-pubertal year growth response to GH in small for gestational age (SGA) children (Ranke M B, et al. J Clin Endocrinol Metab. 2003; 88:125-131). The first year height velocity can be measured in the pediatric patient over a period of 3 months, 4 months, 6 months, or other period up to 12 months to ascertain the annualized first year height velocity, expressed as “cm/yr”.

In other embodiments of the method for achieving a beneficial effect in a human pediatric patient with growth hormone deficiency, the methods comprise the step of administering to the pediatric patient a therapeutically-effective amount of a hGH-XTEN fusion protein wherein said administration results in the improvement in height velocity rate in the pediatric patient. In one embodiment of the method, the method is effective to achieve a height velocity equivalent to 7 cm/yr to 12 cm/yr in a pediatric patient. In another embodiment of the method, the method is effective to achieve a height velocity equivalent to 8 cm/yr to 11 cm/yr in a pediatric patient. In one embodiment, the height velocity is achieved (or determined) after at least 3 months, or at least 6 months, or at least 12 months of dosing in the pediatric patient. In another embodiment, the height velocity achieved is a first year height velocity. In another embodiment, the method is not inferior to the height velocity achieved with daily injections of hGH not linked to XTEN over the same period and administered using comparable equivalent doses on a molar basis. In another embodiment, the method is effective to maintain the pediatric patient's annualized height velocity after at least 3 months of dosing within 10%, 20%, or 30% of that compared to the height velocity achieved with daily injections of an hGH not linked to XTEN of an equivalent amount, on a molar basis, over the same period. In one embodiment of the foregoing, the pediatric patients administered daily injections of hGH not linked to XTEN receive a dose of at least about 25, at least about 30, at least about 33, at least about 35 μg rhGH/kg/day, at least at least about 37 μg rhGH/kg/day, or at least about 43 μg rhGH/kg/day. In the foregoing embodiments of this paragraph, the bolus dose of the hGH-XTEN fusion protein is a therapeutically effective bodyweight adjusted bolus dose comprising between about 0.8 mg/kg and about 6.3 mg/kg of hGH-XTEN fusion protein. In another embodiment, the bolus dose of the hGH-XTEN fusion protein is a therapeutically effective bodyweight adjusted bolus dose comprising between about 0.8 mg/kg and about 7.0 mg/kg of hGH-XTEN fusion protein. In another embodiment, the bolus doses are administered every week, every two weeks, every three weeks, semimonthly or monthly. In another embodiment, the pediatric patients are administered bolus doses of about 1.15 mg/kg of hGH-XTEN fusion protein weekly, or about 2.5 mg/kg of hGH-XTEN fusion protein every two weeks, or about 5.0 mg/kg of hGH-XTEN fusion protein monthly. In another embodiment, the pediatric patients are administered bolus doses selected from about 0.8 mg/kg to about 1.5 mg/kg, about 1.8 mg/kg to about 3.2 mg/kg, or about 3.5 mg/kg to about 6.3 mg/kg. In a preferred embodiment, the pediatric patients are administered bolus doses of at least about 5.0 mg/kg of hGH-XTEN fusion protein monthly.

In another embodiment of the regimen, the human pediatric patient achieves an improvement after two or more bolus doses in at least one parameter selected from bone density, bone growth, and increase in epiphyseal plate width. In one other embodiment, the foregoing improvement(s) is at least about 10%, or at least about 20%, or at least about 30%, or at least about 40%, or at least about 50%, or at least about 60%, or at least about 70%, or at least about 80%, or at least about 90% compared to a human pediatric patient not receiving human growth hormone. In another embodiment, the foregoing percentage improvement(s) is similar to, or not inferior to, an improvement achieved by an hGH not linked to XTEN and administered daily using daily dosage equivalent amounts of hGH.

8. hGH-XTEN Medicaments

In another embodiment, the present invention provides an hGH-XTEN fusion protein for use as a medicament, or for the treatment of PGHD in pediatric patients. In another embodiment, the present invention provides the use of an hGH-XTEN fusion protein for the manufacture of a medicament for treating PGHD in a human pediatric patient with PGHD. In one other embodiment, the present invention provides the use of the fusion protein having the sequence set forth in FIG. 1 (SEQ ID NO:1) in the manufacture of a medicament for the treatment of PGHD in pediatric patients. In other embodiments, the hGH-XTEN fusion protein is provided as a bolus dose (as described herein). In another embodiment, the bolus dose is a therapeutically effective bodyweight adjusted dose. In another embodiment, the medicament is formulated for subcutaneous administration. In one other embodiment, the hGH-XTEN fusion protein comprises an amino acid sequence shown as set forth in FIG. 1 (SEQ ID NO:1).

9. Treatment of Indicia of Pediatric GH-Related Conditions

In another aspect, the present invention provides hGH-XTEN fusion protein-based therapeutic agents for treating diseases or conditions related to pediatric growth hormone deficiency (PGHD) in a pediatric patient. For the prevention, treatment or reduction in the severity of a given disease or condition, the appropriate dosage of a therapeutic agent of the invention will depend on the type of disease or condition to be treated, as defined above, the severity and course of the disease or condition, whether the agent is administered for therapeutic purposes, previous therapy, the pediatric patient's clinical history and response to the agent, and the discretion of the attending physician.

In another aspect, the present invention provides a method for the delaying or slowing down of the progression of a disease or condition related to PGHD in a pediatric patient. In one embodiment, the method comprises administering to pediatric subject diagnosed with the disease, condition, or disorder, an effective amount of an hGH-XTEN fusion protein. In another aspect, the invention provides a method for treating or ameliorating indicia of a disease or condition related to PGHD. In one embodiment, the method comprises administering an effective amount of an hGH-XTEN fusion protein to a pediatric subject at risk of the disease or condition, wherein the hGH-XTEN fusion protein is effective against the development of indicia of the disease or condition.

In one additional aspect, the hGH-XTEN fusion proteins provide an ameliorative effect against the development of, or the progression of, clinical and/or histological and/or biochemical and/or pathological indicia (including both symptoms and signs) of diseases or conditions related to PGHD in a human pediatric patient. In one embodiment, the disease or condition is PGHD. In one embodiment, the indicia in pediatric patients include small stature, an increased level of body fat (especially central or trunk adiposity, i.e, the waist), slow rate of growth of all body parts, leveling off or falling away from an established growth curve for height, delayed bone age, decreased IGF-I SDS, and below average height SDS. In another embodiment, the pediatric subject is at risk for a disease of condition related to PGHD. In general, a pediatric subject at risk will previously have incurred some damage to the pituitary gland and/or the hypothalamus. In one embodiment, the pediatric subject at risk was previously diagnosed as having a tumor associated with the pituitary gland, and/or underwent surgery, chemotherapy, or radiation therapy to treat the tumor. In another embodiment, the pediatric subject at risk previously had or presently has a reduced blood supply to the pituitary gland. In one other embodiment, the pediatric subject at risk previously suffered cranial ablation or has a history of head trauma. In some embodiments, the pediatric subject at risk previously or presently suffers from a hypothalamic-pituitary disease or disorder.

The efficacy of the treatment of diseases and conditions described herein (including PGHD) can be measured by various assessments commonly used in evaluating PGHD in pediatric patients. For example, the health of hormone-secreting glands can be evaluated by, but not limited to, e.g., IGF-I standard deviation score (SDS), mean (SD) height standard deviation score (HT-SDS), growth hormone stimulation test (GHST), growth hormone releasing hormone (GHRH), stimulation tests, monitoring or measurement of endogenous hHG pulses, IGF-I levels, IGF-I binding protein levels, other blood or biochemical tests (e.g., total cholesterol, low-density lipoprotein (LDL) cholesterol, high-density lipoprotein (HDL) cholesterol, triglyceride, and lipids).

In one additional aspect, the present invention provides methods of increasing the efficacy of human growth hormone (hGH) therapy in a human pediatric patient. In another aspect, the present invention provides methods of determining a subsequent dose of an hGH-XTEN fusion protein administered over a subsequent dosage period when treating a human pediatric patient with PGHD with the hGH-XTEN fusion protein. The “dosage period” means the time between the administration of a bolus dose (e.g., initial dose) and the next successive administration of a bolus dose (e.g., subsequent dose). The dosage period may change with one or more further successive dose or doses, or may remain constant.

In one embodiment, the foregoing methods of increasing efficacy comprise the step of monitoring the IGF-I standard deviation score (SDS) in a plasma or serum sample obtained from the pediatric patient during an initial dosage period of administration of an initial dose of human growth hormone-XTEN (hGH-XTEN) fusion protein. In one embodiment, the hGH-XTEN fusion protein comprising an amino acid sequence having at least about 90% sequence identity to SEQ ID NO:1. In another embodiment, the method further comprises the step of determining a subsequent dose of hGH-XTEN fusion protein administered over a subsequent dosage period based on the IGF-I SDS observed during the initial dosage period. In one additional embodiment, the method further comprises administering the subsequent dose over a subsequent dosage period. In one other embodiment, the subsequent dose improves the efficacy of the treatment during the subsequent dosage period. In another embodiment, the subsequent dose is higher, lower, or equivalent to the initial dose. The initial dose or subsequent dose may be any of the bolus doses described herein. In one additional embodiment, the subsequent dosage period is longer, shorter, or equivalent to the initial dosage period. The initial dosage period or subsequent dosage period may be any of the periods of time described herein (e.g., weekly, every two weeks, semimonthly, every three weeks, monthly, etc., or every 7 days, every 10 days, every 14 days, every 21 days, every 30 days, etc.).

### VII). Dosage Forms and Pharmaceutical Compositions

In another aspect, the present invention provides bolus doses or dosage forms comprising an hGH-XTEN fusion protein described herein.

In one embodiment, the bolus dose or dosage of an hGH-XTEN fusion protein comprises a therapeutically effective bodyweight adjusted bolus dose for a human pediatric patient. In one other embodiment, the bolus dose or dosage comprises between about 0.8 mg/kg and about 6.3 mg/kg of hGH-XTEN fusion protein. Other bolus doses are described herein.

In other embodiments, the bolus dose or dosage is (i) for use in treating human PGHD in a pediatric subject in need; and/or (ii) formulated for subcutaneous administration. In one other embodiment, the hGH-XTEN fusion protein comprises the amino acid sequence shown as set forth in FIG. 1 (SEQ ID NO:1). In one embodiment, the bolus dose or dosage form is a pharmaceutical composition comprising the fusion protein having the sequence as set forth in FIG. 1 (SEQ ID NO:1) and a pharmaceutically acceptable carrier.

In another embodiment, the invention provides kits, comprising packaging material and at least a first container comprising the pharmaceutical composition of the foregoing embodiment and a label identifying the pharmaceutical composition and storage and handling conditions, and a sheet of instructions for the preparation and/or administration of the pharmaceutical compositions to a pediatric subject.

In one additional aspect, the present invention provides compositions, pharmaceutical compositions, and dose amounts of an hGH-XTEN fusion protein. In one other embodiment, the pharmaceutical composition or dose amount comprises a fusion protein having the sequence as set forth in FIG. 1 (SEQ ID NO:1), or a sequence having at least about 90%, at least about 91%, or at least about 92%, or at least about 93%, or at least about 94%, or at least about 95%, or at least about 96%, or at least about 97%, or at least about 98%, or at least about 99%, sequence identity to the sequence of SEQ ID NO:1. In another embodiment, the dose amount is for a human pediatric patient based upon the weight of the patient. The weight of the pediatric human patient can range from about 10 kg to about 50 kg. In one additional embodiment, the hGH-XTEN fusion protein is provided in the pharmaceutical composition, composition, or dose amount as a certain quantity. In one other embodiment, the pharmaceutical composition or dose amount further comprises a pharmaceutically acceptable carrier.

In one embodiment, the pharmaceutical composition is administered at a therapeutically effective dose. In another embodiment, the pharmaceutical composition is administered using multiple consecutive doses using a therapeutically effective dose regimen (as defined herein) for the length of the dosing period.

A therapeutically effective amount of the hGH-XTEN varies according to factors such as the disease state, age, sex, and weight of the individual, and the ability of the fusion protein to elicit a desired response in the individual. A therapeutically effective amount is also one in which any toxic or detrimental effects of the hGH-XTEN are outweighed by the therapeutically beneficial effects.

It should be noted that where reference is made to a composition, pharmaceutical composition or dose amount comprising an amount of hGH-XTEN fusion protein between about a first mg and about a second mg, the “first mg” term may include the first mg value and the “second mg” term may include the second mg value.

In another aspect, the present invention provides hGH-XTEN fusion proteins for use in a pharmaceutical regimen or therapeutically effective dose regimen for the treatment of PGHD. In one embodiment, the hGH-XTEN fusion protein is for use in a regimen comprising a bolus dose of the fusion protein to treat a pediatric patient. In an additional embodiment, the regimen comprises the step of determining the amount of the hGH-XTEN fusion protein needed to achieve an IGF-I standard deviation score (SDS) between about −2.0 and about 2.0 in the pediatric patient.

In one other embodiment, the regimen comprises a therapeutically effective bodyweight adjusted bolus dose. In another embodiment, the regimen comprises a bolus dose of the fusion protein that is between about 0.8 mg/kg and about 6.3 mg/kg. In one other embodiment, the regimen comprises the administration of consecutive bolus doses of fusion protein. In one embodiment, the administration of consecutive bolus doses is about every week, about every two weeks, about every three weeks, or about every month. In one additional embodiment, the fusion protein comprises an amino acid sequence having at least about 90% sequence identity to SEQ ID NO:1. In one embodiment, the regimen comprises subcutaneous administration of the bolus dose of the fusion protein. In another embodiment, the regimen is effective to treat PGHD in a pediatric patient.

### VIII). Articles of Manufacture

In one aspect, the present invention also provides kits and articles of manufacture containing materials useful for the treatment, prevention and/or diagnosis of disease (e.g., PGHD) in pediatric patients. In another embodiment, the invention provides kits, comprising packaging material and at least a first container comprising a dosage form or pharmaceutical composition of the foregoing embodiment and a label identifying the dosage form or pharmaceutical composition and storage and handling conditions, and a sheet of instructions for the reconstitution and/or administration of the dosage form or pharmaceutical compositions to a pediatric subject. In one other embodiment, the kit includes a container and a label, which can be located on the container or associated with the container. The container may be a bottle, vial, syringe, cartridge (including autoinjector cartridges), or any other suitable container, and may be formed from various materials, such as glass or plastic. The container holds a composition having an hGH-XTEN fusion protein as described herein, and may have a sterile access port. Examples of containers include a vial with a stopper that can be pierced by a hypodermic injection needle. The kits may have additional containers that hold various reagents, e.g., diluents, preservatives, and buffers. The label may provide a description of the composition as well as instructions for the intended use in pediatric patients.

In one other aspect, the container is a pre-filled syringe. In one embodiment, the syringe is pre-filled with a composition having an hGH-XTEN fusion protein as described herein. In one additional aspect, the present invention provides containers of the composition having a hGH-XTEN fusion protein as described herein, wherein the container is suitable for autoinjection of the composition. In one embodiment, the container is a cartridge. In another embodiment, the container is a cartridge in an autoinjection pen. Those of ordinary skill in the art will appreciate that other suitable autoinjection devices may be used for the present invention. In some embodiments, the autoinjection device comprises a spring-loaded syringe within a cylindrical housing that shields the needle tip prior to injection. In one embodiment, the pediatric patient depresses a button on the device and the syringe needle is automatically inserted to deliver the contents.

In another embodiment, the device is a gas jet autoinjection device. In other embodiments, the gas jet device comprises a cylinder of pressurized gas but the needle is absent. Upon activation, the device propels a fine jet of liquid through the skin without the use of a needle. In one other embodiment, the device is an iontophoresis device or electromotive drug administration (EMDA) device (e.g., use of a small electric charge to deliver an agent through the skin without the use of a needle).

The kit has at least one container that includes a composition comprising an hGH-XTEN fusion protein described herein as the active agent. The container may comprise an hGH-XTEN fusion protein dosage form or a pharmaceutical composition. A label may be provided indicating that the dosage form or composition may be used to treat a disease in a pediatric patient. The label may also provide instructions for administration to a pediatric subject in need of treatment. The kit may further contain an additional container having a pharmaceutically-acceptable buffer, such as bacteriostatic water for injection (BWFI), phosphate-buffered saline, Ringer's solution and dextrose solution. Finally, the kit may also contain any other suitable materials, including other buffers, diluents, filters, needles, and syringes.

In one aspect, the present invention provides a kit comprising a container which holds a pharmaceutical composition for administration to a human pediatric patient comprising a human growth hormone-XTEN (hGH-XTEN) fusion protein. In one embodiment, the hGH-XTEN fusion protein comprises an amino acid sequence having at least about 90% sequence identity to the sequence set forth in FIG. 1 (SEQ ID NO:1). In another embodiment, the kit further comprises a package insert associated with said container. In one other embodiment, the package insert indicates that said composition is for the treatment of growth hormone deficiency by administration of more than one dose of the composition. In one embodiment, the administration is an administration of an initial dose of between about 0.8 mg/kg and about 6.3 mg/kg of the hGH-XTEN and a plurality of subsequent doses of the hGH-XTEN in an amount of between about 0.8 mg/kg and about 6.3 mg/kg. In another embodiment, the doses are separated in time from each other by at least about 7 days. The package insert may further indicate different doses, dose ranges, and times between doses as described herein.

The following are examples of methods, treatment regimens, and compositions of the invention. It is understood that various other embodiments may be practiced, given the general description provided above.

### EXAMPLES

### Example 1A

### Single Dose Results

A Phase 1b/2a trial of the safety, the pharmacokinetics (PK) and pharmacodynamics (PD) of a single dose of a human growth hormone analogue (a human growth hormone-XTEN (hGH-XTEN) fusion protein shown as SEQ ID NO:1, FIG. 1) for subcutaneous (SC) administration in pediatric patients with growth hormone deficiency was conducted. Based on the safety profile in GHD adults (Yuen, K. C. et al. The Journal of Clinical Endocrinology and Metabolism 98, 2595-2603 (2013) and the potential to achieve once-monthly dosing, the Phase 1b/2a study in GHD children determined (i) the safety, tolerability, PK, and IGF-I responses to a single dose of the hGH-XTEN fusion protein in GHD children (Phase 1b); and (ii) the 6 month height velocity (Phase 2a) using fusion protein dosing regimens that normalize IGF-I.

The study was designed to enroll up to 72 naïve-to-treatment, pre-pubertal children. Key inclusion criteria are pre-pubertal status, short stature (HT-SDS≦−2.00), GHD diagnosed by paired GH stimulation tests (GH max≦10 ng/mL), an IGF-I standard deviation score (IGF-I SDS)≦−1 and an absence of other illness or medication use that could impair data interpretation. In Phase 1b, the PK and PD (IGF-I and IGFBP-3) responses over a 30 day period were determined following single subcutaneous doses of up to 6 ascending dosing levels of the hGH-XTEN fusion protein (SAD design). Safety was reviewed before each dose escalation including collected data against protocol-specified stopping criteria. hGH-XTEN fusion protein dose selection for Phase 2a was based on safety and IGF-I responses. Following dose selection, subjects were randomized to a maximum of three cohorts of different doses and/or dose regimens for the determination of 6-month height velocities after repeat dosing. hGH-XTEN fusion protein dosing began at 0.80 mg/kg, a safe and well tolerated dose in GHD adults, with increases to 1.20 mg/kg, 1.80 mg/kg, 2.70 mg/kg, 4.00 mg/kg and up to 6.00 mg/kg.

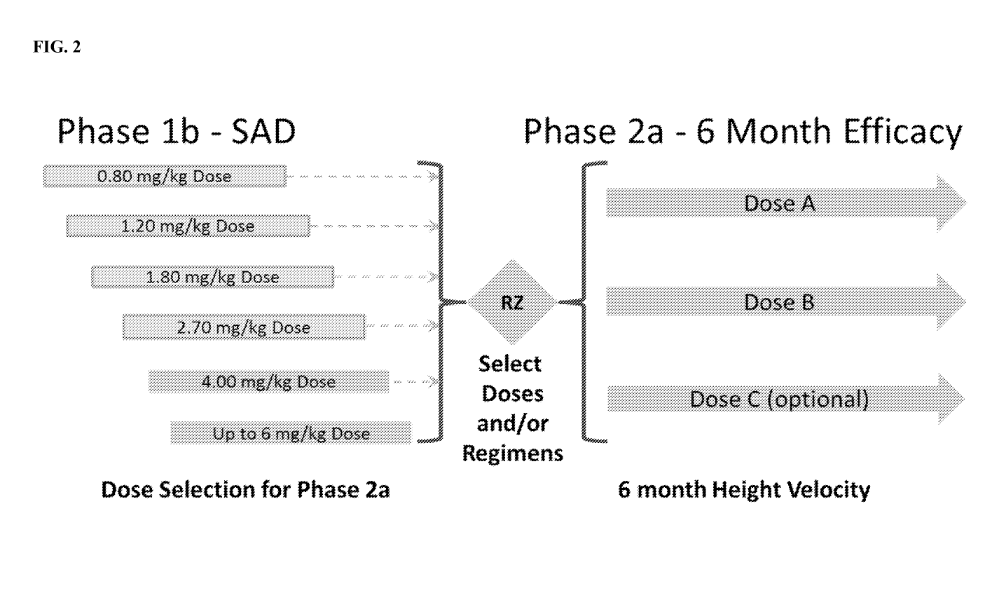

FIG. 2 summarizes the design for the Phase 1b/2a study. Table 1.1 provides the Phase 1b dose levels. hGH-XTEN fusion protein dose levels are below the mean pediatric GHD daily recombinant human growth hormone (rhGH) dose of 40 μg/kg/day administered over 30 days. Doses were selected for Phase 2a based on potential to normalize IGF-1 exposure for a 30 day period.

TABLE 1.1
Phase 1b Dose Levels

| Dose Level | hGH-XTEN fusion protein Dose | rhGH equivalent |
|---|---|---|
| Group | (mg/kg - one dose) | (μg/kg/day × 30 days) |
| 1 | 0.80 | 4.8 |
| 2 | 1.20 | 7.4 |
| 3 | 1.80 | 11.1 |
| 4 | 2.70 | 16.7 |
| 5 | 4.00 | 24.7 |
| 6 | Up to 6.00 | Up to 37.0 |

Table 1.2 shows the Clinical Characteristics of Completed Dosing Groups; Numerical values are means (SD).

TABLE 1.2

| | hGH-XTEN fusion protein Dose | | | |
|---|---|---|---|---|
| | 0.8 mg/kg | 1.2 mg/kg | 1.8 mg/kg | 2.7 mg/kg |
| # Subjects | 8 | 8 | 8 | 8 |
| Age | 7.1 (1.8) | 7.0 (2.2) | 7.6 (2.1) | 7.6 (2.7) |
| Gender (M/F) | 3/5 | 5/3 | 7/1 | 6/2 |
| Height-SDS | −2.6 (0.8) | −2.8 (0.7) | −2.8 (0.4) | −2.6 (0.3) |
| BMI (kg/m²) | 15.4 (1.4) | 15.4 (1.3) | 16.1 (1.8) | 15.3 (1.1) |
| IGF-I SDS | −1.8 (0.7) | −1.7 (0.6) | −1.8 (0.8) | −1.6 (0.3) |
| Screening | | | | |
| IGF-I SDS | −1.4 (0.8) | −2.0 (0.6) | −1.7 (0.9X) | −1.4 (0.5) |
| Baseline | | | | |
| GHₘₐₓ | 5.4 (3.6) | 4.8 (2.2) | 5.6 (3.3) | 6.8 (2.0) |
| (ng/mL) | | | | |
| (stim test) | | | | |

Phase 1b dosing and data collection are complete for the 0.80, 1.20, 1.80 and 2.70 mg/kg hGH-XTEN fusion protein groups. Data are complete for doses ranging from 0.80 to 2.7 mg/kg (equivalent to 4.8 to 16.7 μg rhGH/kg taken daily for 30 days).

The data support that the hGH-XTEN fusion protein is safe and well tolerated. Table 1.3 shows adverse events (AEs) considered as possibly, probably or definitely related to study drug in dose level groups 1-4. All related AEs are CTCAE Grade 1 (mild). There were no SAEs, no unexpected AEs, no patient withdrawals, and no lipoatrophy in any of the enrolled children. The events were judged to be typical of those observed when rhGH treatment is begun in naïve to treatment children with GHD.

TABLE 1.3

| | hGH-XTEN fusion protein Dose | | | |
|---|---|---|---|---|
| | 0.80 mg/kg | 1.20 mg/kg | 1.80 mg/kg | 2.70 mg/kg |
| Event | (n = 8) | (n = 8) | (n = 8) | (n = 8) |
| # Subjects, | 1 | 3 | 3 | 5 |
| any AE | | | | |
| Injection Site | 1 | 1 | 3 | 3 |
| Discomfort | | | | |
| Erythema at | 0 | 1 | 0 | 0 |
| injection site | | | | |
| Headache | 0 | 0 | 1 | 1 |
| Dizziness | 0 | 0 | 1 | 0 |
| Malaise | 0 | 0 | 1 | 0 |
| Myalgia | 0 | 0 | 0 | 1 |
| Arthralgia | 0 | 0 | 0 | 1 |
| Sore Feet | 0 | 0 | 0 | 1 |
| Increased | 0 | 0 | 0 | 1 |
| Hunger | | | | |
| Pruritic | 0 | 1 | 0 | 0 |
| Rash | | | | |

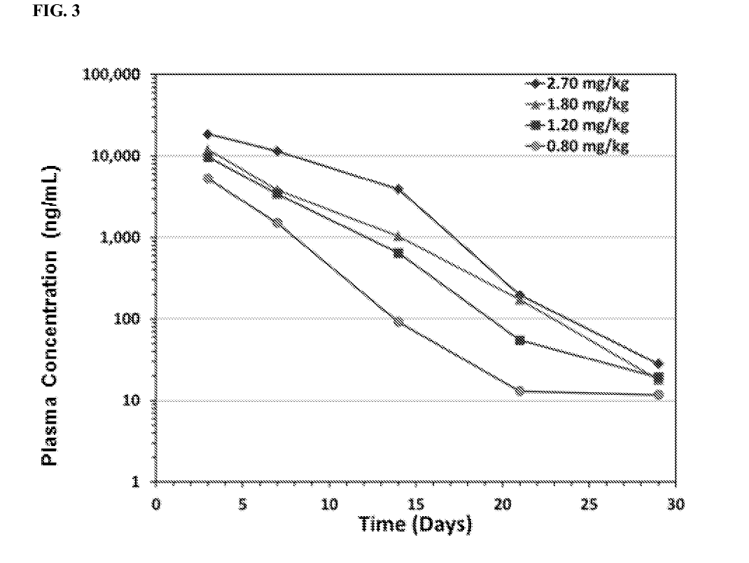

hGH-XTEN fusion protein plasma levels were sustained up to 30 days after a single dose. FIG. 3 shows the hGH-XTEN fusion protein plasma concentration (ng/mL) mean values.

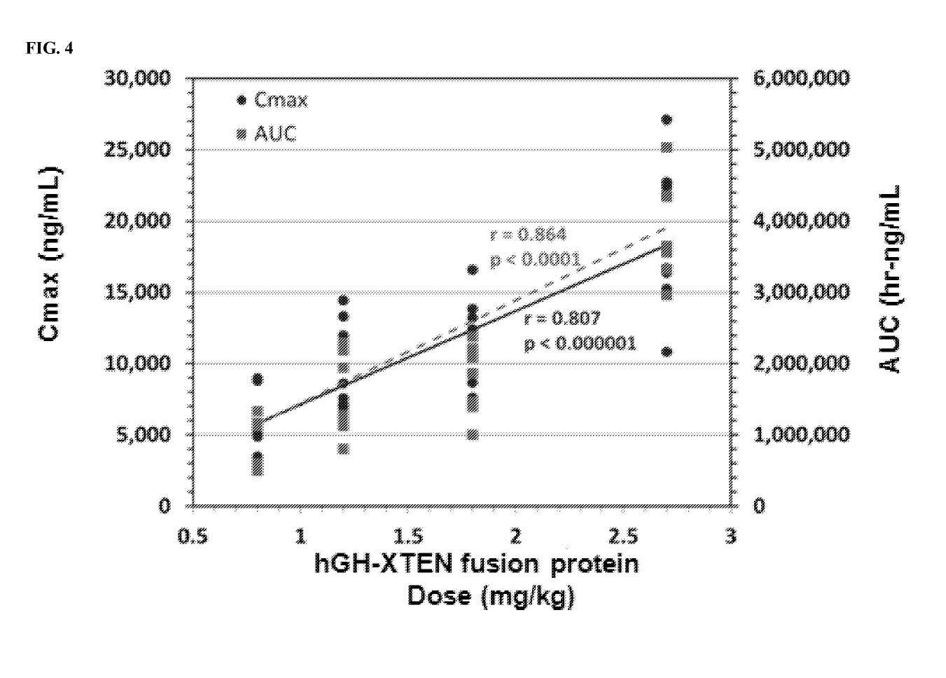

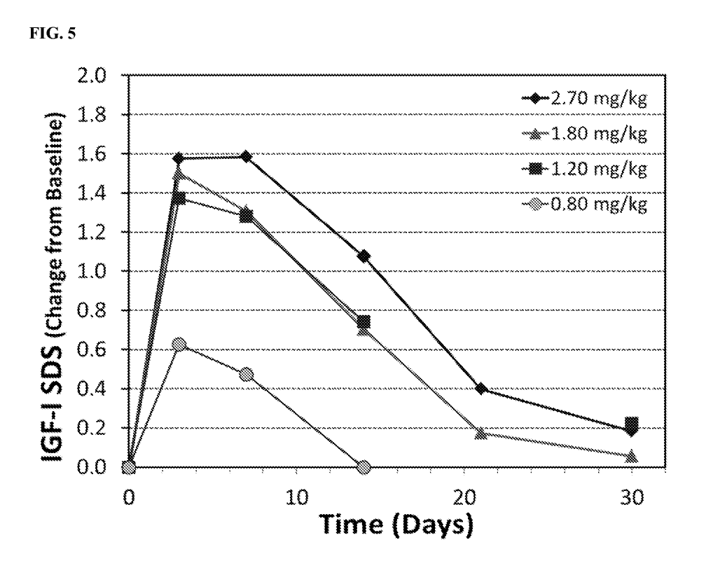

FIG. 4 shows the linear regression for hGH-XTEN fusion protein Cmax (ng/mL) and hGH-XTEN fusion protein AUC (hr-ng/mL) data. These results support that exposure to the fusion protein is linearly proportional to dose.

After a single subcutaneous dose of 2.70 mg/kg of the hGH-XTEN fusion protein, IGF-I SDS was maintained above baseline through Day 30 in 6 of 8 subjects and through Day 22 in the remaining 2 subjects. The prolonged response of IGF-I SDS does not come at the expense of overexposure to IGF-I. An IGF-I SDS>2.0 (2.12) was observed in only one patient at one time point.

FIG. 5 demonstrates a sustained change (from baseline) in IGF-I (mean values) for all doses (0.8 mg/kg (); 1.2 mg/kg (▪); 1.8 mg/kg (▴) and 2.7 mg/kg (♦). IGF-I responses to the hGH-XTEN fusion protein persist for up to 30 days following a single subcutaneous dose.

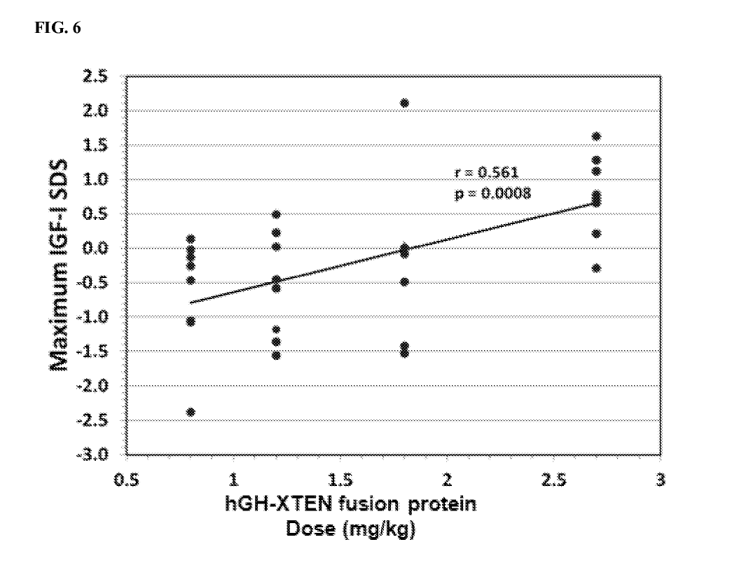

FIG. 6 shows the linear regression for maximum IGF-SDS data for four dose groups and demonstrates that IGF-I responses are linearly related to the dose of the hGH-XTEN fusion protein.

Data from the completed dose levels support the use of a weekly or semimonthly hGH-XTEN fusion protein dose regimen for Phase 2a. Dose escalation continues to support a once monthly hGH-XTEN fusion protein dose regimen for Phase 2a.

### Example 1B

### Single Dose Results

Currently approved growth hormone drugs require daily injections and consequently pose considerable challenges to patients with GHD. In contrast, a human growth hormone analogue (a human growth hormone-XTEN (hGH-XTEN) fusion protein shown as SEQ ID NO:1 (FIG. 1) is being developed to provide up to once-monthly dosing, to facilitate an improvement in patients' ability to adhere to their therapy regimen, and to improve their overall treatment outcomes.

Data were gathered from a Phase 1b/2a Study of a new long-acting human growth hormone (hGH-XTEN fusion protein) in pre-pubertal children with growth hormone deficiency (GHD). The objectives of the Phase 1b study were to evaluate the single dose safety, tolerability of the hGH-XTEN fusion protein in pediatric GHD patients; and to determine PK (hGH-XTEN fusion protein concentrations) and PD (IGF-I, IGFBP-3) profiles over 30 days.

The clinical trial enrolled up to 72 naïve-to-treatment, pre-pubertal children with GHD that was documented by auxologic criteria and two GH stimulation tests. The clinical trial for the hGH-XTEN fusion protein has two stages: a single ascending dose stage (Phase 1b) to determine the safety, PK and PD of the fusion protein doses and to enable selection of dose regimens used in the repeat dose stage (Phase 2a) to obtain 6-month height velocity results. Results from the recently completed Phase 1b dose-escalating stage of the study are available.

The data from the Phase 1b demonstrated that a single dose of the hGH-XTEN fusion protein was very well tolerated in children with GHD and demonstrated that it is possible to safely raise IGF-I to the levels associated with good catch-up growth while using a reduced dosing frequency. The data provided strong support for the continued study of up to once-monthly dosing in the next stage of the trial, which will further determine the 3-month and 6-month height velocities in the GHD patients.

In Phase 1b, the PK and PD (IGF-I) responses over a 30 day period were determined following a single, subcutaneous dose of the hGH-XTEN fusion protein at 6 ascending dose levels. Dosing of the fusion protein began at 0.80 mg/kg, a dose shown to be safe and well tolerated in GHD adults in a previously completed trial, with dose increases to 1.20 mg/kg, 1.80 mg/kg, 2.70 mg/kg, 4.00 mg/kg and 6.00 mg/kg (equivalent to 4.8, 7.4, 11.1, 16.7, 24.7 and 37.0 mcg rhGH per kg per day taken for 30 days). Thus, the doses of hGH-XTEN fusion protein studied in this trial are all below the amount of daily rhGH typically prescribed for these patients. The fusion protein dose selection for Phase 2a was based on safety and IGF-I responses from Phase 1b. Following Phase 2a dose selection, subjects were dosed in each of three dose cohorts for the determination of 3-month and 6-month height velocities.

In the Phase 1b portion of the trial, 48 subjects (27 M, 21 F) with mean (SD) age 7.2 (2.2) yrs were studied in 6 dose cohorts (8 per cohort). At screening, mean (SD) HT-SDS was −2.7 (0.6), weight was 18.0 (4.6) kg and IGF-I SDS was −1.8 (0.7). The hGH-XTEN fusion protein plasma concentrations reached a maximum at a mean time of 3 days post-dose, were proportional to dose and remain detectable for up to 30 days from a single dose in all subjects tested. Maximal changes in IGF-I SDS occurred between 2 to 14 days after a single dose on Day 1. The amplitude and duration of IGF-I responses increased with increasing fusion protein dose. The increase in average IGF-1 SDS over 30 days was also proportional to dose and sufficient to support up to once-monthly dosing of the hGH-XTEN fusion protein. Importantly, the prolonged IGF-I responses did not come at the expense of over-exposure to high IGF-I levels, where only a single value of IGF-I SDS in each of two patients has exceeded +2. All related adverse events that have been reported were mild and transient, with no serious or unexpected adverse events reported.

In sum, single doses of the hGH-XTEN fusion protein from 0.8 to 6.0 mg/kg were safe and well tolerated when administered to 48 pre-pubertal children with GHD. In addition, dose proportional increases in hGH-XTEN fusion protein levels and IGF-I responses were observed, indicating the flexibility for selecting doses and dose regimens of up to once-per-month dosing. Consequently, the hGH-XTEN fusion protein is a long-acting rhGH with the potential for up to monthly-dosing intervals in children with GHD.

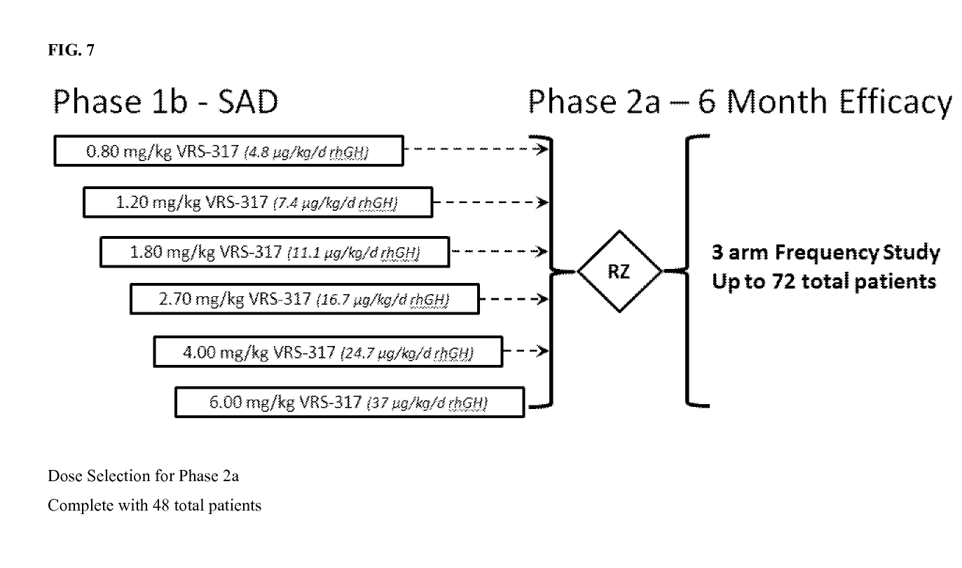

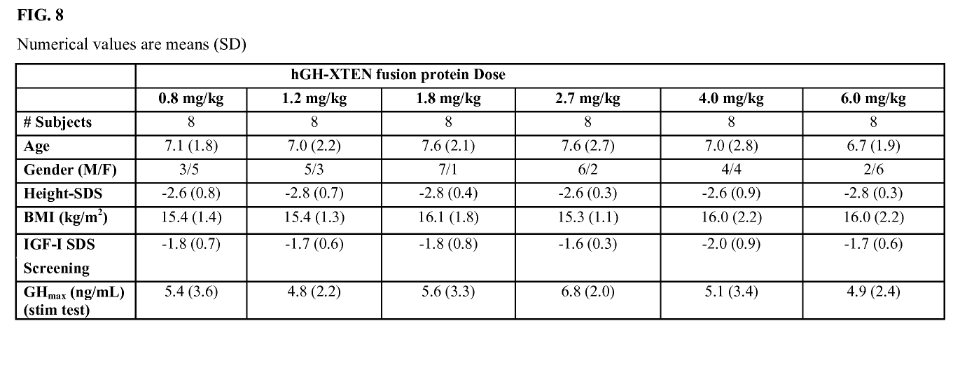

FIG. 7 summarizes the design for the Phase 1b/2a study of a human growth hormone-XTEN (hGH-XTEN) fusion protein in pediatric patients. The hGH-XTEN fusion protein doses equivalent in recombinant hGH (rhGH) mass to 5-37 μg/kd/d taken for 30 days.

FIG. 8 provides a table showing the Clinical Characteristics of Completed Dosing Groups; Numerical values are means (SD).

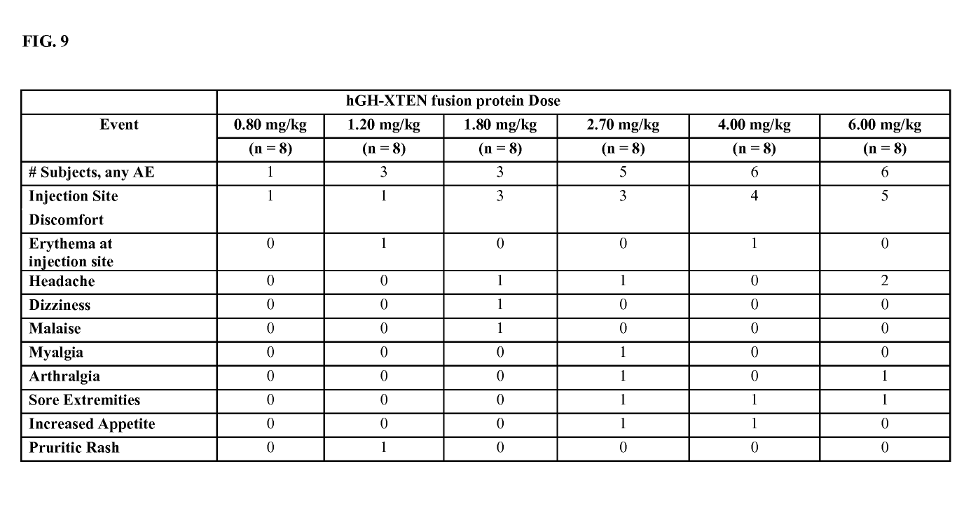

FIG. 9 provides a table showing related adverse events considered as possibly, probably or definitely related to study drug in dose level groups 1-6. All related AE are mild (CTCAE Grade 1) and transient. No SAE, No unexpected AE, No patient withdrawals, No lipoatrophy, No nodules.

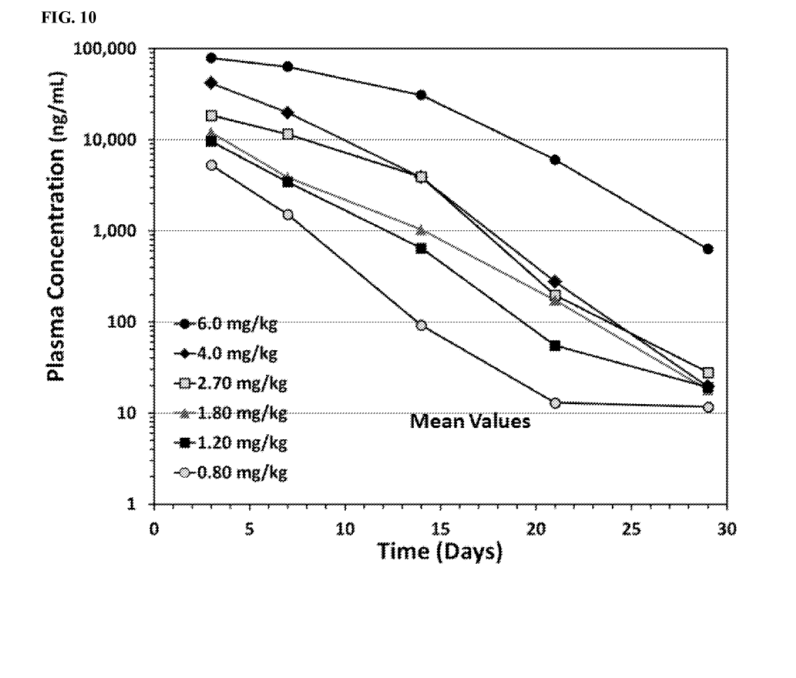

FIG. 10 shows the hGH-XTEN fusion protein plasma concentration (ng/mL) mean values (preliminary PK from Phase 1b).

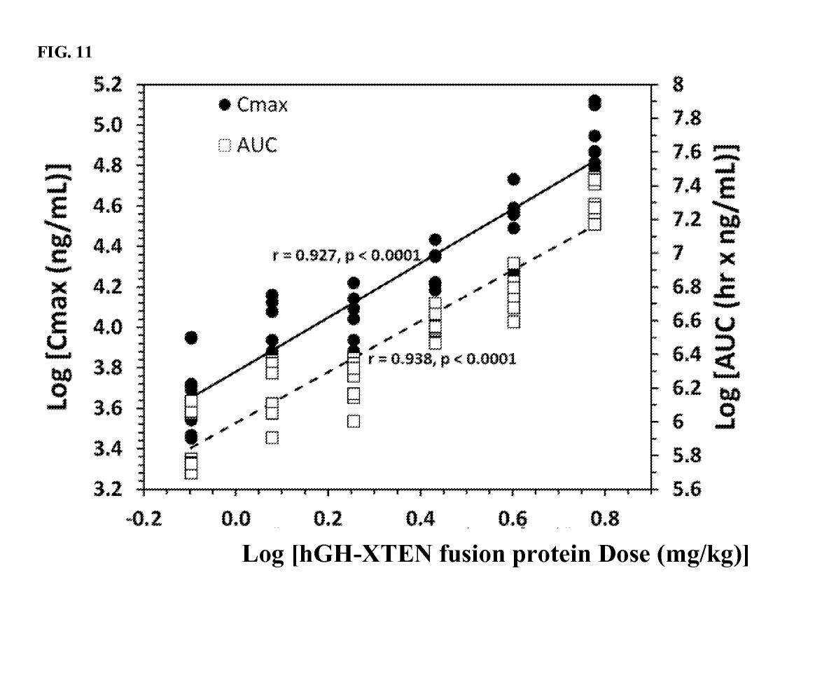

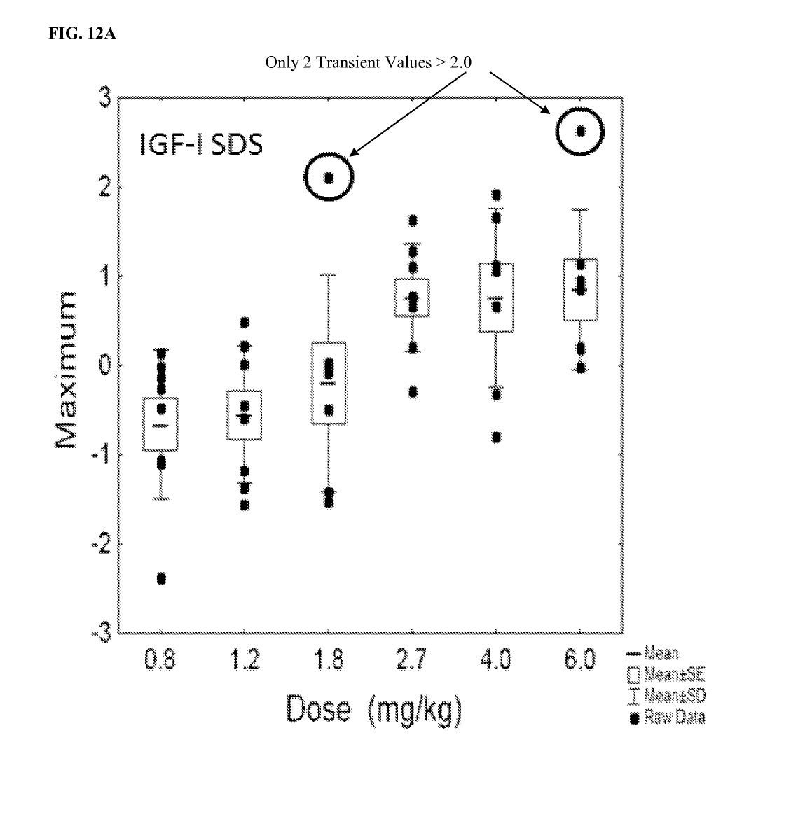

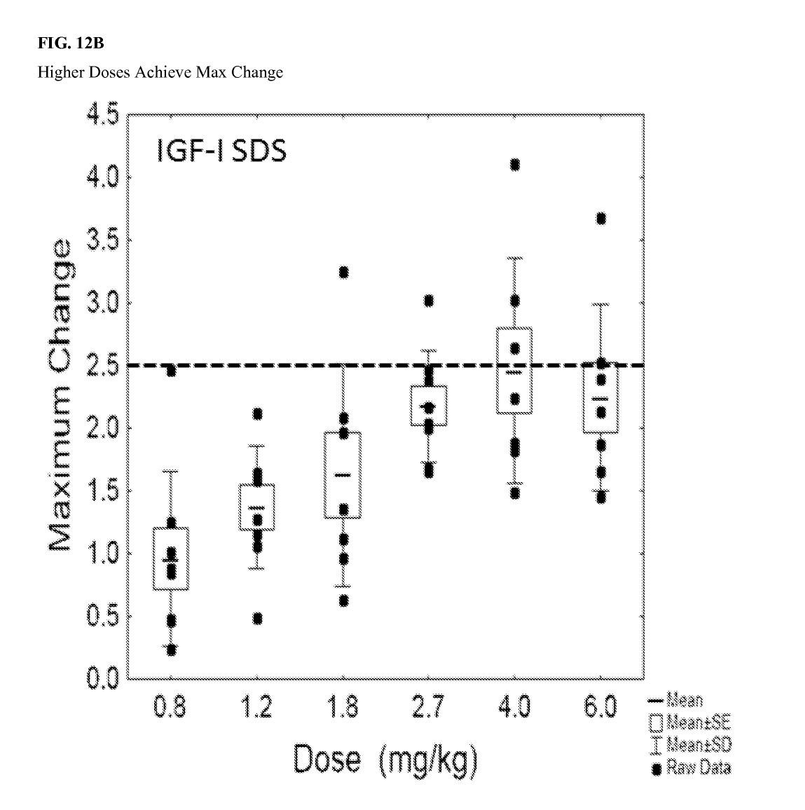

FIG. 11 shows the hGH-XTEN fusion protein Cmax (ng/mL) and hGH-XTEN fusion protein AUC (hr-ng/mL) (dose proportionality).

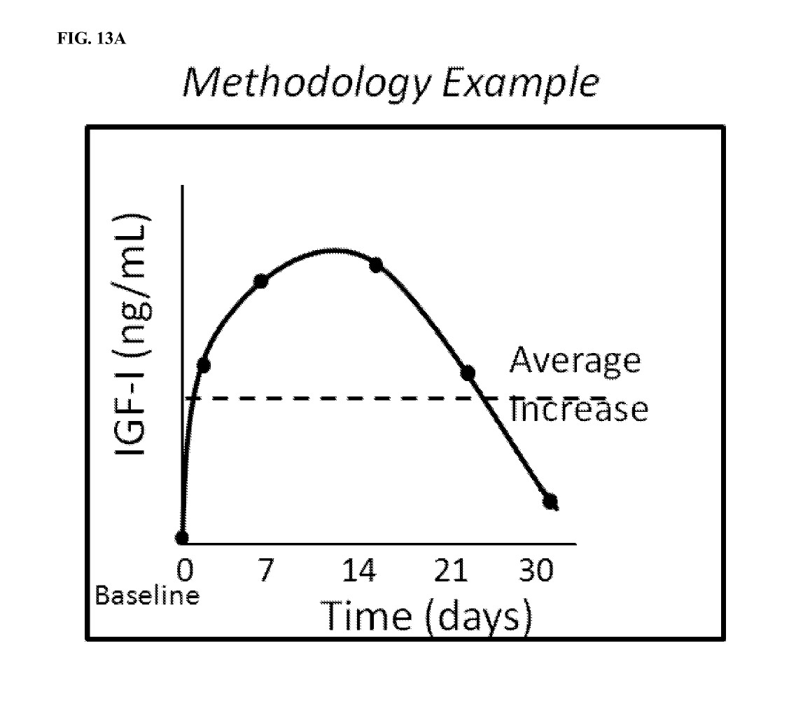

FIG. 12A-B show IGF-I SDS responses to single doses of the fusion protein.

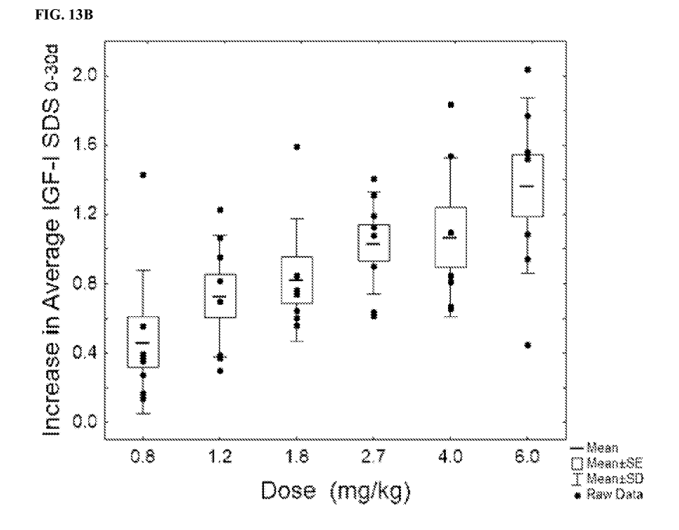

FIG. 13A-B show an increase from Baseline in Monthly Average IGF-I SDS (Single Dose). An increase in average IGF-I SDS increases with increasing dose (p<0.00001). A desired monthly IGF-I profile achieved.

A single dose of the hGH-XTEN fusion protein from 0.80 to 6.0 mg/kg was safe and well tolerated in pre-pubertal children with GHD. Injection site reactions were mild and transient, no nodules and no lipoatrophy. The doses of hGH-XTEN are equivalent to 4.8-37 μg rhGH/kg/d taken for 30 days. The drug exposure parameters (Cmax, AUC) were proportional to dose. The increase in average IGF-I SDS over 30 days was proportional to dose. The increase in monthly average IGF-I was not associated with elevated IGF-I SDS (two transient values>2). The hGH-XTEN fusion protein is a long-acting rhGH with PK/PD attributes for up to monthly dosing.

### Example 2

### Repeat Dosing Results

VRS-317 is a novel fusion protein (M.W. 119 kDa) consisting of rhGH with amino acid sequences (XTEN) attached at the N- and C-termini, SEQ ID NO:1, FIG. 1. In Phase 1 studies in GHD adults and children, VRS-317 concentrations, IGF-I and IGFBP-3 responses were proportional to dose, with drug concentrations and increases in IGF-I and IGFBP-3 still present 30 days after a single subcutaneous injection. Single dose VRS-317 administration has been safe and well tolerated, with minimal injection site discomfort; no new safety signals compared to daily rhGH products have emerged.

A repeat dosing study was conducted to determine the safety, tolerability, height velocity, IGF-I and IGFBP-3 responses after 6 months of VRS-317 treatment. The primary endpoint is mean 6-month height velocity. Subjects were all pre-pubertal and naïve to rhGH treatment. GHD was diagnosed by short stature (HT-SDS<−2), delayed bone age, paired GH stimulation tests (GHmax≦10 ng/mL), a low IGF-I (IGF-I SDS<−1) and absence of other conditions to cause poor growth. Initially, 48 subjects (8/dose cohort) received single doses at one of six VRS-317 dose levels (0.8 to 6.0 mg/kg; equivalent to 4.9 to 37 μg rhGH/kg/d taken for 30 d). Based on observed PK/PD results, 64 subjects were randomized into three dosing arms to evaluate 5.0 mg/kg monthly, 2.5 mg/kg semimonthly or 1.15 mg/kg weekly (cumulative dose of 30 mg/kg/6 m for all). At the start of repeat dosing, the subjects (37 M/27 F) had a mean (SD) age of 7.8 (2.4) yrs, HT-SDS of −2.5 (0.5) and IGF-I SDS of −1.7 (0.8).

With more than 465 injections administered to date, discomfort at injection sites has been mild (Grade 1), transient (generally <30 min) and reported in only 22% of subjects. No nodule formation or lipoatrophy were noted at injection sites. There have been no related serious adverse events (SAEs) or unexpected AE. Other related AE have been mild and transient and of the type expected when rhGH is initiated in children naïve to rhGH treatment (e.g., musculoskeletal pain in 5 subjects, headache in 1 subject). Peak IGF-I SDS levels are greatest with monthly dosing but not >3 and in only 2 cases transiently exceeded 2 (2.01 and 2.12). Mean trough IGF-I SDS levels remain above baseline at Day 30 in all dosing groups. After 2 months of dosing, peak IGF-I levels are generally higher than after the first dose, suggesting that repeat VRS-317 dosing may augment IGF-I responses.

In conclusion, at doses equivalent in rhGH mass to approximately 30 μg rhGH/kg/d, repeat dosing with VRS-317 was found to be safe and well tolerated in pre-pubertal GHD children and maintains mean IGF-I increases over baseline without IGF-I overexposure when given at weekly, semimonthly or monthly intervals. Repeat VRS-317 dosing may augment the IGF-I response seen with initial dosing.

### Example 3

### Three-Month Results

At VRS-317 doses equivalent to daily rhGH of approximately 30 μg rhGH/kg/day, repeat dosing of VRS-317 in Phase 2a to date has been found to be safe and well tolerated in pre-pubertal GHD children and maintains mean IGF-I increases over baseline and within the therapeutic range without IGF-I overexposure when given at weekly, semimonthly and monthly intervals. There have been no related serious adverse events or unexpected adverse events. Other related adverse events have been primarily mild and transient and of the type expected when rhGH is initiated in children naïve to rhGH treatment. With more than 1000 injections administered to date, discomfort at injection sites has occurred in the minority of patients and have been mild and transient. Nodule formation or lipoatrophy has not been observed at injection sites. Peak IGF-I SDS levels have been the greatest with monthly dosing but do not exceed 3 and in only 3 cases transiently exceeded 2. Mean trough IGF-I SDS levels remain above baseline at Day 30 in all dosing groups. After 2 months of dosing, peak IGF-I levels have been generally higher than after the first dose, suggesting that repeat VRS-317 dosing may augment IGF-I responses. The mean annualized 3 month height velocities (a.k.a., growth velocities) from GHD children in the Phase 2a are comparable to the historical age-matched controls administered a comparable dose of daily rhGH (33 μg rhGH/kg/day). Overall, results to date in the Phase 2a clinical trial of GHD children indicate that VRS-317 has a comparable safety and efficacy profile to historical studies of daily rhGH administered at comparable doses.

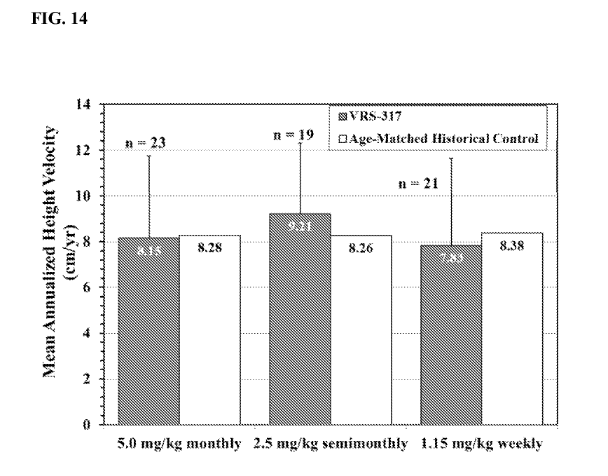

FIG. 14 shows mean annualized height velocities for age-matched historical controls and VRS-317 treated patients.

1. A method of treating human pediatric growth hormone deficiency (PGHD) in a pediatric patient, comprising administering to the pediatric patient with PGHD a human growth hormone-XTEN (hGH-XTEN) fusion protein comprising an amino acid sequence having at least about 90% sequence identity to SEQ ID NO:1, as a therapeutically effective bodyweight adjusted bolus dose between about 0.80 mg/kg and about 6.3 mg/kg.

2. The method of claim 1, wherein the bolus dose is administered once a month, twice a month, three times a month, or four times a month.

3. The method of claim 2, wherein the bolus dose is administered monthly.

4. The method of claim 2, wherein the bolus dose is administered every two weeks or semimonthly.

5. The method of claim 1 or 2, wherein the bolus dose is administered subcutaneously.

6. The method of claim 1, wherein the method is effective to achieve a height velocity equivalent to 7 cm/yr to 12 cm/yr in a pediatric patient.

7. The method of claim 1, wherein the method is effective to achieve a height velocity equivalent to 8 cm/yr to 11 cm/yr in a pediatric patient.

8. The method of claim 6 or 7, wherein the height velocity is achieved after at least 3 months, at least 6 months, or at least 12 months of dosing in the pediatric patient.

9. The method of claim 6 or 7, wherein the height velocity achieved is a first year height velocity.

10. The method of claim 1, wherein the method is not inferior to achieve a height velocity in a pediatric patient compared with that achieved using daily injections of hGH not linked to XTEN over the same period.

11. The method of claim 6 or 7, wherein the bolus dose is selected from about 0.8 mg/kg to about 1.5 mg/kg, about 1.8 mg/kg to about 3.2 mg/kg, or about 3.5 mg/kg to about 6.3 mg/kg.

12. (canceled)

13. The method of claim 1, wherein the pediatric patient maintains an increase from baseline serum IGF-I standard deviation score (SDS) of at least 1.0 for at least about 7 days, at least about 14 days, at least about 21 days, or at least about 30 days following administration.

14. The method of claim 1, wherein the pediatric patient maintains an increase from baseline serum IGF-I standard deviation score (SDS) of at least 1.0 for at least about 14 days, or at least about 30 days following administration.

15. The method of claim 1, wherein the pediatric patient has a serum IGF-I standard deviation score (SDS) between about −2.0 and about 2.0 following administration.

16. The method of claim 15, wherein the IGF-I SDS is selected from the group consisting of greater than about −1.5 to about 2.0, greater than about −1.0 to about 2.0, greater than about −0.5 to about 2.0, greater than about 0 to about 2.0, greater than about 0.5 to about 2.0, greater than about 1.0 to about 2.0, and greater than about 1.5 to about 2.0.

17. The method of claim 15, wherein the IGF-I SDS is selected from the group consisting of greater than about −1.0 to about 2.0, greater than about 0 to about 2.0, and greater than about 1.0 to about 2.0.

18. The method of claim 15, wherein the pediatric patient exhibits said serum IGF-I SDS following administration of the bolus dose, wherein the administration is once a month, twice a month, three times a month, or four times a month.

19. The method of claim 18, wherein the pediatric patient exhibits said serum IGF-I SDS following administration of the bolus dose, wherein the administration is every two weeks, semimonthly, or monthly.

20. The method of claim 19, wherein the pediatric patient exhibits said serum IGF-I SDS following administration of at least a second, or a third, or a fourth bolus dose.

21. The method of claim 15, wherein the bolus dose is effective to maintain the pediatric patient's serum IGF-I SDS between about −2.0 and about 2.0 for at least about 7 days, at least about 14 days, at least about 21 days, at least about 30 days, or at least about one month following administration.

22. The method of claim 15, wherein the bolus dose is effective to maintain the pediatric patient's serum IGF-I SDS between about −2.0 and about 2.0 for at least about 14 days, at least about 21 days, or at least about 30 days following administration.

23. The method of claim 1, comprising administering the hGH-XTEN fusion protein in an initial bolus dose between about 0.8 mg/kg and about 6.3 mg/kg and a plurality of subsequent bolus doses of the hGH-XTEN fusion protein between about 0.8 mg/kg and about 6.3 mg/kg.

24. The method of any one of claim 22 or 23, wherein the IGF-I SDS is maintained between about −2.0 and about 2.0 following administration of a first, or a second, or a third, or a fourth bolus dose.

25. The method of claim 1, wherein the bolus dose is selected from the group consisting of about 0.8 mg/kg, about 1.0 mg/kg, about 1.2 mg/kg, about 1.4 mg/kg, about 1.6 mg/kg, about 1.8 mg/kg, about 2.0 mg/kg, about 2.2 mg/kg, about 2.4 mg/kg, about 2.6 mg/kg, about 2.7 mg/kg, about 2.8 mg/kg, about 3 mg/kg, about 3.2 mg/kg, about 3.4 mg/kg, about 3.6 mg/kg, about 3.8 mg/kg, about 4.0 mg/kg, about 4.2 mg/kg, about 4.4 mg/kg, about 4.6 mg/kg, about 4.8 mg/kg, about 5.0 mg/kg, about 5.2 mg/kg, about 5.4 mg/kg, about 5.6 mg/kg, about 5.8 mg/kg, about 6.0 mg/kg, and about 6.3 mg/kg.

26. The method of claim 1, wherein the bolus dose is about 0.8 mg/kg to about 1.5 mg/kg.

27. The method of claim 1, wherein the bolus dose is about 1.8 mg/kg to about 3.2 mg/kg.

28. The method of claim 1, wherein the bolus dose is about 3.5 mg/kg to about 6.3 mg/kg.

29. The method of claim 1, wherein the hGH-XTEN fusion protein comprises the amino acid sequence of SEQ ID NO:1.

30. The method of claim 1, wherein the hGH-XTEN fusion protein has at least about 91%, or at least about 92%, or at least about 93%, or at least about 94%, or at least about 95%, or at least about 96%, or at least about 97%, or at least about 98%, or at least about 99% sequence identity to SEQ ID NO:1.

31. A method of treating human pediatric growth hormone deficiency (PGHD) in a human pediatric patient, comprising administering to the patient with PGHD a human growth hormone-XTEN (hGH-XTEN) fusion protein comprising an amino acid sequence having at least about 90% sequence identity to SEQ ID NO:1 as a therapeutically effective bodyweight adjusted bolus dose that is effective to maintain the patient's serum IGF-I standard deviation score (SDS) between about −2.0 and about 2.0 for at least 7 days after administration of the bolus dose.

32. The method of claim 31, wherein the bolus dose is between about 0.8 mg/kg and about 6.3 mg/kg.

33. (canceled)

34. The method of claim 31, wherein the bolus dose is effective to maintain the pediatric patient's serum IGF-I SDS between about −2.0 and about 2.0 for at least about 14 days, at least about 21 days, or at least about 30 days following administration.

35. The method of claim 31, wherein the bolus dose is effective to maintain the pediatric patient's serum IGF-I SDS between about −0.5 and about 2.0 for at least about 14 days, or at least about 30 days following administration.

36. A pediatric bolus dose of an hGH-XTEN fusion protein comprising an amino acid sequence having at least about 90% sequence identity to SEQ ID NO:1, wherein the bolus dose is a therapeutically effective bodyweight adjusted bolus dose comprising between about 0.8 mg/kg and about 6.3 mg/kg of hGH-XTEN fusion protein.

37. The bolus dose of claim 36 for use in treating human pediatric growth hormone deficiency (PGHD) in a pediatric patient in need.

38. The bolus dose of claim 36, wherein the hGH-XTEN fusion protein comprises the amino acid sequence of SEQ ID NO:1.

39. The bolus dose of claim 36, which is formulated for subcutaneous administration.

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. A kit for the treatment of pediatric growth hormone deficiency (PGHD) comprising
(i) a container which holds a pharmaceutical composition comprising a human growth hormone-XTEN (hGH-XTEN) fusion protein comprising an amino acid sequence having at least about 90% sequence identity to SEQ ID NO:1, and
(ii) a package insert associated with said container, wherein the package insert indicates that said composition is for the treatment of pediatric growth hormone deficiency (PGHD) in a pediatric patient by administration of an initial dose of the hGH-XTEN fusion protein between about 0.8 mg/kg and about 6.3 mg/kg and a plurality of subsequent doses of the hGH-XTEN fusion protein between about 0.8 mg/kg and about 6.3 mg/kg, wherein the doses are administered once a month, twice a month, three times a month, or four times a month.

52. The kit of claim 51, wherein the container further comprises a pharmaceutically acceptable carrier.

53. (canceled)

54. (canceled)

55. The method of claim 31, comprising administering the hGH-XTEN fusion protein in an initial bolus dose between about 0.8 mg/kg and about 6.3 mg/kg and a plurality of subsequent bolus doses of the hGH-XTEN fusion protein between about 0.8 mg/kg and about 6.3 mg/kg.

56. (canceled)

57. (canceled)

58. The method of claim 10, wherein the hGH-XTEN fusion protein administered is comparable, on a molar basis, to an equivalent amount of an hGH not linked to XTEN and administered to a pediatric patient.

59. The method of claim 1, wherein the method is effective to maintain the pediatric patient's height velocity within at least about 10%, at least about 20%, or at least about 30% of that compared to the height velocity achieved in pediatric patients administered daily injections of hGH not linked to XTEN of an equivalent amount, on a molar basis, over a comparable dose period.

60. The method of claim 1 or 31, wherein the bolus dose of the hGH-XTEN administered provides an area under the curve of about 5.6 Log [AUC (h ng/mL)] to about 7.6 Log [AUC (h ng/mL)].

61. The method of claim 1 or 31, wherein the bolus dose of the hGH-XTEN administered provides hGH-XTEN plasma concentration of at least 10 ng/ml.

62. The method of claim 61, wherein the bolus dose of the hGH-XTEN maintains said plasma concentration for at least about 7 days, at least about 14 days or at least about 30 days following administration.


SEQUENCE LISTING (41 sequences)

SEQ ID NO: 1 - protein, 1250 aa, Artificial Sequence
  Description of Artificial Sequence Synthetic polypeptide
AEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGTSESATPESGPGTSTEPSEGSAPGTSTEPSEGSAPGTSESATPESGPGTSTEPSEGSAPGTSTEPSEGSAPGTSESATPESGPGTSTEPSEGSAPGTSTEPSEGSAPGTSTEPSEGSAPGSPAGSPTSTEEGTSTEPSEGSAPG

SEQ ID NO: 2 - protein, 1071 aa, Artificial Sequence
  Description of Artificial Sequence Synthetic polypeptide
GGSPGTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGSPAGSPTSTEEGSSTPSGATGSPGSSPSASTGTGPGASPGTSSTGSPGTSESATPESGPGTSTEPSEGSAPGTSTEPSEGSAPGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 3 - protein, 768 aa, Artificial Sequence
  Description of Artificial Sequence Synthetic polypeptide
GEGSGEGSEGEGSEGSGEGEGSEGSGEGEGGSEGSEGEGSEGSGEGEGGEGSGEGEGSGEGSEGEGGGEGSEGEGSGEGGEGEGSEGGSEGEGGSEGGEGEGSEGSGEGEGSEGGSEGEGSEGGSEGEGSEGSGEGEGSEGSGEGEGSEGSGEGEGSEGSGEGEGSEGGSEGEGGSEGSEGEGSGEGSEGEGGSEGSEGEGGGEGSEGEGSGEGSEGEGGSEGSEGEGGSEGSEGEGGEGSGEGEGSEGSGEGEGSGEGSEGEGSEGSGEGEGSEGSGEGEGGSEGSEGEGSGEGSEGEGSEGSGEGEGSEGSGEGEGGSEGSEGEGGSEGSEGEGGSEGSEGEGGEGSGEGEGSEGSGEGEGSGEGSEGEGSEGSGEGEGSEGSGEGEGGSEGSEGEGSEGSGEGEGGEGSGEGEGSGEGSEGEGGGEGSEGEGSEGSGEGEGSEGSGEGEGSEGGSEGEGGSEGSEGEGSEGGSEGEGSEGGSEGEGSEGSGEGEGSEGSGEGEGSGEGSEGEGGSEGGEGEGSEGGSEGEGSEGGSEGEGGEGSGEGEGGGEGSEGEGSEGSGEGEGSGEGSEGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 4 - protein, 1104 aa, Artificial Sequence
  Description of Artificial Sequence Synthetic polypeptide
AEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 5 - protein, 1394 aa, Artificial Sequence
  Description of Artificial Sequence Synthetic polypeptide
AEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPG

SEQ ID NO: 6 - protein, 1067 aa, Artificial Sequence
  Description of Artificial Sequence Synthetic polypeptide
GTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGSPAGSPTSTEEGSSTPSGATGSPGSSPSASTGTGPGASPGTSSTGSPGTSESATPESGPGTSTEPSEGSAPGTSTEPSEGSAPGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 7 - DNA, 3753 bp, Artificial Sequence
  Description of Artificial Sequence Synthetic polynucleotide
atggctgaacctgctggctctccaacctccactgaggaaggtaccccgggtagcggtactgcttcttcctctccaggtagctctaccccttctggtgcaaccggctctccaggtgcttctccgggcaccagctctaccggttctccaggtagcccggctggctctcctacctctactgaggaaggtacttctgaaagcgctactcctgagtctggtccaggtacctctactgaaccgtccgaaggtagcgctccaggtagcccagcaggctctccgacttccactgaggaaggtacttctactgaaccttccgaaggcagcgcaccaggtacctctactgaaccttctgagggcagcgctccaggtacttctgaaagcgctaccccggaatctggcccaggtagcgaaccggctacttctggttctgaaaccccaggtagcgaaccggctacctccggttctgaaactccaggtagcccggcaggctctccgacctctactgaggaaggtacttctgaaagcgcaaccccggagtccggcccaggtacctctaccgaaccgtctgagggcagcgcaccaggtacttctaccgaaccgtccgagggtagcgcaccaggtagcccagcaggttctcctacctccaccgaggaaggtacttctaccgaaccgtccgagggtagcgcaccaggtacctctactgaaccttctgagggcagcgctccaggtacttctgaaagcgctaccccggagtccggtccaggtacttctactgaaccgtccgaaggtagcgcaccaggtacttctgaaagcgcaacccctgaatccggtccaggtagcgaaccggctacttctggctctgagactccaggtacttctaccgaaccgtccgaaggtagcgcaccaggtacttctactgaaccgtctgaaggtagcgcaccaggtacttctgaaagcgcaaccccggaatccggcccaggtacctctgaaagcgcaaccccggagtccggcccaggtagccctgctggctctccaacctccaccgaagaaggtacctctgaaagcgcaacccctgaatccggcccaggtagcgaaccggcaacctccggttctgaaaccccaggtacctctgaaagcgctactccggagtctggcccaggtacctctactgaaccgtctgagggtagcgctccaggtacttctactgaaccgtccgaaggtagcgcaccaggtacttctaccgaaccgtccgaaggcagcgctccaggtacctctactgaaccttccgagggcagcgctccaggtacctctaccgaaccttctgaaggtagcgcaccaggtacttctaccgaaccgtccgagggtagcgcaccaggtagcccagcaggttctcctacctccaccgaggaaggtacttctaccgaaccgtccgagggtagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtacttctgaaagcgctactcctgagtccggcccaggtagcccggctggctctccgacttccaccgaggaaggtagcccggctggctctccaacttctactgaagaaggtagcccggcaggctctccgacctctactgaggaaggtacttctgaaagcgcaaccccggagtccggcccaggtacctctaccgaaccgtctgagggcagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtagccctgctggctctccaacctccaccgaagaaggtacctctgaaagcgcaacccctgaatccggcccaggtagcgaaccggcaacctccggttctgaaaccccaggtacttctgaaagcgctactcctgagtccggcccaggtagcccggctggctctccgacttccaccgaggaaggtagcccggctggctctccaacttctactgaagaaggtacttctaccgaaccttccgagggcagcgcaccaggtacttctgaaagcgctacccctgagtccggcccaggtacttctgaaagcgctactcctgaatccggtccaggtacttctgaaagcgctaccccggaatctggcccaggtagcgaaccggctacttctggttctgaaaccccaggtagcgaaccggctacctccggttctgaaactccaggtagcccagcaggctctccgacttccactgaggaaggtacttctactgaaccttccgaaggcagcgcaccaggtacctctactgaaccttctgagggcagcgctccaggtagcgaacctgcaacctctggctctgaaaccccaggtacctctgaaagcgctactcctgaatctggcccaggtacttctactgaaccgtccgagggcagcgcaccaggttttccgactattccgctgtctcgtctgtttgataatgctatgctgcgtgcgcaccgtctgcaccagctggcctttgatacttaccaggaatttgaagaagcctacattcctaaagagcagaagtactctttcctgcaaaacccacagacttctctctgcttcagcgaatctattccgacgccttccaatcgcgaggaaactcagcaaaagtccaatctggaactactccgcatttctctgcttctgattcagagctggctagaaccagtgcaatttctgcgttccgtcttcgccaatagcctagtttatggcgcatccgacagcaacgtatacgatctcctgaaagatctcgaggaaggcattcagaccctgatgggtcgtctcgaggatggctctccgcgtactggtcagatcttcaagcagacttactctaaatttgatactaacagccacaatgacgatgcgcttctaaaaaactatggtctgctgtattgttttcgtaaagatatggacaaagttgaaaccttcctgcgtattgttcagtgtcgttccgttgagggcagctgtggtttctaaggtggtagcgaaccggcaacttccggctctgaaaccccaggtacttctgaaagcgctactcctgagtctggcccaggtagcgaacctgctacctctggctctgaaaccccaggtagcccggcaggctctccgacttccaccgaggaaggtacctctactgaaccttctgagggtagcgctccaggtagcgaaccggcaacctctggctctgaaaccccaggtagcgaacctgctacctccggctctgaaactccaggtagcgaaccggctacttccggttctgaaactccaggtacctctaccgaaccttccgaaggcagcgcaccaggtacttctgaaagcgcaacccctgaatccggtccaggtagcgaaccggctacttctggctctgagactccaggtacttctaccgaaccgtccgaaggtagcgcacca

SEQ ID NO: 8 - DNA, 3213 bp, Artificial Sequence
  Description of Artificial Sequence Synthetic polynucleotide
ggtgggtctccaggtacttctactgaaccgtctgaaggcagcgcaccaggtagcgaaccggctacttccggttctgaaaccccaggtagcccagcaggttctccaacttctactgaagaaggttctaccagctctaccgcagaatctcctggtccaggtacctctactccggaaagcggctctgcatctccaggttctactagcgaatctccttctggcactgcaccaggttctactagcgaatccccgtctggtactgctccaggtacttctactcctgaaagcggttccgcttctccaggtacctctactccggaaagcggttctgcatctccaggtagcgaaccggcaacctccggctctgaaaccccaggtacctctgaaagcgctactcctgaatccggcccaggtagcccggcaggttctccgacttccactgaggaaggtacctctactgaaccttctgagggcagcgctccaggtacttctgaaagcgctaccccggagtccggtccaggtacttctactgaaccgtccgaaggtagcgcaccaggtacttctaccgaaccgtccgagggtagcgcaccaggtagcccagcaggttctcctacctccaccgaggaaggtacttctaccgaaccgtccgagggtagcgcaccaggtacttctaccgaaccttccgagggcagcgcaccaggtacttctgaaagcgctacccctgagtccggcccaggtacttctgaaagcgctactcctgaatccggtccaggtacctctactgaaccttccgaaggcagcgctccaggtacctctaccgaaccgtccgagggcagcgcaccaggtacttctgaaagcgcaacccctgaatccggtccaggtacttctactgaaccttccgaaggtagcgctccaggtagcgaacctgctacttctggttctgaaaccccaggtagcccggctggctctccgacctccaccgaggaaggtagctctaccccgtctggtgctactggttctccaggtactccgggcagcggtactgcttcttcctctccaggtagctctaccccttctggtgctactggctctccaggtacctctaccgaaccgtccgagggtagcgcaccaggtacctctactgaaccgtctgagggtagcgctccaggtagcgaaccggcaacctccggttctgaaactccaggtagccctgctggctctccgacttctactgaggaaggtagcccggctggttctccgacttctactgaggaaggtacttctaccgaaccttccgaaggtagcgctccaggtgcaagcgcaagcggcgcgccaagcacgggaggtacttctgaaagcgctactcctgagtccggcccaggtagcccggctggctctccgacttccaccgaggaaggtagcccggctggctctccaacttctactgaagaaggttctaccagctctaccgctgaatctcctggcccaggttctactagcgaatctccgtctggcaccgcaccaggtacttcccctagcggtgaatcttctactgcaccaggtacccctggcagcggtaccgcttcttcctctccaggtagctctaccccgtctggtgctactggctctccaggttctagcccgtctgcatctaccggtaccggcccaggtagcgaaccggcaacctccggctctgaaactccaggtacttctgaaagcgctactccggaatccggcccaggtagcgaaccggctacttccggctctgaaaccccaggttccaccagctctactgcagaatctccgggcccaggttctactagctctactgcagaatctccgggtccaggtacttctcctagcggcgaatcttctaccgctccaggtagcgaaccggcaacctctggctctgaaactccaggtagcgaacctgcaacctccggctctgaaaccccaggtacttctactgaaccttctgagggcagcgcaccaggttctaccagctctaccgcagaatctcctggtccaggtacctctactccggaaagcggctctgcatctccaggttctactagcgaatctccttctggcactgcaccaggtacttctaccgaaccgtccgaaggcagcgctccaggtacctctactgaaccttccgagggcagcgctccaggtacctctaccgaaccttctgaaggtagcgcaccaggtagctctactccgtctggtgcaaccggctccccaggttctagcccgtctgcttccactggtactggcccaggtgcttccccgggcaccagctctactggttctccaggtagcgaacctgctacctccggttctgaaaccccaggtacctctgaaagcgcaactccggagtctggtccaggtagccctgcaggttctcctacctccactgaggaaggtagctctactccgtctggtgcaaccggctccccaggttctagcccgtctgcttccactggtactggcccaggtgcttccccgggcaccagctctactggttctccaggtacctctgaaagcgctactccggagtctggcccaggtacctctactgaaccgtctgagggtagcgctccaggtacttctactgaaccgtccgaaggtagcgcaccaggttttccgactattccgctgtctcgtctgtttgataatgctatgctgcgtgcgcaccgtctgcaccagctggcctttgatacttaccaggaatttgaagaagcctacattcctaaagagcagaagtactctttcctgcaaaacccacagacttctctctgcttcagcgaatctattccgacgccttccaatcgcgaggaaactcagcaaaagtccaatctggaactactccgcatttctctgcttctgattcagagctggctagaaccagtgcaatttctgcgttccgtcttcgccaatagcctagtttatggcgcatccgacagcaacgtatacgatctcctgaaagatctcgaggaaggcattcagaccctgatgggtcgtctcgaggatggctctccgcgtactggtcagatcttcaagcagacttactctaaatttgatactaacagccacaatgacgatgcgcttctaaaaaactatggtctgctgtattgttttcgtaaagatatggacaaagttgaaaccttcctgcgtattgttcagtgtcgttccgttgagggcagctgtggtttc

SEQ ID NO: 9 - DNA, 2304 bp, Artificial Sequence
  Description of Artificial Sequence Synthetic polynucleotide
ggtgagggttctggcgaaggttccgaaggtgagggctccgaaggatctggcgaaggtgagggttccgaaggttctggcgaaggtgaaggcggttctgagggatccgaaggtgaaggctccgaaggatctggcgaaggtgaaggtggtgaaggttctggcgaaggtgagggatctggcgaaggctctgaaggtgaaggtggtggtgaaggctctgaaggtgaaggatctggtgaaggtggcgaaggtgagggatctgaaggcggctccgaaggtgaaggcggatctgaaggcggcgaaggtgaaggttccgaaggttctggtgaaggtgaaggatctgaaggtggctccgaaggtgaaggatctgaaggcggttccgaaggtgagggctctgaaggttctggcgaaggtgaaggctctgaaggatctggtgaaggtgaaggttccgaaggttctggtgaaggtgaaggttccgaaggttctggcgaaggtgaaggttctgaaggtggctctgaaggtgaaggcggctctgaaggatccgaaggtgaaggttctggtgaaggctctgaaggtgaaggcggctctgagggttccgaaggtgaaggcggaggcgaaggttctgaaggtgagggatctggtgaaggttctgaaggtgaaggcggttctgaaggttccgaaggtgaaggtggctctgagggatccgaaggtgaaggtggcgaaggatctggtgaaggtgaaggttctgaaggttctggcgaaggtgagggttctggcgaaggttccgaaggtgagggctccgaaggatctggcgaaggtgagggttccgaaggttctggcgaaggtgaaggcggttctgagggatccgaaggtgagggttctggcgaaggttccgaaggtgagggctccgaaggatctggcgaaggtgagggttccgaaggttctggcgaaggtgaaggcggttctgagggatccgaaggtgaaggcggttctgaaggttccgaaggtgaaggtggctctgagggatccgaaggtgaaggtggcgaaggatctggtgaaggtgaaggttctgaaggttctggcgaaggtgagggttctggcgaaggttccgaaggtgagggctccgaaggatctggcgaaggtgagggttccgaaggttctggcgaaggtgaaggcggttctgagggatccgaaggtgaaggctccgaaggatctggcgaaggtgaaggtggtgaaggttctggcgaaggtgagggatctggcgaaggctctgaaggtgaaggtggtggtgaaggctctgaaggtgaaggttccgaaggttctggtgaaggtgaaggttccgaaggttctggcgaaggtgaaggttctgaaggtggctctgaaggtgaaggcggctctgaaggatccgaaggtgaaggatctgaaggtggctccgaaggtgaaggatctgaaggcggttccgaaggtgagggctctgaaggttctggcgaaggtgaaggctctgaaggatctggtgaaggtgaaggatctggcgaaggctccgaaggtgaaggcggttctgaaggtggcgaaggtgaaggatctgaaggtggttccgaaggtgagggatctgaaggtggctctgaaggtgaaggtggcgaaggttctggcgaaggtgaaggtggaggcgaaggttctgaaggtgaaggttccgaaggttctggtgaaggtgagggatctggcgaaggttctgaaggttttccgactattccgctgtctcgtctgtttgataacgctatgctgcgtgcgcaccgtctgcaccagctggcgttcgacacttaccaggaatttgaagaagcgtacattccgaaggaacagaagtactctttcctgcaaaacccgcagacctccctgtgcttcagcgaatctattccgactccgtccaatcgtgaagaaactcagcaaaagtccaatctggagctgctgcgcatctctctgctgctgattcagagctggctggagcctgttcagtttctgcgttccgtcttcgccaacagcctggtttatggtgcttccgacagcaacgtatacgatctgctgaaagatctggaagaaggcattcagaccctgatgggtcgtctggaagatggttctccgcgtactggtcagatcttcaaacaaacttactccaaatttgatactaacagccataacgacgatgctctgctgaaaaactatggtctgctgtattgcttccgcaaggatatggacaaagttgaaaccttcctgcgtattgtgcagtgtcgttccgttgagggcagctgtggtttc

SEQ ID NO: 10 - DNA, 3318 bp, Artificial Sequence
  Description of Artificial Sequence Synthetic polynucleotide
atggctgaacctgctggctctccaacctccactgaggaaggtaccccgggtagcggtactgcttcttcctctccaggtagctctaccccttctggtgcaaccggctctccaggtgcttctccgggcaccagctctaccggttctccaggtagcccggctggctctcctacctctactgaggaaggtacttctgaaagcgctactcctgagtctggtccaggtacctctactgaaccgtccgaaggtagcgctccaggtagcccagcaggctctccgacttccactgaggaaggtacttctactgaaccttccgaaggcagcgcaccaggtacctctactgaaccttctgagggcagcgctccaggtacttctgaaagcgctaccccggaatctggcccaggtagcgaaccggctacttctggttctgaaaccccaggtagcgaaccggctacctccggttctgaaactccaggtagcccggcaggctctccgacctctactgaggaaggtacttctgaaagcgcaaccccggagtccggcccaggtacctctaccgaaccgtctgagggcagcgcaccaggtacttctaccgaaccgtccgagggtagcgcaccaggtagcccagcaggttctcctacctccaccgaggaaggtacttctaccgaaccgtccgagggtagcgcaccaggtacctctactgaaccttctgagggcagcgctccaggtacttctgaaagcgctaccccggagtccggtccaggtacttctactgaaccgtccgaaggtagcgcaccaggtacttctgaaagcgcaacccctgaatccggtccaggtagcgaaccggctacttctggctctgagactccaggtacttctaccgaaccgtccgaaggtagcgcaccaggtacttctactgaaccgtctgaaggtagcgcaccaggtacttctgaaagcgcaaccccggaatccggcccaggtacctctgaaagcgcaaccccggagtccggcccaggtagccctgctggctctccaacctccaccgaagaaggtacctctgaaagcgcaacccctgaatccggcccaggtagcgaaccggcaacctccggttctgaaaccccaggtacctctgaaagcgctactccggagtctggcccaggtacctctactgaaccgtctgagggtagcgctccaggtacttctactgaaccgtccgaaggtagcgcaccaggtacttctaccgaaccgtccgaaggcagcgctccaggtacctctactgaaccttccgagggcagcgctccaggtacctctaccgaaccttctgaaggtagcgcaccaggtacttctaccgaaccgtccgagggtagcgcaccaggtagcccagcaggttctcctacctccaccgaggaaggtacttctaccgaaccgtccgagggtagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtacttctgaaagcgctactcctgagtccggcccaggtagcccggctggctctccgacttccaccgaggaaggtagcccggctggctctccaacttctactgaagaaggtagcccggcaggctctccgacctctactgaggaaggtacttctgaaagcgcaaccccggagtccggcccaggtacctctaccgaaccgtctgagggcagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtagccctgctggctctccaacctccaccgaagaaggtacctctgaaagcgcaacccctgaatccggcccaggtagcgaaccggcaacctccggttctgaaaccccaggtacttctgaaagcgctactcctgagtccggcccaggtagcccggctggctctccgacttccaccgaggaaggtagcccggctggctctccaacttctactgaagaaggtacttctaccgaaccttccgagggcagcgcaccaggtacttctgaaagcgctacccctgagtccggcccaggtacttctgaaagcgctactcctgaatccggtccaggtacttctgaaagcgctaccccggaatctggcccaggtagcgaaccggctacttctggttctgaaaccccaggtagcgaaccggctacctccggttctgaaactccaggtagcccagcaggctctccgacttccactgaggaaggtacttctactgaaccttccgaaggcagcgcaccaggtacctctactgaaccttctgagggcagcgctccaggtagcgaacctgcaacctctggctctgaaaccccaggtacctctgaaagcgctactcctgaatctggcccaggtacttctactgaaccgtccgagggcagcgcaccaggttttccgactattccgctgtctcgtctgtttgataatgctatgctgcgtgcgcaccgtctgcaccagctggcctttgatacttaccaggaatttgaagaagcctacattcctaaagagcagaagtactctttcctgcaaaacccacagacttctctctgcttcagcgaatctattccgacgccttccaatcgcgaggaaactcagcaaaagtccaatctggaactactccgcatttctctgcttctgattcagagctggctagaaccagtgcaatttctgcgttccgtcttcgccaatagcctagtttatggcgcatccgacagcaacgtatacgatctcctgaaagatctcgaggaaggcattcagaccctgatgggtcgtctcgaggatggctctccgcgtactggtcagatcttcaagcagacttactctaaatttgatactaacagccacaatgacgatgcgcttctaaaaaactatggtctgctgtattgttttcgtaaagatatggacaaagttgaaaccttcctgcgtattgttcagtgtcgttccgttgagggcagctgtggtttctaa

SEQ ID NO: 11 - DNA, 4185 bp, Artificial Sequence
  Description of Artificial Sequence Synthetic polynucleotide
atggctgaacctgctggctctccaacctccactgaggaaggtaccccgggtagcggtactgcttcttcctctccaggtagctctaccccttctggtgcaaccggctctccaggtgcttctccgggcaccagctctaccggttctccaggtagcccggctggctctcctacctctactgaggaaggtacttctgaaagcgctactcctgagtctggtccaggtacctctactgaaccgtccgaaggtagcgctccaggtagcccagcaggctctccgacttccactgaggaaggtacttctactgaaccttccgaaggcagcgcaccaggtacctctactgaaccttctgagggcagcgctccaggtacttctgaaagcgctaccccggaatctggcccaggtagcgaaccggctacttctggttctgaaaccccaggtagcgaaccggctacctccggttctgaaactccaggtagcccggcaggctctccgacctctactgaggaaggtacttctgaaagcgcaaccccggagtccggcccaggtacctctaccgaaccgtctgagggcagcgcaccaggtacttctaccgaaccgtccgagggtagcgcaccaggtagcccagcaggttctcctacctccaccgaggaaggtacttctaccgaaccgtccgagggtagcgcaccaggtacctctactgaaccttctgagggcagcgctccaggtacttctgaaagcgctaccccggagtccggtccaggtacttctactgaaccgtccgaaggtagcgcaccaggtacttctgaaagcgcaacccctgaatccggtccaggtagcgaaccggctacttctggctctgagactccaggtacttctaccgaaccgtccgaaggtagcgcaccaggtacttctactgaaccgtctgaaggtagcgcaccaggtacttctgaaagcgcaaccccggaatccggcccaggtacctctgaaagcgcaaccccggagtccggcccaggtagccctgctggctctccaacctccaccgaagaaggtacctctgaaagcgcaacccctgaatccggcccaggtagcgaaccggcaacctccggttctgaaaccccaggtacctctgaaagcgctactccggagtctggcccaggtacctctactgaaccgtctgagggtagcgctccaggtacttctactgaaccgtccgaaggtagcgcaccaggtacttctaccgaaccgtccgaaggcagcgctccaggtacctctactgaaccttccgagggcagcgctccaggtacctctaccgaaccttctgaaggtagcgcaccaggtacttctaccgaaccgtccgagggtagcgcaccaggtagcccagcaggttctcctacctccaccgaggaaggtacttctaccgaaccgtccgagggtagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtacttctgaaagcgctactcctgagtccggcccaggtagcccggctggctctccgacttccaccgaggaaggtagcccggctggctctccaacttctactgaagaaggtagcccggcaggctctccgacctctactgaggaaggtacttctgaaagcgcaaccccggagtccggcccaggtacctctaccgaaccgtctgagggcagcgcaccaggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtagccctgctggctctccaacctccaccgaagaaggtacctctgaaagcgcaacccctgaatccggcccaggtagcgaaccggcaacctccggttctgaaaccccaggtacttctgaaagcgctactcctgagtccggcccaggtagcccggctggctctccgacttccaccgaggaaggtagcccggctggctctccaacttctactgaagaaggtacttctaccgaaccttccgagggcagcgcaccaggtacttctgaaagcgctacccctgagtccggcccaggtacttctgaaagcgctactcctgaatccggtccaggtacttctgaaagcgctaccccggaatctggcccaggtagcgaaccggctacttctggttctgaaaccccaggtagcgaaccggctacctccggttctgaaactccaggtagcccagcaggctctccgacttccactgaggaaggtacttctactgaaccttccgaaggcagcgcaccaggtacctctactgaaccttctgagggcagcgctccaggtagcgaacctgcaacctctggctctgaaaccccaggtacctctgaaagcgctactcctgaatctggcccaggtacttctactgaaccgtccgagggcagcgcaccaggttttccgactattccgctgtctcgtctgtttgataatgctatgctgcgtgcgcaccgtctgcaccagctggcctttgatacttaccaggaatttgaagaagcctacattcctaaagagcagaagtactctttcctgcaaaacccacagacttctctctgcttcagcgaatctattccgacgccttccaatcgcgaggaaactcagcaaaagtccaatctggaactactccgcatttctctgcttctgattcagagctggctagaaccagtgcaatttctgcgttccgtcttcgccaatagcctagtttatggcgcatccgacagcaacgtatacgatctcctgaaagatctcgaggaaggcattcagaccctgatgggtcgtctcgaggatggctctccgcgtactggtcagatcttcaagcagacttactctaaatttgatactaacagccacaatgacgatgcgcttctaaaaaactatggtctgctgtattgttttcgtaaagatatggacaaagttgaaaccttcctgcgtattgttcagtgtcgttccgttgagggcagctgtggtttctaaggtggtacctctgaaagcgcaactcctgagtctggcccaggtagcgaacctgctacctccggctctgagactccaggtacctctgaaagcgcaaccccggaatctggtccaggtagcgaacctgcaacctctggctctgaaaccccaggtacctctgaaagcgctactcctgaatctggcccaggtacttctactgaaccgtccgagggcagcgcaccaggtagccctgctggctctccaacctccaccgaagaaggtacctctgaaagcgcaacccctgaatccggcccaggtagcgaaccggcaacctccggttctgaaaccccaggtacttctgaaagcgctactcctgagtccggcccaggtagcccggctggctctccgacttccaccgaggaaggtagcccggctggctctccaacttctactgaagaaggtacttctaccgaaccttccgagggcagcgcaccaggtacttctgaaagcgctacccctgagtccggcccaggtacttctgaaagcgctactcctgaatccggtccaggtacttctgaaagcgctaccccggaatctggcccaggtagcgaaccggctacttctggttctgaaaccccaggtagcgaaccggctacctccggttctgaaactccaggtagcccagcaggctctccgacttccactgaggaaggtacttctactgaaccttccgaaggcagcgcaccaggtacctctactgaaccttctgagggcagcgctccaggtagcgaacctgcaacctctggctctgaaaccccaggtacctctgaaagcgctactcctgaatctggcccaggtacttctactgaaccgtccgagggcagcgcacca

SEQ ID NO: 12 - DNA, 3204 bp, Artificial Sequence
  Description of Artificial Sequence Synthetic polynucleotide
ggtacttctactgaaccgtctgaaggcagcgcaccaggtagcgaaccggctacttccggttctgaaaccccaggtagcccagcaggttctccaacttctactgaagaaggttctaccagctctaccgcagaatctcctggtccaggtacctctactccggaaagcggctctgcatctccaggttctactagcgaatctccttctggcactgcaccaggttctactagcgaatccccgtctggtactgctccaggtacttctactcctgaaagcggttccgcttctccaggtacctctactccggaaagcggttctgcatctccaggtagcgaaccggcaacctccggctctgaaaccccaggtacctctgaaagcgctactcctgaatccggcccaggtagcccggcaggttctccgacttccactgaggaaggtacctctactgaaccttctgagggcagcgctccaggtacttctgaaagcgctaccccggagtccggtccaggtacttctactgaaccgtccgaaggtagcgcaccaggtacttctaccgaaccgtccgagggtagcgcaccaggtagcccagcaggttctcctacctccaccgaggaaggtacttctaccgaaccgtccgagggtagcgcaccaggtacttctaccgaaccttccgagggcagcgcaccaggtacttctgaaagcgctacccctgagtccggcccaggtacttctgaaagcgctactcctgaatccggtccaggtacctctactgaaccttccgaaggcagcgctccaggtacctctaccgaaccgtccgagggcagcgcaccaggtacttctgaaagcgcaacccctgaatccggtccaggtacttctactgaaccttccgaaggtagcgctccaggtagcgaacctgctacttctggttctgaaaccccaggtagcccggctggctctccgacctccaccgaggaaggtagctctaccccgtctggtgctactggttctccaggtactccgggcagcggtactgcttcttcctctccaggtagctctaccccttctggtgctactggctctccaggtacctctaccgaaccgtccgagggtagcgcaccaggtacctctactgaaccgtctgagggtagcgctccaggtagcgaaccggcaacctccggttctgaaactccaggtagccctgctggctctccgacttctactgaggaaggtagcccggctggttctccgacttctactgaggaaggtacttctaccgaaccttccgaaggtagcgctccaggtgcaagcgcaagcggcgcgccaagcacgggaggtacttctgaaagcgctactcctgagtccggcccaggtagcccggctggctctccgacttccaccgaggaaggtagcccggctggctctccaacttctactgaagaaggttctaccagctctaccgctgaatctcctggcccaggttctactagcgaatctccgtctggcaccgcaccaggtacttcccctagcggtgaatcttctactgcaccaggtacccctggcagcggtaccgcttcttcctctccaggtagctctaccccgtctggtgctactggctctccaggttctagcccgtctgcatctaccggtaccggcccaggtagcgaaccggcaacctccggctctgaaactccaggtacttctgaaagcgctactccggaatccggcccaggtagcgaaccggctacttccggctctgaaaccccaggttccaccagctctactgcagaatctccgggcccaggttctactagctctactgcagaatctccgggtccaggtacttctcctagcggcgaatcttctaccgctccaggtagcgaaccggcaacctctggctctgaaactccaggtagcgaacctgcaacctccggctctgaaaccccaggtacttctactgaaccttctgagggcagcgcaccaggttctaccagctctaccgcagaatctcctggtccaggtacctctactccggaaagcggctctgcatctccaggttctactagcgaatctccttctggcactgcaccaggtacttctaccgaaccgtccgaaggcagcgctccaggtacctctactgaaccttccgagggcagcgctccaggtacctctaccgaaccttctgaaggtagcgcaccaggtagctctactccgtctggtgcaaccggctccccaggttctagcccgtctgcttccactggtactggcccaggtgcttccccgggcaccagctctactggttctccaggtagcgaacctgctacctccggttctgaaaccccaggtacctctgaaagcgcaactccggagtctggtccaggtagccctgcaggttctcctacctccactgaggaaggtagctctactccgtctggtgcaaccggctccccaggttctagcccgtctgcttccactggtactggcccaggtgcttccccgggcaccagctctactggttctccaggtacctctgaaagcgctactccggagtctggcccaggtacctctactgaaccgtctgagggtagcgctccaggtacttctactgaaccgtccgaaggtagcgcaccaggttttccgactattccgctgtctcgtctgtttgataatgctatgctgcgtgcgcaccgtctgcaccagctggcctttgatacttaccaggaatttgaagaagcctacattcctaaagagcagaagtactctttcctgcaaaacccacagacttctctctgcttcagcgaatctattccgacgccttccaatcgcgaggaaactcagcaaaagtccaatctggaactactccgcatttctctgcttctgattcagagctggctagaaccagtgcaatttctgcgttccgtcttcgccaatagcctagtttatggcgcatccgacagcaacgtatacgatctcctgaaagatctcgaggaaggcattcagaccctgatgggtcgtctcgaggatggctctccgcgtactggtcagatcttcaagcagacttactctaaatttgatactaacagccacaatgacgatgcgcttctaaaaaactatggtctgctgtattgttttcgtaaagatatggacaaagttgaaaccttcctgcgtattgttcagtgtcgttccgttgagggcagctgtggtttctaa

SEQ ID NO: 13 - protein, 48 aa, Artificial Sequence
  Description of Artificial Sequence Synthetic polypeptide
MAEPAGSPTSTEEGTPGSGTASSSPGSSTPSGATGSPGASPGTSSTGS

SEQ ID NO: 14 - protein, 48 aa, Artificial Sequence
  Description of Artificial Sequence Synthetic polypeptide
MAEPAGSPTSTEEGASPGTSSTGSPGSSTPSGATGSPGSSTPSGATGS

SEQ ID NO: 15 - protein, 144 aa, Artificial Sequence
  Description of Artificial Sequence Synthetic polypeptide
GSEPATSGSETPGTSESATPESGPGSEPATSGSETPGSPAGSPTSTEEGTSTEPSEGSAPGSEPATSGSETPGSEPATSGSETPGSEPATSGSETPGTSTEPSEGSAPGTSESATPESGPGSEPATSGSETPGTSTEPSEGSAP

SEQ ID NO: 16 - protein, 144 aa, Artificial Sequence
  Description of Artificial Sequence Synthetic polypeptide
GTSTPESGSASPGTSPSGESSTAPGTSPSGESSTAPGSTSSTAESPGPGSTSESPSGTAPGSTSSTAESPGPGTSPSGESSTAPGTSTPESGSASPGSTSSTAESPGPGTSPSGESSTAPGTSPSGESSTAPGTSPSGESSTAP

SEQ ID NO: 17 - protein, 288 aa, Artificial Sequence
  Description of Artificial Sequence Synthetic polypeptide
GTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 18 - protein, 504 aa, Artificial Sequence
  Description of Artificial Sequence Synthetic polypeptide
GASPGTSSTGSPGSSPSASTGTGPGSSPSASTGTGPGTPGSGTASSSPGSSTPSGATGSPGSXPSASTGTGPGASPGTSSTGSPGTPGSGTASSSPGSSTPSGATGSPGTPGSGTASSSPGASPGTSSTGSPGASPGTSSTGSPGTPGSGTASSSPGSSTPSGATGSPGASPGTSSTGSPGTPGSGTASSSPGSSTPSGATGSPGSXPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSP

SEQ ID NO: 19 - protein, 540 aa, Artificial Sequence
  Description of Artificial Sequence Synthetic polypeptide
GSTSSTAESPGPGSTSSTAESPGPGSTSESPSGTAPGSTSSTAESPGPGSTSSTAESPGPGTSTPESGSASPGSTSESPSGTAPGTSPSGESSTAPGSTSESPSGTAPGSTSESPSGTAPGTSPSGESSTAPGSTSESPSGTAPGSTSESPSGTAPGTSPSGESSTAPGSTSESPSGTAPGSTSESPSGTAPGSTSESPSGTAPGTSTPESGSASPGSTSESPSGTAPGTSTPESGSASPGSTSSTAESPGPGSTSSTAESPGPGTSTPESGSASPGTSTPESGSASPGSTSESPSGTAPGTSTPESGSASPGTSTPESGSASPGSTSESPSGTAPGSTSESPSGTAPGSTSESPSGTAPGSTSSTAESPGPGTSTPESGSASPGTSTPESGSASPGSTSESPSGTAPGSTSESPSGTAPGTSTPESGSASPGSTSESPSGTAPGSTSESPSGTAPGTSTPESGSASPGTSPSGESSTAPGSTSSTAESPGPGTSPSGESSTAPGSTSSTAESPGPGTSTPESGSASPGSTSESPSGTAP

SEQ ID NO: 20 - protein, 576 aa, Artificial Sequence
  Description of Artificial Sequence Synthetic polypeptide
GSSESGSSEGGPGSGGEPSESGSSGSSESGSSEGGPGSSESGSSEGGPGSSESGSSEGGPGSSESGSSEGGPGSSESGSSEGGPGESPGGSSGSESGSEGSSGPGESSGSSESGSSEGGPGSSESGSSEGGPGSSESGSSEGGPGSGGEPSESGSSGESPGGSSGSESGESPGGSSGSESGSGGEPSESGSSGSSESGSSEGGPGSGGEPSESGSSGSGGEPSESGSSGSEGSSGPGESSGESPGGSSGSESGSGGEPSESGSSGSGGEPSESGSSGSGGEPSESGSSGSSESGSSEGGPGESPGGSSGSESGESPGGSSGSESGESPGGSSGSESGESPGGSSGSESGESPGGSSGSESGSSESGSSEGGPGSGGEPSESGSSGSEGSSGPGESSGSSESGSSEGGPGSGGEPSESGSSGSSESGSSEGGPGSGGEPSESGSSGESPGGSSGSESGESPGGSSGSESGSSESGSSEGGPGSGGEPSESGSSGSSESGSSEGGPGSGGEPSESGSSGSGGEPSESGSSGESPGGSSGSESGSEGSSGPGESSGSSESGSSEGGPGSEGSSGPGESS

SEQ ID NO: 21 - protein, 576 aa, Artificial Sequence
  Description of Artificial Sequence Synthetic polypeptide
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAP

SEQ ID NO: 22 - protein, 576 aa, Artificial Sequence
  Description of Artificial Sequence Synthetic polypeptide
GSTSSTAESPGPGSTSSTAESPGPGSTSESPSGTAPGSTSSTAESPGPGSTSSTAESPGPGTSTPESGSASPGSTSESPSGTAPGTSPSGESSTAPGSTSESPSGTAPGSTSESPSGTAPGTSPSGESSTAPGSTSESPSGTAPGSTSESPSGTAPGTSPSGESSTAPGSTSESPSGTAPGSTSESPSGTAPGSTSESPSGTAPGTSTPESGSASPGSTSESPSGTAPGTSTPESGSASPGSTSSTAESPGPGSTSSTAESPGPGTSTPESGSASPGTSTPESGSASPGSTSESPSGTAPGTSTPESGSASPGTSTPESGSASPGSTSESPSGTAPGSTSESPSGTAPGSTSESPSGTAPGSTSSTAESPGPGTSTPESGSASPGTSTPESGSASPGSTSESPSGTAPGSTSESPSGTAPGTSTPESGSASPGSTSESPSGTAPGSTSESPSGTAPGTSTPESGSASPGTSPSGESSTAPGSTSSTAESPGPGTSPSGESSTAPGSTSSTAESPGPGTSTPESGSASPGSTSESPSGTAPGSTSSTAESPGPGTSTPESGSASPGTSTPESGSASP

SEQ ID NO: 23 - protein, 625 aa, Artificial Sequence
  Description of Artificial Sequence Synthetic polypeptide
MAEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAP

SEQ ID NO: 24 - protein, 836 aa, Artificial Sequence
  Description of Artificial Sequence Synthetic polypeptide
GSSESGSSEGGPGSSESGSSEGGPGESPGGSSGSESGSGGEPSESGSSGESPGGSSGSESGESPGGSSGSESGSSESGSSEGGPGSSESGSSEGGPGSSESGSSEGGPGESPGGSSGSESGESPGGSSGSESGESPGGSSGSESGSSESGSSEGGPGSSESGSSEGGPGSSESGSSEGGPGSSESGSSEGGPGSSESGSSEGGPGSSESGSSEGGPGSGGEPSESGSSGESPGGSSGSESGESPGGSSGSESGSGGEPSESGSSGSEGSSGPGESSGSSESGSSEGGPGSGGEPSESGSSGSEGSSGPGESSGSSESGSSEGGPGSGGEPSESGSSGESPGGSSGSESGSGGEPSESGSSGSGGEPSESGSSGSSESGSSEGGPGSGGEPSESGSSGSGGEPSESGSSGSEGSSGPGESSGESPGGSSGSESGSEGSSGPGESSGSEGSSGPGESSGSGGEPSESGSSGSSESGSSEGGPGSSESGSSEGGPGESPGGSSGSESGSGGEPSESGSSGSEGSSGPGESSGESPGGSSGSESGSEGSSGPGSSESGSSEGGPGSGGEPSESGSSGSEGSSGPGESSGSEGSSGPGESSGSEGSSGPGESSGSGGEPSESGSSGSGGEPSESGSSGESPGGSSGSESGESPGGSSGSESGSGGEPSESGSSGSEGSSGPGESSGESPGGSSGSESGSSESGSSEGGPGSSESGSSEGGPGSSESGSSEGGPGSGGEPSESGSSGSSESGSSEGGPGESPGGSSGSESGSGGEPSESGSSGSSESGSSEGGPGESPGGSSGSESGSGGEPSESGSSGESPGGSSGSESGSGGEPSESGSS

SEQ ID NO: 25 - protein, 864 aa, Artificial Sequence
  Description of Artificial Sequence Synthetic polypeptide
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 26 - protein, 875 aa, Artificial Sequence
  Description of Artificial Sequence Synthetic polypeptide
GSTSESPSGTAPGTSPSGESSTAPGSTSESPSGTAPGSTSESPSGTAPGTSTPESGSASPGTSTPESGSASPGSTSESPSGTAPGSTSESPSGTAPGTSPSGESSTAPGSTSESPSGTAPGTSPSGESSTAPGTSPSGESSTAPGSTSSTAESPGPGTSPSGESSTAPGTSPSGESSTAPGSTSSTAESPGPGTSTPESGSASPGTSTPESGSASPGSTSESPSGTAPGSTSESPSGTAPGTSTPESGSASPGSTSSTAESPGPGTSTPESGSASPGSTSESPSGTAPGTSPSGESSTAPGSTSSTAESPGPGTSPSGESSTAPGTSTPESGSASPGSTSSTAESPGPGSTSSTAESPGPGSTSSTAESPGPGSTSSTAESPGPGTSPSGESSTAPGSTSESPSGTAPGSTSESPSGTAPGTSTPESGPXXXGASASGAPSTXXXXSESPSGTAPGSTSESPSGTAPGSTSESPSGTAPGSTSESPSGTAPGSTSESPSGTAPGSTSESPSGTAPGTSTPESGSASPGTSPSGESSTAPGTSPSGESSTAPGSTSSTAESPGPGTSPSGESSTAPGTSTPESGSASPGSTSESPSGTAPGSTSESPSGTAPGTSPSGESSTAPGSTSESPSGTAPGTSTPESGSASPGTSTPESGSASPGSTSESPSGTAPGTSTPESGSASPGSTSSTAESPGPGSTSESPSGTAPGSTSESPSGTAPGTSPSGESSTAPGSTSSTAESPGPGTSPSGESSTAPGTSTPESGSASPGTSPSGESSTAPGTSPSGESSTAPGTSPSGESSTAPGSTSSTAESPGPGSTSSTAESPGPGTSPSGESSTAPGSSPSASTGTGPGSSTPSGATGSPGSSTPSGATGSP

SEQ ID NO: 27 - protein, 864 aa, Artificial Sequence
  Description of Artificial Sequence Synthetic polypeptide
GASPGTSSTGSPGSSPSASTGTGPGSSPSASTGTGPGTPGSGTASSSPGSSTPSGATGSPGSSPSASTGTGPGASPGTSSTGSPGTPGSGTASSSPGSSTPSGATGSPGTPGSGTASSSPGASPGTSSTGSPGASPGTSSTGSPGTPGSGTASSSPGSSTPSGATGSPGASPGTSSTGSPGTPGSGTASSSPGSSTPSGATGSPGSSPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSPGASPGTSSTGSPGTPGSGTASSSPGASPGTSSTGSPGASPGTSSTGSPGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSPGSSTPSGATGSPGASPGTSSTGSP

SEQ ID NO: 28 - protein, 875 aa, Artificial Sequence
  Description of Artificial Sequence Synthetic polypeptide
GTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGSPAGSPTSTEEGSSTPSGATGSPGSSPSASTGTGPGASPGTSSTGSPGTSESATPESGPGTSTEPSEGSAPGTSTEPSEGSAP

SEQ ID NO: 29 - protein, 913 aa, Artificial Sequence
  Description of Artificial Sequence Synthetic polypeptide
MAEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 30 - protein, 924 aa, Artificial Sequence
  Description of Artificial Sequence Synthetic polypeptide
MAEPAGSPTSTEEGASPGTSSTGSPGSSTPSGATGSPGSSTPSGATGSPGTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGASASGAPSTGGTSESATPESGPGSPAGSPTSTEEGSPAGSPTSTEEGSTSSTAESPGPGSTSESPSGTAPGTSPSGESSTAPGTPGSGTASSSPGSSTPSGATGSPGSSPSASTGTGPGSEPATSGSETPGTSESATPESGPGSEPATSGSETPGSTSSTAESPGPGSTSSTAESPGPGTSPSGESSTAPGSEPATSGSETPGSEPATSGSETPGTSTEPSEGSAPGSTSSTAESPGPGTSTPESGSASPGSTSESPSGTAPGTSTEPSEGSAPGTSTEPSEGSAPGTSTEPSEGSAPGSSTPSGATGSPGSSPSASTGTGPGASPGTSSTGSPGSEPATSGSETPGTSESATPESGPGSPAGSPTSTEEGSSTPSGATGSPGSSPSASTGTGPGASPGTSSTGSPGTSESATPESGPGTSTEPSEGSAPGTSTEPSEGSAP

SEQ ID NO: 31 - protein, 1318 aa, Artificial Sequence
  Description of Artificial Sequence Synthetic polypeptide
GTSTEPSEGSAPGSEPATSGSETPGSPAGSPTSTEEGSTSSTAESPGPGTSTPESGSASPGSTSESPSGTAPGSTSESPSGTAPGTSTPESGSASPGTSTPESGSASPGSEPATSGSETPGTSESATPESGPGSPAGSPTSTEEGTSTEPSEGSAPGTSESATPESGPGTSTEPSEGSAPGTSTEPSEGSAPGSPAGSPTSTEEGTSTEPSEGSAPGTSTEPSEGSAPGTSESATPESGPGTSESATPESGPGTSTEPSEGSAPGTSTEPSEGSAPGTSESATPESGPGTSTEPSEGSAPGSEPATSGSETPGSPAGSPTSTEEGSSTPSGATGSPGTPGSGTASSSPGSSTPSGATGSPGTSTEPSEGSAPGTSTEPSEGSAPGSEPATSGSETPGSPAGSPTSTEEGSPAGSPTSTEEGTSTEPSEGSAPGPEPTGPAPSGGSEPATSGSETPGTSESATPESGPGSPAGSPTSTEEGTSESATPESGPGSPAGSPTSTEEGSPAGSPTSTEEGTSESATPESGPGSPAGSPTSTEEGSPAGSPTSTEEGSTSSTAESPGPGSTSESPSGTAPGTSPSGESSTAPGSTSESPSGTAPGSTSESPSGTAPGTSPSGESSTAPGTSTEPSEGSAPGTSESATPESGPGTSESATPESGPGSEPATSGSETPGTSESATPESGPGTSESATPESGPGTSTEPSEGSAPGTSESATPESGPGTSTEPSEGSAPGTSPSGESSTAPGTSPSGESSTAPGTSPSGESSTAPGTSTEPSEGSAPGSPAGSPTSTEEGTSTEPSEGSAPGSSPSASTGTGPGSSTPSGATGSPGSSTPSGATGSPGSSTPSGATGSPGSSTPSGATGSPGASPGTSSTGSPGASASGAPSTGGTSPSGESSTAPGSTSSTAESPGPGTSPSGESSTAPGTSESATPESGPGTSTEPSEGSAPGTSTEPSEGSAPGSSPSASTGTGPGSSTPSGATGSPGASPGTSSTGSPGTSTPESGSASPGTSPSGESSTAPGTSPSGESSTAPGTSESATPESGPGSEPATSGSETPGTSTEPSEGSAPGSTSESPSGTAPGSTSESPSGTAPGTSTPESGSASPGSPAGSPTSTEEGTSESATPESGPGTSTEPSEGSAPGSPAGSPTSTEEGTSESATPESGPGSEPATSGSETPGSSTPSGATGSPGASPGTSSTGSPGSSTPSGATGSPGSTSESPSGTAPGTSPSGESSTAPGSTSSTAESPGPGSSTPSGATGSPGASPGTSSTGSPGTPGSGTASSSPGSPAGSPTSTEEGSPAGSPTSTEEGTSTEPSEGSAP

SEQ ID NO: 32 - protein, 864 aa, Artificial Sequence
  Description of Artificial Sequence Synthetic polypeptide
GTSTEPSEPGSAGTSTEPSEPGSAGSEPATSGTEPSGSGASEPTSTEPGSEPATSGTEPSGSEPATSGTEPSGSEPATSGTEPSGSGASEPTSTEPGTSTEPSEPGSAGSEPATSGTEPSGTSTEPSEPGSAGSEPATSGTEPSGSEPATSGTEPSGTSTEPSEPGSAGTSTEPSEPGSAGSEPATSGTEPSGSEPATSGTEPSGTSEPSTSEPGAGSGASEPTSTEPGTSEPSTSEPGAGSEPATSGTEPSGSEPATSGTEPSGTSTEPSEPGSAGTSTEPSEPGSAGSGASEPTSTEPGSEPATSGTEPSGSEPATSGTEPSGSEPATSGTEPSGSEPATSGTEPSGTSTEPSEPGSAGSEPATSGTEPSGSGASEPTSTEPGTSTEPSEPGSAGSEPATSGTEPSGSGASEPTSTEPGTSTEPSEPGSAGSGASEPTSTEPGSEPATSGTEPSGSGASEPTSTEPGSEPATSGTEPSGSGASEPTSTEPGTSTEPSEPGSAGSEPATSGTEPSGSGASEPTSTEPGTSTEPSEPGSAGSEPATSGTEPSGTSTEPSEPGSAGSEPATSGTEPSGTSTEPSEPGSAGTSTEPSEPGSAGTSTEPSEPGSAGTSTEPSEPGSAGTSTEPSEPGSAGTSTEPSEPGSAGTSEPSTSEPGAGSGASEPTSTEPGTSTEPSEPGSAGTSTEPSEPGSAGTSTEPSEPGSAGSEPATSGTEPSGSGASEPTSTEPGSEPATSGTEPSGSEPATSGTEPSGSEPATSGTEPSGSEPATSGTEPSGTSEPSTSEPGAGSEPATSGTEPSGSGASEPTSTEPGTSTEPSEPGSAGSEPATSGTEPSGSGASEPTSTEPGTSTEPSEPGSA

SEQ ID NO: 33 - protein, 864 aa, Artificial Sequence
  Description of Artificial Sequence Synthetic polypeptide
GSETATSGSETAGTSESATSESGAGSTAGSETSTEAGTSESATSESGAGSETATSGSETAGSETATSGSETAGTSTEASEGSASGTSTEASEGSASGTSESATSESGAGSETATSGSETAGTSTEASEGSASGSTAGSETSTEAGTSESATSESGAGTSESATSESGAGSETATSGSETAGTSESATSESGAGTSTEASEGSASGSETATSGSETAGSETATSGSETAGTSTEASEGSASGSTAGSETSTEAGTSESATSESGAGTSTEASEGSASGSETATSGSETAGSTAGSETSTEAGSTAGSETSTEAGSETATSGSETAGTSESATSESGAGTSESATSESGAGSETATSGSETAGTSESATSESGAGTSESATSESGAGSETATSGSETAGSETATSGSETAGTSTEASEGSASGSTAGSETSTEAGSETATSGSETAGTSESATSESGAGSTAGSETSTEAGSTAGSETSTEAGSTAGSETSTEAGTSTEASEGSASGSTAGSETSTEAGSTAGSETSTEAGTSTEASEGSASGSTAGSETSTEAGSETATSGSETAGTSTEASEGSASGTSESATSESGAGSETATSGSETAGTSESATSESGAGTSESATSESGAGSETATSGSETAGTSESATSESGAGSETATSGSETAGTSTEASEGSASGTSTEASEGSASGSTAGSETSTEAGSTAGSETSTEAGSETATSGSETAGTSESATSESGAGTSESATSESGAGSETATSGSETAGSETATSGSETAGSETATSGSETAGTSTEASEGSASGTSESATSESGAGSETATSGSETAGSETATSGSETAGTSESATSESGAGTSESATSESGAGSETATSGSETA

SEQ ID NO: 34 - protein, 912 aa, Artificial Sequence
  Description of Artificial Sequence Synthetic polypeptide
AEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 35 - protein, 146 aa, Artificial Sequence
  Description of Artificial Sequence Synthetic polypeptide
GGTSESATPESGPGTSTEPSEGSAPGTSTEPSEGSAPGTSESATPESGPGTSTEPSEGSAPGTSTEPSEGSAPGTSESATPESGPGTSTEPSEGSAPGTSTEPSEGSAPGTSTEPSEGSAPGSPAGSPTSTEEGTSTEPSEGSAPG

SEQ ID NO: 36 - protein, 47 aa, Artificial Sequence
  Description of Artificial Sequence Synthetic polypeptide
AEPAGSPTSTEEGTPGSGTASSSPGSSTPSGATGSPGASPGTSSTGS

SEQ ID NO: 37 - protein, 47 aa, Artificial Sequence
  Description of Artificial Sequence Synthetic polypeptide
AEPAGSPTSTEEGASPGTSSTGSPGSSTPSGATGSPGSSTPSGATGS

SEQ ID NO: 38 - protein, 912 aa, Artificial Sequence; identical to SEQ ID NO: 34
  Description of Artificial Sequence Synthetic polypeptide

SEQ ID NO: 39 - protein, 913 aa, Artificial Sequence
  Description of Artificial Sequence Synthetic polypeptide
AEPAGSPTSTEEGTPGSGTASSSPGSSTPSGATGSPGASPGTSSTGSPGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPG

SEQ ID NO: 40 - protein, 144 aa, Artificial Sequence
  Description of Artificial Sequence Synthetic polypeptide
TSESATPESGPGTSTEPSEGSAPGTSTEPSEGSAPGTSESATPESGPGTSTEPSEGSAPGTSTEPSEGSAPGTSESATPESGPGTSTEPSEGSAPGTSTEPSEGSAPGTSTEPSEGSAPGSPAGSPTSTEEGTSTEPSEGSAPG

SEQ ID NO: 41 - protein, 191 aa, Homo sapiens [sequence omitted]